SEQUENCE LISTING (16 sequences)

SEQ ID NO: 1 - DNA, 11850 bp, synthetic construct
  source 1..11850: mol_type = other DNA
gatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactcgaagtcacagccaaaagccagacagagtctgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaagctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggta

SEQ ID NO: 2 - DNA, 10058 bp, synthetic construct
  source 1..10058: mol_type = other DNA
agcttgcatgcctgcaggtcgactctagaggatccccgggtacgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaaagctttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctaccactttgtacaagaaagctgggtttaagccttcttgatagcagcagcgagttcatcgtaagcaacttcccaagccctagacaattcatctgaccatttatcaccaacagcagctttaatagttttaagcaaagcttctttcacaacaacgaactgaggatctgtaacagccttttgagcatgaactgaacccaaagcagcatcagcaacaactgtcccagaagctttaagctgaccagctgaatctctcaccagagcaaaaagcttttctgcatgtccagtcaacttaggatttgttggatcaacaccgttagccaaaaatgaaaacaaatccttagcagcaggagctttttccaaaattgatgtgtagaaaaccacagaatattgtggaatatttgccttgaaagcttcaaatgatgatgaaacaagagcatcttgcttttcagtaaaagcaaccatggtggcagcctgcttttttgtacaaacttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggccaacttttctatacaaagttg

SEQ ID NO: 3 - DNA, 15312 bp, synthetic construct
  source 1..15312: mol_type = other DNA
agcttgcatgcctgcaggtcgactctagaggatcgacttcaaccttattagtgaatggacaataaaggttataagctcctttactgtgaaagcccaccagtaacatcaccttgcttatatcattcagcttctttctagtaacatttggaacgtgtttataacagaaaaaaacccaaaaactctgaaaagactcacacttttcttatctccagtccacctctcaaaaggaacaatttccttcagcttcttggttggacacctgttgagcacatatgctgcagtggcaacagtttctccccacaaagtgttaggaagcttcttctccttcagcatgttccttgtcatatcaagcaaagttcggttttcaacaagaccattatgttgaggagtatatggatcagtcacttcatgctcaattccattctctttacagaacttcttgaactctgtagagttatactcacctccaccatcagttctgagaatcttcagaagtctgaccactttatttctcagccttgattatgaatttcttaaattcagcaaacacctcgtgtttgaattttataagggatacccatgtcatccttgtgaactcatccataaatgacataaagtattattccctcctagtgaaaggtttgtaatgggccacacacataagaatgcactactcctaaagcatgttttgctctttgagctacttttgatgaaaatggcagtcttggttgcttccctttcatgcacacattacatgacttttttggtttcttaattgtaggaattccacgtaccagtttctttgaattcaaattccctaagctcctaaagttcaaatgaccaaatcttttgttccacaactcactttccttcacaacacttgttgcgctaaggcattcagagtctgcagttttaacattcgccttgaatgttttactccttccatgttctgactccataatcaacttctgataacagtcatacagcttcaaaagaatgtcattcatggtaactggaaatcccttttcaattaattgacctacactcatcagattgctcttcatgccaagaacgtaccaagacgttctgaattaatgcagattttctattattcataatcactctaacattccccattcctttagcatttagttacttatcatcagcacatctaatcttggttttcttcctagagtcaaaatcaaccagccatttcttatttccagtatgatggtttgaacaaccagtgtccatatatcaccagtcttctatagacgcactatcataactagaagccattaatagcacatgttcatcatggtgctcagatccttagaatgttcaattgctacaacgatgtaatcaaactgatgagtaagagatctaagtaccttctcaatgatactttcctcataaagagtttctccatgcgacttcatctcatttgtgatcagaatcactctagagatgtagtcagataacttctcattgttcttcatgcttagattctcatactgctcacgtagagactgaagtttcaccttctacactgatgcatcactatcgtagcaccacaccagtctgtctcacacaaccttttccgtcattgaatcaacgattttcttaaacacgttcacatccacacactgatggatgtagaacaacgcattctgatccttcttcctcatatcacactgagcatttctttgcgcatccgttgcattttctagaagtgaagcataaacttcgttgatgagatcaagaacatcttgagcaccaaataacacacacatctgaatcatccaacgattccagttgttgtcgtcgaacaatggnagcntggtgcacagattcacaacgatatattataanttttgttttatgaaatttaagaacaaatttccattattcttaaaatgtttacacactgatgtagactgcaaaaggaataaagatacaatttgttcacaccactcacttgcgtaaatataagtgagagttaatgagaaatactaaaataccctctaaaattatgaattaattctaacaatctctaatgttagtataatccattaaacactttgatggcaggtataacaagggtgtaagttagtgtatacatattaggctcttattatttttatattatctctgcttttcttcttcatgttctcactaatatgatattatctcccttccctaaattatttatatttattagaaaaagagtttcattttttaaaaatatattaccgtaatttttcaaaaaataaaatttaaatatattttataaaaaaattatttaataatttatttacattaatgcataaatataaataaatactgtcatttaatatttaaccttttaacaataaattatatttatttaattcaactaatataagctaagttatctcatccaaccaattaaaaagatcatttgaaaatacctttttatttagtttgtggcggtttcaactgtcaaaaaaaaggaatttttacgacgatataaatttaaaccagcaaaaaattgaagcagttaagcgaaccaactcatggtatgtggatatatttatctttgtcgtttatatcggattcgaatctctataatgatgaaaaattaatatcaaactttaaataagaacgtcatttatagagccattttgggaaacacatatttcatgtacacgtgattcgcaaatttccaataactctatatatagccctcctcagtttcatgcatttgctcacaacataaccttccttgaatctggaaaacgtcacattgcttccgcatatcgggtcagcaacggctaaaatccgcttgaatatgttcacacaagccgctcaaaacatgattgacgccgtatacggaagaacgccgaaaaaccttactaaggaatttcaataagaagaaaaatcccggttggttcagccggggtttatttttcgctagataaaaagtactatttttaaattctttctattcctttctttcgttgctgatacaatgaaaaggaatcagcttcacatgatgaaaatgggaggtattgctttgaaaaaacgattatcgtggatttccgtttgtttactggtgcttgtctccgcggcggggatgctgttttcaacagctgccaaaacggaaacatcttctcacaaggcacacacagaagcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataacgaaaaaaacggcttccctgcggaggccgtttttttcagctttacataaagtgtgtaataaatttttcttcaaactctgatcggtcaatttcactttgctagccaccaccaccaccaccacgtgtgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccgggtacgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaaagctttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctaccactttgtacaagaaagctgggtttaagccttcttgatagcagcagcgagttcatcgtaagcaacttcccaagccctagacaattcatctgaccatttatcaccaacagcagctttaatagttttaagcaaagcttctttcacaacaacgaactgaggatctgtaacagccttttgagcatgaactgaacccaaagcagcatcagcaacaactgtcccagaagctttaagctgaccagctgaatctctcaccagagcaaaaagcttttctgcatgtccagtcaacttaggatttgttggatcaacaccgttagccaaaaatgaaaacaaatccttagcagcaggagctttttccaaaattgatgtgtagaaaaccacagaatattgtggaatatttgccttgaaagcttcaaatgatgatgaaacaagagcatcttgcttttcagtaaaagcaaccatggtggcggatcccttcttttgcactgtgaatgattagaataatttctaaaaatctcaatatgaggatgccatatttataatagaataaaataaaatgtgaacaaagaaagagataaagtagttcactttttgaaatctaagagagaaatgggaacaagaaagagacaaaagtagtttcaaacaaacttctcttctaagtttagtccctttttaaaatatgaaacccaatacgtctgattaagaatagaaaaatatcaaattttcaatataatttatactaatcgttttgaatttttcatactgatatagtgtacgtttcatcataacaaccaaaacgttgttgtttcacaacaataatatagtagtagttaatttattatttagtaataagtggtcctaaaaattagataaatattactatgataatataaaaatatttgagtcagtcctaaaaaattatttagtattcatacatgaatcaaactaattagttaagtgtcaacaattggacaagtggcatggaggttgtaaaagaatgacataagccaactgctatttttatccaaaaaaaagaagacaacttgacaactacatttcttttatttttataaatttactaatatcttctatgcaaaattattcggtgcctttctaaactttaaggtttttatttgatgtacacctaaattatattttattttaatcacttcactgaacttgtttattccttcatcatatacacctactcctattatgactacaagttggcaaaagtaatgatatgaatttctacttaaataaataatagtcacctagataaattaatttaacaaaagataaatatcaaaccttctcacctaaaattttgagcaaaacttctcactaaaacttgtggactaaacccgaaaatcttcagaaaattaatatttagtactggaaaagtcagattaaatgtctgcacaagactttctattgttgggaataaacaaattaatattggattaaaatagttgaaatatttaggtaaaatgctacatgtcatttattcattggaaattatttcttaaaatttaaaattcattatttaaaagttatttttgaaaaagggccgatttctgaaattccttctaagatagggtctttctagacgtaaagttgatctattaaattttaaatttatcttaaattcttacaaagtaagtattaatctttgtttcctttactattcatttacattttgtcctatatttcgtttaaaatatgtcatatattaaaaaaaattaaaaattttactttctttttttacgttatagctatatgacgtgacaaaaaatcaactttcacatgcgcctagtagacttcaagttaaaaggggataatggatactttgcctatcttttaccatatattttaaaatccttaattattaagttttccaatatctctcaccattcattttctcctatcatatattttaggagtccttaataattaagtttactaataaactttattatatattataggactcctcaattattagttctctttatgtctctcatcgtacattttcctcttgtcttatttgttaggacacttgaaattttcaaaatatattttgcttttaatatatgaagttgtgtttgattgtagtttttgtaaatatatttaattttttgaatttttattttctaaaagaaacataaaatttaaaagatttaaaagtatcattaaactattagaaataatatatctatgttgttaaaaatgatggttcttaattaactgttttattataaaatatcagataattcgttttatttacgcaaaagttaagtgaagtaacgaaattataaatcccatagaatattgtgtatatacttggcacatgatgattgtaacatccttaattattattaattcatcgaacctattatttcttcattgtctatgtacatttatccttaataattccacttcaggatttattagttctttggttattggtttaagtttattttacaaccaagtgaattgaatttgtcctccattaatatttattggattaaaaaataaataaatttgctcttatttgtagaaagatttagacttttaaaatattacgttttctgactcttttcttatcaaaattggactctctcacttccacaaaacttaattacatgaacaatatcattagggaaagctt

SEQ ID NO: 4 - DNA, 13908 bp, synthetic construct
  source 1..13908: mol_type = other DNA
agcttgcatgcctgcaggtcgactctagaggatcgacttcaaccttattagtgaatggacaataaaggttataagctcctttactgtgaaagcccaccagtaacatcaccttgcttatatcattcagcttctttctagtaacatttggaacgtgtttataacagaaaaaaacccaaaaactctgaaaagactcacacttttcttatctccagtccacctctcaaaaggaacaatttccttcagcttcttggttggacacctgttgagcacatatgctgcagtggcaacagtttctccccacaaagtgttaggaagcttcttctccttcagcatgttccttgtcatatcaagcaaagttcggttttcaacaagaccattatgttgaggagtatatggatcagtcacttcatgctcaattccattctctttacagaacttcttgaactctgtagagttatactcacctccaccatcagttctgagaatcttcagaagtctgaccactttatttctcagccttgattatgaatttcttaaattcagcaaacacctcgtgtttgaattttataagggatacccatgtcatccttgtgaactcatccataaatgacataaagtattattccctcctagtgaaaggtttgtaatgggccacacacataagaatgcactactcctaaagcatgttttgctctttgagctacttttgatgaaaatggcagtcttggttgcttccctttcatgcacacattacatgacttttttggtttcttaattgtaggaattccacgtaccagtttctttgaattcaaattccctaagctcctaaagttcaaatgaccaaatcttttgttccacaactcactttccttcacaacacttgttgcgctaaggcattcagagtctgcagttttaacattcgccttgaatgttttactccttccatgttctgactccataatcaacttctgataacagtcatacagcttcaaaagaatgtcattcatggtaactggaaatcccttttcaattaattgacctacactcatcagattgctcttcatgccaagaacgtaccaagacgttctgaattaatgcagattttctattattcataatcactctaacattccccattcctttagcatttagttacttatcatcagcacatctaatcttggttttcttcctagagtcaaaatcaaccagccatttcttatttccagtatgatggtttgaacaaccagtgtccatatatcaccagtcttctatagacgcactatcataactagaagccattaatagcacatgttcatcatggtgctcagatccttagaatgttcaattgctacaacgatgtaatcaaactgatgagtaagagatctaagtaccttctcaatgatactttcctcataaagagtttctccatgcgacttcatctcatttgtgatcagaatcactctagagatgtagtcagataacttctcattgttcttcatgcttagattctcatactgctcacgtagagactgaagtttcaccttctacactgatgcatcactatcgtagcaccacaccagtctgtctcacacaaccttttccgtcattgaatcaacgattttcttaaacacgttcacatccacacactgatggatgtagaacaacgcattctgatccttcttcctcatatcacactgagcatttctttgcgcatccgttgcattttctagaagtgaagcataaacttcgttgatgagatcaagaacatcttgagcaccaaataacacacacatctgaatcatccaacgattccagttgttgtcgtcgaacaatggnagcntggtgcacagattcacaacgatatattataanttttgttttatgaaatttaagaacaaatttccattattcttaaaatgtttacacactgatgtagactgcaaaaggaataaagatacaatttgttcacaccactcacttgcgtaaatataagtgagagttaatgagaaatactaaaataccctctaaaattatgaattaattctaacaatctctaatgttagtataatccattaaacactttgatggcaggtataacaagggtgtaagttagtgtatacatattaggctcttattatttttatattatctctgcttttcttcttcatgttctcactaatatgatattatctcccttccctaaattatttatatttattagaaaaagagtttcattttttaaaaatatattaccgtaatttttcaaaaaataaaatttaaatatattttataaaaaaattatttaataatttatttacattaatgcataaatataaataaatactgtcatttaatatttaaccttttaacaataaattatatttatttaattcaactaatataagctaagttatctcatccaaccaattaaaaagatcatttgaaaatacctttttatttagtttgtggcggtttcaactgtcaaaaaaaaggaatttttacgacgatataaatttaaaccagcaaaaaattgaagcagttaagcgaaccaactcatggtatgtggatatatttatctttgtcgtttatatcggattcgaatctctataatgatgaaaaattaatatcaaactttaaataagaacgtcatttatagagccattttgggaaacacatatttcatgtacacgtgattcgcaaatttccaataactctatatatagccctcctcagtttcatgcatttgctcacaacataaccttccttgaatctggaaaacgtcacattgcttccgcatatcgggtcagcaacggctaaaatccgcttgaatatgttcacacaagccgctcaaaacatgattgacgccgtatacggaagaacgccgaaaaaccttactaaggaatttcaataagaagaaaaatcccggttggttcagccggggtttatttttcgctagataaaaagtactatttttaaattctttctattcctttctttcgttgctgatacaatgaaaaggaatcagcttcacatgatgaaaatgggaggtattgctttgaaaaaacgattatcgtggatttccgtttgtttactggtgcttgtctccgcggcggggatgctgttttcaacagctgccaaaacggaaacatcttctcacaaggcacacacagaagcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataacgaaaaaaacggcttccctgcggaggccgtttttttcagctttacataaagtgtgtaataaatttttcttcaaactctgatcggtcaatttcactttgctagccaccaccaccaccaccacgtgtgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccgggtacgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaaagctttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctaccactttgtacaagaaagctgggtttaagccttcttgatagcagcagcgagttcatcgtaagcaacttcccaagccctagacaattcatctgaccatttatcaccaacagcagctttaatagttttaagcaaagcttctttcacaacaacgaactgaggatctgtaacagccttttgagcatgaactgaacccaaagcagcatcagcaacaactgtcccagaagctttaagctgaccagctgaatctctcaccagagcaaaaagcttttctgcatgtccagtcaacttaggatttgttggatcaacaccgttagccaaaaatgaaaacaaatccttagcagcaggagctttttccaaaattgatgtgtagaaaaccacagaatattgtggaatatttgccttgaaagcttcaaatgatgatgaaacaagagcatcttgcttttcagtaaaagcaaccatggtggcagcctgcttttttgtacaaacttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggccaacttttctatacaaagttg

SEQ ID NO: 5 - DNA, 435 bp, Glycine max
  source 1..435: mol_type = genomic DNA
atggttgcttttactgaaaagcaagatgctcttgtttcatcatcatttgaagctttcaaggcaaatattccacaatattctgtggttttctacacatcaattttggaaaaagctcctgctgctaaggatttgttttcatttttggctaacggtgttgatccaacaaatcctaagttgactggacatgcagaaaagctttttgctctggtgagagattcagctggtcagcttaaagcttctgggacagttgttgctgatgctgctttgggttcagttcatgctcaaaaggctgttacagatcctcagttcgttgttgtgaaagaagctttgcttaaaactattaaagctgctgttggtgataaatggtcagatgaattgtctagggcttgggaagttgcttacgatgaactcgctgctgctatcaagaaggcttaa

SEQ ID NO: 6 - DNA, 333 bp, Bacillus amyloliquefaciens
  source 1..333: mol_type = genomic DNA
gcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataa

SEQ ID NO: 7 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 8 - DNA, 2023 bp, synthetic construct
  source 1..2023: mol_type = other DNA
atggtagatctgagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgacgactcgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactcgaagtcacagccaaaagccagacagagtctgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttcagctgtctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaa

SEQ ID NO: 9 - DNA, 677 bp, Cauliflower mosaic virus
  source 1..677: mol_type = genomic DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 10 - DNA, 2203 bp, Solanum lycopersicum
  source 1..2203: mol_type = genomic DNA
aagctttccctaatgatattgttcatgtaattaagttttgtggaagtgagagagtccaattttgataagaaaagagtcagaaaacgtaatattttaaaagtctaaatctttctacaaataagagcaaatttatttattttttaatccaataaatattaatggaggacaaattcaattcacttggttgtaaaataaacttaaaccaataaccaaagaactaataaatcctgaagtggaattattaaggataaatgtacatagacaatgaagaaataataggttcgatgaattaataataattaaggatgttacaatcatcatgtgccaagtatatacacaatattctatgggatttataatttcgttacttcacttaacttttgcgtaaataaaacgaattatctgatattttataataaaacagttaattaagaaccatcatttttaacaacatagatatattatttctaatagtttaatgatacttttaaatcttttaaattttatgtttcttttagaaaataaaaattcaaaaaattaaatatatttacaaaaactacaatcaaacacaacttcatatattaaaagcaaaatatattttgaaaatttcaagtgtcctaacaaataagacaagaggaaaatgtacgatgagagacataaagagaactaataattgaggagtcctataatatataataaagtttattagtaaacttaattattaaggactcctaaaatatatgataggagaaaatgaatggtgagagatattggaaaacttaataattaaggattttaaaatatatggtaaaagataggcaaagtatccattatccccttttaacttgaagtctactaggcgcatgtgaaagttgattttttgtcacgtcatatagctataacgtaaaaaaagaaagtaaaatttttaattttttttaatatatgacatattttaaacgaaatataggacaaaatgtaaatgaatagtaaaggaaacaaagattaatacttactttgtaagaatttaagataaatttaaaatttaatagatcaactttacgtctagaaagaccctatcttagaaggaatttcagaaatcggccctttttcaaaaataacttttaaataatgaattttaaattttaagaaataatttccaatgaataaatgacatgtagcattttacctaaatatttcaactattttaatccaatattaatttgtttattcccaacaatagaaagtcttgtgcagacatttaatctgacttttccagtactaaatattaattttctgaagattttcgggtttagtccacaagttttagtgagaagttttgctcaaaattttaggtgagaaggtttgatatttatcttttgttaaattaatttatctaggtgactattatttatttaagtagaaattcatatcattacttttgccaacttgtagtcataataggagtaggtgtatatgatgaaggaataaacaagttcagtgaagtgattaaaataaaatataatttaggtgtacatcaaataaaaaccttaaagtttagaaaggcaccgaataattttgcatagaagatattagtaaatttataaaaataaaagaaatgtagttgtcaagttgtcttcttttttttggataaaaatagcagttggcttatgtcattcttttacaacctccatgccacttgtccaattgttgacacttaactaattagtttgattcatgtatgaatactaaataattttttaggactgactcaaatatttttatattatcatagtaatatttatctaatttttaggaccacttattactaaataataaattaactactactatattattgttgtgaaacaacaacgttttggttgttatgatgaaacgtacactatatcagtatgaaaaattcaaaacgattagtataaattatattgaaaatttgatatttttctattcttaatcagacgtattgggtttcatattttaaaaagggactaaacttagaagagaagtttgtttgaaactacttttgtctctttcttgttcccatttctctcttagatttcaaaaagtgaactactttatctctttctttgttcacattttattttattctattataaatatggcatcctcatattgagatttttagaaattattctaatcattcacagtgcaaaagaagggatcc

SEQ ID NO: 11 - DNA, 2736 bp, Pisum sativum
  source 1..2736: mol_type = genomic DNA
gacttcaaccttattagtgaatggacaataaaggttataagctcctttactgtgaaagcccaccagtaacatcaccttgcttatatcattcagcttctttctagtaacatttggaacgtgtttataacagaaaaaaacccaaaaactctgaaaagactcacacttttcttatctccagtccacctctcaaaaggaacaatttccttcagcttcttggttggacacctgttgagcacatatgctgcagtggcaacagtttctccccacaaagtgttaggaagcttcttctccttcagcatgttccttgtcatatcaagcaaagttcggttttcaacaagaccattatgttgaggagtatatggatcagtcacttcatgctcaattccattctctttacagaacttcttgaactctgtagagttatactcacctccaccatcagttctgagaatcttcagaagtctgaccactttatttctcagccttgattatgaatttcttaaattcagcaaacacctcgtgtttgaattttataagggatacccatgtcatccttgtgaactcatccataaatgacataaagtattattccctcctagtgaaaggtttgtaatgggccacacacataagaatgcactactcctaaagcatgttttgctctttgagctacttttgatgaaaatggcagtcttggttgcttccctttcatgcacacattacatgacttttttggtttcttaattgtaggaattccacgtaccagtttctttgaattcaaattccctaagctcctaaagttcaaatgaccaaatcttttgttccacaactcactttccttcacaacacttgttgcgctaaggcattcagagtctgcagttttaacattcgccttgaatgttttactccttccatgttctgactccataatcaacttctgataacagtcatacagcttcaaaagaatgtcattcatggtaactggaaatcccttttcaattaattgacctacactcatcagattgctcttcatgccaagaacgtaccaagacgttctgaattaatgcagattttctattattcataatcactctaacattccccattcctttagcatttagttacttatcatcagcacatctaatcttggttttcttcctagagtcaaaatcaaccagccatttcttatttccagtatgatggtttgaacaaccagtgtccatatatcaccagtcttctatagacgcactatcataactagaagccattaatagcacatgttcatcatggtgctcagatccttagaatgttcaattgctacaacgatgtaatcaaactgatgagtaagagatctaagtaccttctcaatgatactttcctcataaagagtttctccatgcgacttcatctcatttgtgatcagaatcactctagagatgtagtcagataacttctcattgttcttcatgcttagattctcatactgctcacgtagagactgaagtttcaccttctacactgatgcatcactatcgtagcaccacaccagtctgtctcacacaaccttttccgtcattgaatcaacgattttcttaaacacgttcacatccacacactgatggatgtagaacaacgcattctgatccttcttcctcatatcacactgagcatttctttgcgcatccgttgcattttctagaagtgaagcataaacttcgttgatgagatcaagaacatcttgagcaccaaataacacacacatctgaatcatccaacgattccagttgttgtcgtcgaacaatggnagcntggtgcacagattcacaacgatatattataanttttgttttatgaaatttaagaacaaatttccattattcttaaaatgtttacacactgatgtagactgcaaaaggaataaagatacaatttgttcacaccactcacttgcgtaaatataagtgagagttaatgagaaatactaaaataccctctaaaattatgaattaattctaacaatctctaatgttagtataatccattaaacactttgatggcaggtataacaagggtgtaagttagtgtatacatattaggctcttattatttttatattatctctgcttttcttcttcatgttctcactaatatgatattatctcccttccctaaattatttatatttattagaaaaagagtttcattttttaaaaatatattaccgtaatttttcaaaaaataaaatttaaatatattttataaaaaaattatttaataatttatttacattaatgcataaatataaataaatactgtcatttaatatttaaccttttaacaataaattatatttatttaattcaactaatataagctaagttatctcatccaaccaattaaaaagatcatttgaaaatacctttttatttagtttgtggcggtttcaactgtcaaaaaaaaggaatttttacgacgatataaatttaaaccagcaaaaaattgaagcagttaagcgaaccaactcatggtatgtggatatatttatctttgtcgtttatatcggattcgaatctctataatgatgaaaaattaatatcaaactttaaataagaacgtcatttatagagccattttgggaaacacatatttcatgtacacgtgattcgcaaatttccaataactctatatatagccctcctcagtttcatgcatttgctcacaacataaccttccttgaat

SEQ ID NO: 12 - DNA, 864 bp, Nicotiana tabacum
  source 1..864: mol_type = genomic DNA
ctttttggttagcgaatgcaattaatttagacattgtgttatgttccagttaaccgcttccctgcacttctttcaatctatctctcgatagaaaattgtgatactttgcgacttctatcagaggactttttgttttccatgtaacaatctgtcattttcgatggggagatttgcacaaataggctatttatgtgtcccaatttaaattttaaccccatgtcgatcagaacttagccacgagcaccagaagtttgatggatatgtgactttgtcactatccggtttactaatcaagagctatttttattcaaaattggatatctagctaagtataactggataatttgcattaacagattgaatatagtgccaaacaagaagggacaattgacttgtcactttatgaaagatgattcaaacatgattttttatgtactaatatatacatcctactcgaattaaagcgacataggctcgaagtatgcacatttagcaatgtaaattaaatcagtttttgaatcaagctaaaagcagacttgcataaggtgggtggctggactagaataaacatcttctctagcacagcttcataatgtaatttccataactgaaatcagggtgagacaaaattttggtactttttcctcacactaagtccatgtttgcaacaaattaatacatgaaaccttaatgttaccctcagattagcctgctactccccattttcctcgaaatgctccaacaaaagttagttttgcaagttgttgtgtatgtcttgtgctctatatatgcccttgtggtgcaagtgtaacagtacaacatcatcactcaaatcaaagtttttacttaaagaaattagctaaa

SEQ ID NO: 13 - DNA, 253 bp, unidentified
  source 1..253: mol_type = genomic DNA; note = Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 14 - DNA, 175 bp, Cauliflower mosaic virus
  source 1..175: mol_type = genomic DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 15 - DNA, 25 bp, unidentified
  source 1..25: mol_type = genomic DNA; note = Agrobacterium tumefaciens
gtttacaccacaatatatcctgcca

SEQ ID NO: 16 - DNA, 25 bp, unidentified
  source 1..25: mol_type = genomic DNA; note = Agrobacterium tumefaciens
gtttacccgccaatatatcctgtca

(19) United States
(12) Patent Application Publication
(10) Pub. No.: US 2026/0125693 A1
(43) Pub. Date: May 7, 2026

(54) GENETICALLY ENGINEERED VEGETABLES

(71) Applicant: Impossible Vines Inc., Miami Beach, FL (US)

(72) Inventors: Gabriella Rothberg, Miami Beach, FL (US); Jonathan M. Rothberg, Miami Beach, FL (US)

(73) Assignee: Impossible Vines Inc., Miami Beach, FL (US)

(21) Appl. No.: 19/376,424

(22) Filed: Oct. 31, 2025

Related U.S. Application Data
(60) Provisional application No. 63/715,207, filed on Nov. 1, 2024.

(51) Int. Cl. C12N 15/82; C07K 14/32; C07K 14/415

(52) U.S. Cl. CPC C12N 15/8205; C07K 14/32; C07K 14/415; C12N 15/8209; C12N 15/8257; C12N 15/8289

(57) ABSTRACT

The present disclosure relates at least in part to genetic engineering of vegetable (e.g., tomato) plants to, for example, produce heme, a molecule contributing to a meat-like flavor, and to the incorporation of male sterility in these plants to facilitate the production of F1 hybrid seeds. Described herein, in some embodiments, are methods for constructing and introducing into such plants genetic constructs containing both a heme gene and a male sterility gene, the resulting plants, and the use of these plants in hybrid seed production. The present disclosure also provides at least in part methods for enhancing lycopene content in plants, such as tomato plants, either through genetic modification or selective breeding. While introducing key genes like Psy is effective, adding multiple genes or knocking out competing pathways can lead to more substantial increases in lycopene levels. Enhanced lycopene content can provide added health benefits, including potential cancer protection, making these plants, such as tomatoes, particularly valuable in the context of functional foods. The plants may be of varieties with high lycopene content combined with genetic modifications as provided herein. The present disclosure offers a comprehensive solution for producing plants (e.g., tomatoes) with increased nutritional value.

### CROSS REFERENCE TO RELATED APPLICATIONS

This application claims the benefit of priority under 35 U.S.C. § 119 (e) to U.S. Provisional Application No. 63/715,207, filed Nov. 1, 2024, the entire contents of which are herein incorporated by reference.

### REFERENCE TO AN ELECTRONIC SEQUENCE LISTING

The contents of the electronic sequence listing (1063470000US02-SEQ-KVC.xml; Size: 77,300 bytes; and Date of Creation: Oct. 31, 2025) are herein incorporated by reference in their entirety.

### FIELD

The present disclosure generally relates, at least in part, to the field of plant biotechnology and genetic engineering. More specifically, it relates to the production of genetically engineered vegetable plants, such as tomato (Solanum lycopersicum) plants and eggplant (Solanum melongena) plants, engineered to express a protein to enhance iron content and contribute to a meat-like flavor, optionally combined with a male sterility gene to facilitate F1 hybrid seed production. The present disclosure also relates to increasing lycopene production, such as in tomato plants, to, in some embodiments, enhance its health benefits and antioxidant properties.

### SUMMARY

Vegetables are widely recognized for their nutritional and health benefits, and the global demand for plant-based foods has continued to increase in response to growing consumer interest in sustainability and wellness. Tomatoes are among the most extensively cultivated vegetables and serve as an important dietary source of vitamins and antioxidants. In particular, the naturally occurring carotenoid, lycopene, imparts the characteristic red color and has health-promoting properties, including potential roles in reducing cancer risk and supporting cardiovascular and skin health. Iron and associated carrier molecules, such as heme, contribute to the characteristic meat-like flavor of many foods. The iron content in tomatoes, however, is inherently low, limiting their contribution to dietary iron intake.

Advances in plant genetic engineering can enable the precise enhancement of nutritional and sensory compounds in crops, offering an efficient and sustainable means to improve food quality, reduce agricultural inputs, and align with global trends towards plant-based diets. Accordingly, developing genetically improved tomato plant varieties that exhibit enhanced lycopene accumulation and/or elevated iron levels provides a promising opportunity to deliver greater nutritional benefits to the world's expanding population.

Aspects of the present disclosure relate, at least in part, to genetically engineered plants (e.g., vegetable plants such as tomato plants and eggplant plants) that can exhibit enhanced nutritional and/or sensory properties through the introduction of genes coding for heme production and, optionally, male sterility to enable controlled F1 hybrid seed production. The present disclosure further encompasses strategies for enhancing lycopene biosynthesis through genetic modification to yield plants (e.g., vegetable plants such as tomato plants) that can produce fruit (e.g., tomatoes) with improved antioxidant capacity.

Aspects of the present disclosure further provide genetically engineered vegetable plants that combine enhanced nutritional composition with improved hybridization efficiency. In some embodiments, the present disclosure relates to tomatoes or eggplants engineered to express a protein to produce heme, an iron-carrier molecule contributing to a meat-like flavor, while also introducing a male-sterility gene to facilitate efficient and controlled F1 hybrid seeds. The integration of these two traits enables the development of hybrid tomato or eggplant varieties that deliver desirable flavors without the need for manual emasculation during breeding.

Aspects of the present disclosure provides methods for enhancing lycopene biosynthesis in vegetable fruit, such as tomato fruit, through the overexpression of key enzymes involved in its biosynthesis, such as phytoene synthase (Psy). In some embodiments, the present disclosure relates to the use of gene-editing technologies, such as CRISPR/Cas9-mediated technology, to downregulate competing pathways, thereby reducing precursor flux toward lycopene production and achieving elevated antioxidant content.

The present disclosure thus can integrate flavor enhancement, nutritional fortification, and reproductive control into a single transgenic platform. This combination allows for sustainable, high-value vegetable cultivation that aligns with consumer demands for nutritious, plant-based foods, with improved sensory traits such as meatier tastes. Furthermore, the present disclosure provides the corresponding genetic constructs, transformation methods, and resulting plants, seeds, and fruits produced thereby.

Accordingly, aspects of the present disclosure relate to a genetic construct or set of genetic constructs comprising a first promoter operably linked to a first coding sequence and, optionally, a second promoter operably linked to a second coding sequence, wherein the first coding sequence encodes a heme-producing gene, and wherein the second coding sequence encodes a male-sterility gene. In some embodiments of any one of the compositions or methods provided herein, the first and second promoter are the same promoter or are different promoters that can result in the expression of the first and second coding sequence.

In some embodiments of any one of the compositions or methods provided herein, the genetic construct or set of genetic constructs comprises any one of the nucleotide sequences provided herein, such as a nucleotide sequence that is at least 70%, at least 75%, at least 80%, at least 85%, at least 90%, at least 95%, or at least 98% identical to SEQ ID NO: 3. In some embodiments of any one of the compositions or methods provided herein, the genetic construct or set of genetic constructs comprises the nucleotide sequence of SEQ ID NO: 3.

In some embodiments of any one of the compositions or methods provided herein, the genetic construct or set of genetic constructs comprises a nucleotide sequence that is at least 70%, at least 75%, at least 80%, at least 85%, at least 90%, at least 95%, or at least 98% identical to SEQ ID NO: 4. In some embodiments of any one of the compositions or methods provided herein, the genetic construct or set of genetic constructs comprises the nucleotide sequence of SEQ ID NO: 4.

In some embodiments of any one of the compositions or methods provided herein, the heme-producing gene is any one of the heme-producing genes provided herein, such as a leghemoglobin gene derived from Glycine max. In some embodiments of any one of the compositions or methods provided herein, a leghemoglobin gene comprises a nucleotide sequence that is at least 70%, at least 75%, at least 80%, at least 85%, at least 90%, at least 95%, or at least 98% identical to SEQ ID NO: 5. In some embodiments of any one of the compositions or methods provided herein, a leghemoglobin gene comprises the nucleotide sequence of SEQ ID NO: 5.

In some embodiments of any one of the compositions or methods provided herein, the male-sterility gene is any one of the male-sterility genes provided herein, such as a barnase gene derived from Bacillus amyloliquefaciens. In some embodiments of any one of the compositions or methods provided herein, a barnase gene comprises a nucleotide sequence that is at least 70%, at least 75%, at least 80%, at least 85%, at least 90%, at least 95%, or at least 98% identical to SEQ ID NO: 6. In some embodiments of any one of the compositions or methods provided herein, a barnase gene comprises the nucleotide sequence of SEQ ID NO: 6.

In some embodiments of any one of the compositions or methods provided herein, the first and/or second promoter is any one of the promoters provided herein. In some embodiments of any one of the compositions or methods provided herein, the first promoter is a constitutive promoter. In some embodiments of any one of the compositions or methods provided herein, the second promoter is a constitutive promoter. In some embodiments of any one of the compositions or methods provided herein, the constitutive promoter comprises a nucleotide sequence that is at least 70%, at least 75%, at least 80%, at least 85%, at least 90%, at least 95%, or at least 98% identical to SEQ ID NO: 9. In some embodiments of any one of the compositions or methods provided herein, the constitutive promoter comprises the nucleotide sequence of SEQ ID NO: 9.

In some embodiments of any one of the compositions or methods provided herein, the first promoter is a plant promoter. In some embodiments of any one of the compositions or methods provided herein, the first promoter is a plant-specific promoter. In some embodiments of any one of the compositions or methods provided herein, the first promoter is a fruit-specific promoter. In some embodiments of any one of the compositions or methods provided herein, the first promoter comprises a nucleotide sequence that is at least 70%, at least 75%, at least 80%, at least 85%, at least 90%, at least 95%, or at least 98% identical to SEQ ID NO: 10. In some embodiments of any one of the compositions or methods provided herein, the first promoter comprises the nucleotide sequence of SEQ ID NO: 10. In some embodiments of any one of the compositions or methods provided herein, the first promoter comprises a nucleotide sequence that is at least 70%, at least 75%, at least 80%, at least 85%, at least 90%, at least 95%, or at least 98% identical to SEQ ID NO: 11. In some embodiments of any one of the compositions or methods provided herein, the first promoter comprises the nucleotide sequence of SEQ ID NO: 11. In some embodiments of any one of the compositions or methods provided herein, the first promoter comprises a nucleotide sequence that is at least 70%, at least 75%, at least 80%, at least 85%, at least 90%, at least 95%, or at least 98% identical to SEQ ID NO: 12. In some embodiments of any one of the compositions or methods provided herein, the first promoter comprises the nucleotide sequence of SEQ ID NO: 12. In some embodiments of any one of the compositions or methods provided herein, the first promoter and/or the second promoter is a plant promoter. In some embodiments of any one of the compositions or methods provided herein, the plant promoter comprises the nucleotide sequence of any one of SEQ ID NOs: 10-12.

In some embodiments of any one of the compositions or methods provided herein, the first promoter and the second promoter are the same. In some embodiments of any one of the compositions or methods provided herein, the first promoter and the second promoter are different. In some embodiments of any one of the compositions or methods provided herein, the promoter controls expression of one or more genes. In some embodiments of any one of the compositions or methods provided herein, the promoter controls expression of two genes. In some embodiments of any one of the compositions or methods provided herein, the first and second promoter are the same single promoter in the genetic construct or set of genetic constructs.

In some embodiments of any one of the compositions or methods provided herein, the genetic construct or set of genetic constructs further comprises a terminator sequence. In some embodiments of any one of the compositions or methods provided herein, the terminator sequence is any one of such sequences provided herein. In some embodiments of any one of the compositions or methods provided herein, the terminator sequence comprises the nucleotide sequence of SEQ ID NO: 13 or SEQ ID NO: 14. In some embodiments of any one of the compositions or methods provided herein, the terminator sequence comprises a nucleotide sequence that is at least 70%, at least 75%, at least 80%, at least 85%, at least 90%, at least 95%, or at least 98% identical to SEQ ID NO: 13. In some embodiments of any one of the compositions or methods provided herein, the terminator sequence comprises the nucleotide sequence of SEQ ID NO: 13. In some embodiments of any one of the compositions or methods provided herein, the terminator sequence comprises a nucleotide sequence that is at least 70%, at least 75%, at least 80%, at least 85%, at least 90%, at least 95%, or at least 98% identical to SEQ ID NO: 14. In some embodiments of any one of the compositions or methods provided herein, the terminator sequence comprises the nucleotide sequence of SEQ ID NO: 14.

In some embodiments of any one of the compositions or methods provided herein, the genetic construct or set of genetic constructs further comprises a selectable marker gene and/or reporter gene and/or an antibiotic resistance gene. In some embodiments of any one of the compositions or methods provided herein, the selectable marker gene and/or reporter gene and/or antibiotic resistance gene is any one such genes provided herein. In some embodiments of any one of the compositions or methods provided herein, the antibiotic resistance gene is a hygromycin resistance gene. In some embodiments of any one of the compositions or methods provided herein, the hygromycin resistance gene comprises a nucleotide sequence that is at least 70%, at least 75%, at least 80%, at least 85%, at least 90%, at least 95%, or at least 98% identical to SEQ ID NO: 7. In some embodiments of any one of the compositions or methods provided herein, the hygromycin resistance gene comprises the nucleotide sequence of SEQ ID NO: 7.

In some embodiments of any one of the compositions or methods provided herein, the genetic construct or set of genetic constructs further comprises a first flanking sequence and/or a second flanking sequence. In some embodiments of any one of the compositions or methods provided herein, the first flanking sequence and/or a second flanking sequence is any one of such sequences provided herein. In some embodiments of any one of the compositions or methods provided herein, the first flanking sequence comprises the nucleotide sequence of SEQ ID NO: 15. In some embodiments of any one of the compositions or methods provided herein, the second flanking sequence comprises the nucleotide sequence of SEQ ID NO: 16. In some embodiments of any one of the compositions or methods provided herein, the first flanking sequence comprises a nucleotide sequence that is at least 70%, at least 75%, at least 80%, at least 85%, at least 90%, at least 95%, or at least 98% identical to SEQ ID NO: 15. In some embodiments of any one of the compositions or methods provided herein, the first flanking sequence comprises the nucleotide sequence of SEQ ID NO: 15. In some embodiments of any one of the compositions or methods provided herein, the second flanking sequence comprises a nucleotide sequence that is at least 70%, at least 75%, at least 80%, at least 85%, at least 90%, at least 95%, or at least 98% identical to SEQ ID NO: 16. In some embodiments of any one of the compositions or methods provided herein, the second flanking sequence comprises the nucleotide sequence of SEQ ID NO: 16.

Aspects of the present disclosure relate to a genetic construct or set of genetic constructs comprising a first promoter (e.g., fruit specific promoter such as the E8 promoter) operably linked to a first coding sequence, wherein the first coding sequence encodes a phytoene synthase (Psy) gene. In some embodiments of any one of the compositions or methods provided herein, in addition to the genetic construct or set of genetic constructs also comprised are means for knocking out genes that compete with the lycopene biosynthesis pathway, such as any one of the gene editing systems provided herein or any one or more or combination of components thereof. Aspects of the present disclosure relate to a plant cell (e.g., vegetable plant cell) comprising any genetic construct or set of genetic constructs provided herein. In some embodiments of any one of the compositions or methods provided herein, the plant cell is tomato plant cell or an eggplant plant cell. In some embodiments of any one of the compositions or methods provided herein, the plant or plant cell is of a variety with a naturally elevated lycopene content such as Tangerine, Double Rich, or Health Kick.

Aspects of the present disclosure relate to a method, the method comprising transforming a plant cell (e.g., vegetable plant cell) with any genetic construct or set of genetic constructs provided herein. In some embodiments of any one of the compositions or methods provided herein, transforming is via any one of such methods provided herein such as via Agrobacterium-mediated transformation. In some embodiments of any one of the compositions or methods provided herein, the plant cell is tomato plant cell or an eggplant plant cell.

Aspects of the present disclosure relate to a method of producing a genetically engineered plant (e.g., vegetable plant), the method comprising introducing into a plant any genetic construct or set of genetic constructs provided herein. In some embodiments of any one of the methods provided herein, the introducing is via any one of such methods provided herein, such as via Agrobacterium-mediated transformation. In some embodiments of any one of the compositions or methods provided herein, the genetically engineered plant is a tomato plant or an eggplant plant.

Aspects of the present disclosure relate to a method of producing an F1 hybrid vegetable plant (e.g., tomato plant or eggplant plant), the method comprising: crossing any genetically engineered plant described herein with a male-fertile vegetable plant (e.g., tomato plant or eggplant plant) to produce a crossed plant; harvesting seeds from the crossed plant; and growing the F1 hybrid tomato plant from the seeds.

### BRIEF DESCRIPTION OF THE DRAWINGS

The following drawings form part of the present specification and are included to further demonstrate certain aspects of the present disclosure, which may be better understood by reference to one or more of these drawings in combination with the detailed description of specific embodiments presented in this disclosure. The accompanying drawings are not intended to be drawn to scale. The drawings are illustrative only and are not required for enablement of the disclosure. For purposes of clarity, not every component may be labeled in every drawing. In the drawings:

FIG. 1 is a block diagram illustrating elements of a system according to embodiments of the present disclosure.

FIG. 2 is a flow diagram illustrating steps of an engineering process according to embodiments of the present disclosure.

FIG. 3 is a plasmid map depicting an exemplary dual-cassette genetic construct comprising an expression cassette encoding the heme protein under the control of a constitutive promoter and an expression cassette encoding the barnase gene under an anther-specific promoter.

FIG. 4 is a plasmid map depicting a control plasmid comprising a β-glucuronidase (GUS) reporter gene and a hygromycin resistance gene to ensure effective gene uptake for Agrobacterium-mediated transformation.

FIG. 5 is a plasmid map depicting a codon-optimized leghemoglobin gene under control of a Cauliflower Mosaic Virus (CaMV 35S) promoter and a Noplaine Synthase (NOS) terminator, with hygromycin resistance gene under control of a CaMV 35S promoter and a CaMV polyadenylation (polyA) termination signal.

FIG. 6 is a plasmid map depicting a codon-optimized leghemoglobin gene under control of an E8S promoter and a NOS terminator, a barnase gene under control of a Pisum sativum Endothecium 1 (PSEND1) promoter and a NOS terminator, and a hygromycin resistance gene under control of a CaMV 35S promoter and a CaMV polyA termination signal.

FIG. 7 is a plasmid map depicting a codon-optimized leghemoglobin gene under control of a CaMV 35S promoter and a NOS terminator, a barnase gene under control of a PSEND1 promoter and a NOS terminator, and a hygromycin resistance gene under control of a CaMV 35S promoter and a CaMV polyA termination signal.

FIG. 8 shows microscope images depicting optimized conditions for tomato regeneration in control cotyledons with callus formation in 4 week cotyledon explants. Arrows point at early callus, characterized by translucent tissue that accumulates on the adaxial side of leaf.

### DETAILED DESCRIPTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes. Such incorporation by reference is not intended to be an admission that any of the incorporated publications, patents and patent applications cited herein constitute prior art.

As used in this specification and the appended claims, the singular forms “a,” “an” and “the” include plural referents unless the content clearly dictates otherwise. For example, reference to “a molecule” includes a mixture of two or more such molecules or a plurality of such molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps. In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

The following description is provided to enable any person skilled in the art of plant biotechnology to make and use the invention of the present disclosure and sets forth the best modes contemplated by the inventors for carrying out the invention of the present disclosure. It is understood that variations in the described embodiments may be made without departing from the scope of the present disclosure.

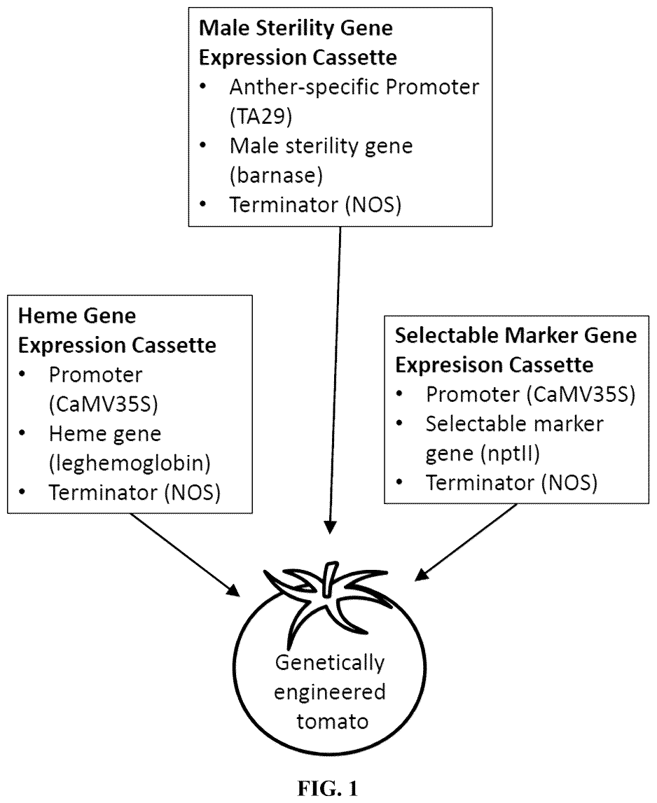

Accordingly, aspects of the present disclosure relate to a genetically engineered vegetable plant that (i) produces heme, contributing to a meat-like flavor, and is (ii) male-sterile, facilitating the production of F1 hybrid seeds (FIG. 1). Aspects of the present disclosure also relate to a genetic construct or set of genetic constructs comprising a heme gene expression cassette, including a promoter, the leghemoglobin gene (or a functional homolog or equivalent), and a terminator; a male sterility gene expression cassette, including an anther-specific promoter, the barnase gene (or a functional homolog or equivalent), and a terminator; and, optionally, a selectable marker and/or reporter and/or antibiotic resistance gene expression cassette.

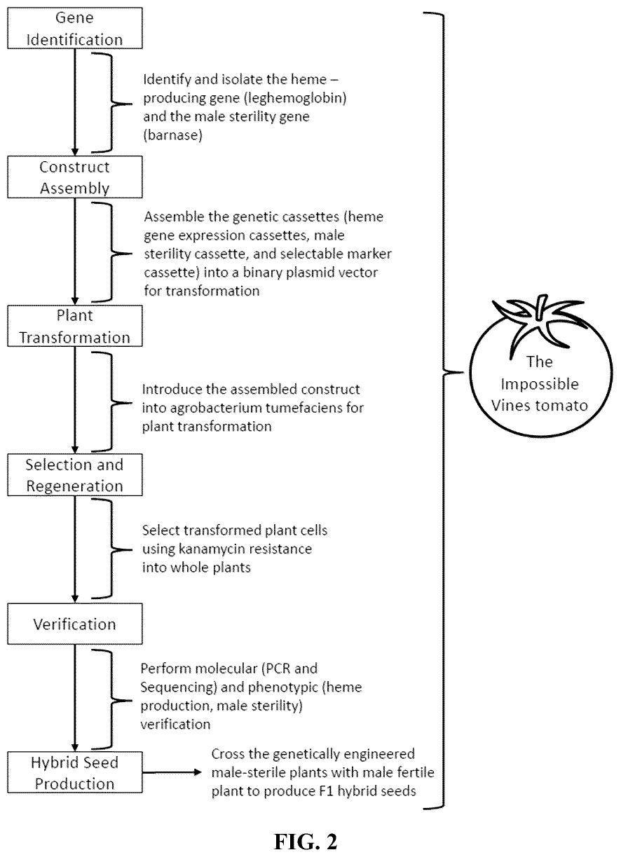

Further aspects of the present disclosure relate to a method for producing transgenic plants (e.g., vegetable plants) by Agrobacterium-mediated transformation using cotyledons, followed by selection, shoot regeneration, and genomic validation of the desired traits. FIG. 2 is a flow diagram illustrating steps of an engineering process according to an embodiment of the present disclosure.

Further aspects of the present disclosure relate to a method for increasing lycopene content in tomatoes through upregulation of the biosynthesis pathway, under a fruit-specific promoter, and synergistic downregulation of competing pathways.

Further aspects of the present disclosure relate to use of male sterile plants (e.g., vegetable plants such as tomato plants or eggplant plants) as Parental Line A in controlled cross-pollination with male-fertile Parental Line B to produce F1 hybrid seeds exhibiting the described genetically modified traits.

Further aspects of the present disclosure relate to F1 hybrid plants (e.g., vegetable plants such as tomato plants or eggplant plants) and fruits produced by the foregoing methods, characterized by enhanced lycopene accumulation, increased heme and iron levels, desirable flavor attributes, and/or overall improved nutritional value.

### Genetic Constructs

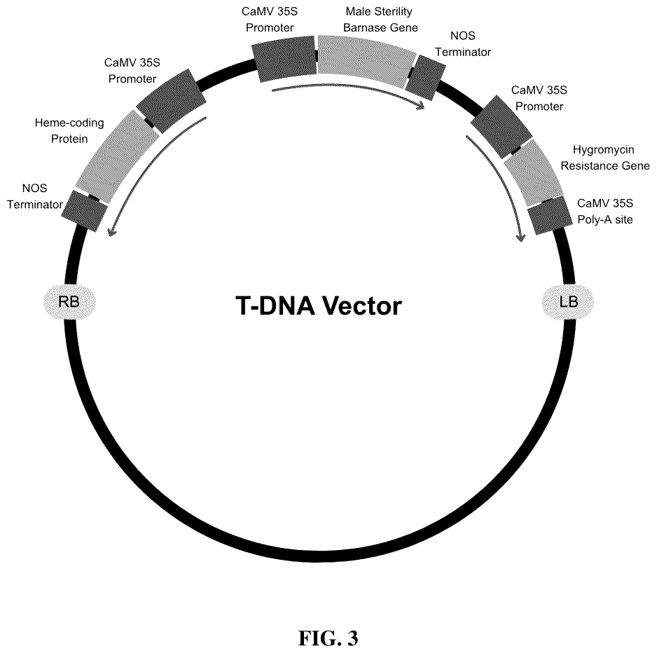

Aspects of the present disclosure relate to genetic constructs useful for expressing one or more genes in a plant (i.e., a genetically engineered plant). The term “genetic construct,” as used herein, includes synthetic deoxyribonucleic acid (DNA) molecules that contain specific genetic information intended for manipulation or expression in a target organism. Genetic constructs typically include several components, such as coding sequences (genes), promoters, enhancers and regulatory elements, selectable markers, and terminator sequences. A non-limiting example of a genetic construct described herein is shown in FIG. 3. In an embodiment, the genetic constructs are vectors such as for introducing the one or more genes into a plant or plant cell as provided herein. The term “vector,” as used herein, includes DNA molecules used to carry foreign genetic material into another cell, serving as a vehicle for delivering DNA. In some embodiments, a genetic construct is a plasmid. The term “plasmid,” as used herein, includes small, circular DNA molecules used to introduce specific genes into host cells.

In some embodiments, a genetic construct of the present disclosure delivers a single gene to a cell. In some embodiments, a genetic construct of the present disclosure delivers two or more genes to a cell. In some embodiments, a genetic construct of the present disclosure is a dual-gene genetic construct.

In some embodiments, a set of genetic constructs of the present disclosure delivers two or more genes to a cell.

### Vectors

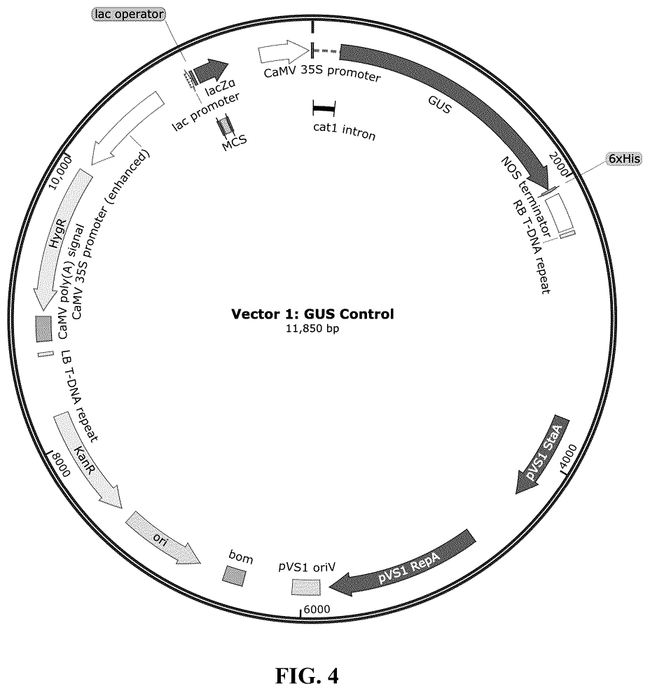

Aspects of the present disclosure relate to genetic constructs as vectors for delivering one or more genes to a plant cell (e.g., a tomato cell or an eggplant cell). In some embodiments, a vector of the present disclosure is useful for ensuring effective gene uptake by a plant or plant cell via transformation (e.g., Agrobacterium-mediated transformation). A non-limiting example of a vector comprising a β-glucuronidase (GUS) reporter gene flanked by a Cauliflower Mosaic Virus (CaMV 35S) promoter and a Noplaine Synthase (NOS) terminator, a hygromycin resistance gene under control of a CaMV 35S promoter and a CaMV polyadenylation (polyA) termination signal, and a left border (LB) T-DNA repeat and a right border (RB) T-DNA repeat is shown in FIG. 4 and provided as SEQ ID NO: 1:

(SEQ ID NO: 1)
GATCTGAGGGTAAATTTCTAGTTTTTCTCCTTCATTTTCTTGGTTAGGACCCTTTTCTCTTTTTATTTTTTTGAGCT
TTGATCTTTCTTTAAACTGATCTATTTTTTAATTGATTGGTTATGGTGTAAATATTACATAGCTTTAACTGATAATC
TGATTACTTTATTTCGTGTGTCTATGATGATGATGATAGTTACAGAACCGACGACTCGTCCGTCCTGTAGAAACCCC
AACCCGTGAAATCAAAAAACTCGACGGCCTGTGGGCATTCAGTCTGGATCGCGAAAACTGTGGAATTGATCAGCGTT
GGTGGGAAAGCGCGTTACAAGAAAGCCGGGCAATTGCTGTGCCAGGCAGTTTTAACGATCAGTTCGCCGATGCAGAT
ATTCGTAATTATGCGGGCAACGTCTGGTATCAGCGCGAAGTCTTTATACCGAAAGGTTGGGCAGGCCAGCGTATCGT
GCTGCGTTTCGATGCGGTCACTCATTACGGCAAAGTGTGGGTCAATAATCAGGAAGTGATGGAGCATCAGGGCGGCT
ATACGCCATTTGAAGCCGATGTCACGCCGTATGTTATTGCCGGGAAAAGTGTACGTATCACCGTTTGTGTGAACAAC
GAACTGAACTGGCAGACTATCCCGCCGGGAATGGTGATTACCGACGAAAACGGCAAGAAAAAGCAGTCTTACTTCCA
TGATTTCTTTAACTATGCCGGAATCCATCGCAGCGTAATGCTCTACACCACGCCGAACACCTGGGTGGACGATATCA
CCGTGGTGACGCATGTCGCGCAAGACTGTAACCACGCGTCTGTTGACTGGCAGGTGGTGGCCAATGGTGATGTCAGC
GTTGAACTGCGTGATGCGGATCAACAGGTGGTTGCAACTGGACAAGGCACTAGCGGGACTTTGCAAGTGGTGAATCC
GCACCTCTGGCAACCGGGTGAAGGTTATCTCTATGAACTCGAAGTCACAGCCAAAAGCCAGACAGAGTCTGATATCT
ACCCGCTTCGCGTCGGCATCCGGTCAGTGGCAGTGAAGGGCCAACAGTTCCTGATTAACCACAAACCGTTCTACTTT
ACTGGCTTTGGTCGTCATGAAGATGCGGACTTACGTGGCAAAGGATTCGATAACGTGCTGATGGTGCACGACCACGC
ATTAATGGACTGGATTGGGGCCAACTCCTACCGTACCTCGCATTACCCTTACGCTGAAGAGATGCTCGACTGGGCAG
ATGAACATGGCATCGTGGTGATTGATGAAACTGCTGCTGTCGGCTTTCAGCTGTCTTTAGGCATTGGTTTCGAAGCG
GGCAACAAGCCGAAAGAACTGTACAGCGAAGAGGCAGTCAACGGGGAAACTCAGCAAGCGCACTTACAGGCGATTAA
AGAGCTGATAGCGCGTGACAAAAACCACCCAAGCGTGGTGATGTGGAGTATTGCCAACGAACCGGATACCCGTCCGC
AAGGTGCACGGGAATATTTCGCGCCACTGGCGGAAGCAACGCGTAAACTCGACCCGACGCGTCCGATCACCTGCGTC
AATGTAATGTTCTGCGACGCTCACACCGATACCATCAGCGATCTCTTTGATGTGCTGTGCCTGAACCGTTATTACGG
ATGGTATGTCCAAAGCGGCGATTTGGAAACGGCAGAGAAGGTACTGGAAAAAGAACTTCTGGCCTGGCAGGAGAAAC
TGCATCAGCCGATTATCATCACCGAATACGGCGTGGATACGTTAGCCGGGCTGCACTCAATGTACACCGACATGTGG
AGTGAAGAGTATCAGTGTGCATGGCTGGATATGTATCACCGCGTCTTTGATCGCGTCAGCGCCGTCGTCGGTGAACA
GGTATGGAATTTCGCCGATTTTGCGACCTCGCAAGGCATATTGCGCGTTGGCGGTAACAAGAAAGGGATCTTCACTC
GCGACCGCAAACCGAAGTCGGCGGCTTTTCTGCTGCAAAAACGCTGGACTGGCATGAACTTCGGTGAAAAACCGCAG
CAGGGAGGCAAACAAGCTAGCCACCACCACCACCACCACGTGTGAATTACAGGTGACCAGCTCGAATTTCCCCGATC
GTTCAAACATTTGGCAATAAAGTTTCTTAAGATTGAATCCTGTTGCCGGTCTTGCGATGATTATCATATAATTTCTG
TTGAATTACGTTAAGCATGTAATAATTAACATGTAATGCATGACGTTATTTATGAGATGGGTTTTTATGATTAGAGT
CCCGCAATTATACATTTAATACGCGATAGAAAACAAAATATAGCGCGCAAACTAGGATAAATTATCGCGCGCGGTGT
CATCTATGTTACTAGATCGGGAATTAAACTATCAGTGTTTGACAGGATATATTGGCGGGTAAACCTAAGAGAAAAGA
GCGTTTATTAGAATAACGGATATTTAAAAGGGCGTGAAAAGGTTTATCCGTTCGTCCATTTGTATGTGCATGCCAAC
CACAGGGTTCCCCTCGGGATCAAAGTACTTTGATCCAACCCCTCCGCTGCTATAGTGCAGTCGGCTTCTGACGTTCA
GTGCAGCCGTCTTCTGAAAACGACATGTCGCACAAGTCCTAAGTTACGCGACAGGCTGCCGCCCTGCCCTTTTCCTG
GCGTTTTCTTGTCGCGTGTTTTAGTCGCATAAAGTAGAATACTTGCGACTAGAACCGGAGACATTACGCCATGAACA
AGAGCGCCGCCGCTGGCCTGCTGGGCTATGCCCGCGTCAGCACCGACGACCAGGACTTGACCAACCAACGGGCCGAA
CTGCACGCGGCCGGCTGCACCAAGCTGTTTTCCGAGAAGATCACCGGCACCAGGCGCGACCGCCCGGAGCTGGCCAG
GATGCTTGACCACCTACGCCCTGGCGACGTTGTGACAGTGACCAGGCTAGACCGCCTGGCCCGCAGCACCCGCGACC
TACTGGACATTGCCGAGCGCATCCAGGAGGCCGGCGCGGGCCTGCGTAGCCTGGCAGAGCCGTGGGCCGACACCACC
ACGCCGGCCGGCCGCATGGTGTTGACCGTGTTCGCCGGCATTGCCGAGTTCGAGCGTTCCCTAATCATCGACCGCAC
CCGGAGCGGGCGCGAGGCCGCCAAGGCCCGAGGCGTGAAGTTTGGCCCCCGCCCTACCCTCACCCCGGCACAGATCG
CGCACGCCCGCGAGCTGATCGACCAGGAAGGCCGCACCGTGAAAGAGGCGGCTGCACTGCTTGGCGTGCATCGCTCG
ACCCTGTACCGCGCACTTGAGCGCAGCGAGGAAGTGACGCCCACCGAGGCCAGGCGGCGCGGTGCCTTCCGTGAGGA
CGCATTGACCGAGGCCGACGCCCTGGCGGCCGCCGAGAATGAACGCCAAGAGGAACAAGCATGAAACCGCACCAGGA
CGGCCAGGACGAACCGTTTTTCATTACCGAAGAGATCGAGGCGGAGATGATCGCGGCCGGGTACGTGTTCGAGCCGC
CCGCGCACGTCTCAACCGTGCGGCTGCATGAAATCCTGGCCGGTTTGTCTGATGCCAAGCTGGCGGCCTGGCCGGCC
AGCTTGGCCGCTGAAGAAACCGAGCGCCGCCGTCTAAAAAGGTGATGTGTATTTGAGTAAAACAGCTTGCGTCATGC
GGTCGCTGCGTATATGATGCGATGAGTAAATAAACAAATACGCAAGGGGAACGCATGAAGGTTATCGCTGTACTTAA
CCAGAAAGGCGGGTCAGGCAAGACGACCATCGCAACCCATCTAGCCCGCGCCCTGCAACTCGCCGGGGCCGATGTTC
TGTTAGTCGATTCCGATCCCCAGGGCAGTGCCCGCGATTGGGCGGCCGTGCGGGAAGATCAACCGCTAACCGTTGTC
GGCATCGACCGCCCGACGATTGACCGCGACGTGAAGGCCATCGGCCGGCGCGACTTCGTAGTGATCGACGGAGCGCC
CCAGGCGGCGGACTTGGCTGTGTCCGCGATCAAGGCAGCCGACTTCGTGCTGATTCCGGTGCAGCCAAGCCCTTACG
ACATATGGGCCACCGCCGACCTGGTGGAGCTGGTTAAGCAGCGCATTGAGGTCACGGATGGAAGGCTACAAGCGGCC
TTTGTCGTGTCGCGGGCGATCAAAGGCACGCGCATCGGCGGTGAGGTTGCCGAGGCGCTGGCCGGGTACGAGCTGCC
CATTCTTGAGTCCCGTATCACGCAGCGCGTGAGCTACCCAGGCACTGCCGCCGCCGGCACAACCGTTCTTGAATCAG
AACCCGAGGGCGACGCTGCCCGCGAGGTCCAGGCGCTGGCCGCTGAAATTAAATCAAAACTCATTTGAGTTAATGAG
GTAAAGAGAAAATGAGCAAAAGCACAAACACGCTAAGTGCCGGCCGTCCGAGCGCACGCAGCAGCAAGGCTGCAACG
TTGGCCAGCCTGGCAGACACGCCAGCCATGAAGCGGGTCAACTTTCAGTTGCCGGCGGAGGATCACACCAAGCTGAA
GATGTACGCGGTACGCCAAGGCAAGACCATTACCGAGCTGCTATCTGAATACATCGCGCAGCTACCAGAGTAAATGA
GCAAATGAATAAATGAGTAGATGAATTTTAGCGGCTAAAGGAGGCGGCATGGAAAATCAAGAACAACCAGGCACCGA
CGCCGTGGAATGCCCCATGTGTGGAGGAACGGGCGGTTGGCCAGGCGTAAGCGGCTGGGTTGTCTGCCGGCCCTGCA
ATGGCACTGGAACCCCCAAGCCCGAGGAATCGGCGTGAGCGGTCGCAAACCATCCGGCCCGGTACAAATCGGCGCGG
CGCTGGGTGATGACCTGGTGGAGAAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAACCGCCGGCAGCCGGTGCGCCGTC
GATTAGGAAGCCGCCCAAGGGCGACGAGCAACCAGATTTTTTCGTTCCGATGCTCTATGACGTGGGCACCCGCGATA
GTCGCAGCATCATGGACGTGGCCGTTTTCCGTCTGTCGAAGCGTGACCGACGAGCTGGCGAGGTGATCCGCTACGAG
CTTCCAGACGGGCACGTAGAGGTTTCCGCAGGGCCGGCCGGCATGGCCAGTGTGTGGGATTACGACCTGGTACTGAT
GGCGGTTTCCCATCTAACCGAATCCATGAACCGATACCGGGAAGGGAAGGGAGACAAGCCCGGCCGCGTGTTCCGTC
CACACGTTGCGGACGTACTCAAGTTCTGCCGGCGAGCCGATGGCGGAAAGCAGAAAGACGACCTGGTAGAAACCTGC
ATTCGGTTAAACACCACGCACGTTGCCATGCAGCGTACGAAGAAGGCCAAGAACGGCCGCCTGGTGACGGTATCCGA
GGGTGAAGCCTTGATTAGCCGCTACAAGATCGTAAAGAGCGAAACCGGGCGGCCGGAGTACATCGAGATCGAGCTAG
CTGATTGGATGTACCGCGAGATCACAGAAGGCAAGAACCCGGACGTGCTGACGGTTCACCCCGATTACTTTTTGATC
GATCCCGGCATCGGCCGTTTTCTCTACCGCCTGGCACGCCGCGCCGCAGGCAAGGCAGAAGCCAGATGGTTGTTCAA
GACGATCTACGAACGCAGTGGCAGCGCCGGAGAGTTCAAGAAGTTCTGTTTCACCGTGCGCAAGCTGATCGGGTCAA
ATGACCTGCCGGAGTACGATTTGAAGGAGGAGGCGGGGCAGGCTGGCCCGATCCTAGTCATGCGCTACCGCAACCTG
ATCGAGGGCGAAGCATCCGCCGGTTCCTAATGTACGGAGCAGATGCTAGGGCAAATTGCCCTAGCAGGGGAAAAAGG
TCGAAAAGGTCTCTTTCCTGTGGATAGCACGTACATTGGGAACCCAAAGCCGTACATTGGGAACCGGAACCCGTACA
TTGGGAACCCAAAGCCGTACATTGGGAACCGGTCACACATGTAAGTGACTGATATAAAAGAGAAAAAAGGCGATTTT
TCCGCCTAAAACTCTTTAAAACTTATTAAAACTCTTAAAACCCGCCTGGCCTGTGCATAACTGTCTGGCCAGCGCAC
AGCCGAAGAGCTGCAAAAAGCGCCTACCCTTCGGTCGCTGCGCTCCCTACGCCCCGCCGCTTCGCGTCGGCCTATCG
CGGCCGCTGGCCGCTCAAAAATGGCTGGCCTACGGCCAGGCAATCTACCAGGGCGCGGACAAGCCGCGCCGTCGCCA
CTCGACCGCCGGCGCCCACATCAAGGCACCCTGCCTCGCGCGTTTCGGTGATGACGGTGAAAACCTCTGACACATGC
AGCTCCCGGAGACGGTCACAGCTTGTCTGTAAGCGGATGCCGGGAGCAGACAAGCCCGTCAGGGCGCGTCAGCGGGT
GTTGGCGGGTGTCGGGGCGCAGCCATGACCCAGTCACGTAGCGATAGCGGAGTGTATACTGGCTTAACTATGCGGCA
TCAGAGCAGATTGTACTGAGAGTGCACCATATGCGGTGTGAAATACCGCACAGATGCGTAAGGAGAAAATACCGCAT
CAGGCGCTCTTCCGCTTCCTCGCTCACTGACTCGCTGCGCTCGGTCGTTCGGCTGCGGCGAGCGGTATCAGCTCACT
CAAAGGCGGTAATACGGTTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCGCTTTCTCAT
AGCTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGCCTAACTAC
GGCTACACTAGAAGGACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCACGTTAAGGGATTTTG
GTCATGCATTCTAGGTACTAAAACAATTCATCCAGTAAAATATAATATTTTATTTTCTCCCAATCAGGCTTGATCCC
CAGTAAGTCAAAAAATAGCTCGACATACTGTTCTTCCCCGATATCCTCCCTGATCGACCGGACGCAGAAGGCAATGT
CATACCACTTGTCCGCCCTGCCGCTTCTCCCAAGATCAATAAAGCCACTTACTTTGCCATCTTTCACAAAGATGTTG
CTGTCTCCCAGGTCGCCGTGGGAAAAGACAAGTTCCTCTTCGGGCTTTTCCGTCTTTAAAAAATCATACAGCTCGCG
CGGATCTTTAAATGGAGTGTCTTCTTCCCAGTTTTCGCAATCCACATCGGCCAGATCGTTATTCAGTAAGTAATCCA
ATTCGGCTAAGCGGCTGTCTAAGCTATTCGTATAGGGACAATCCGATATGTCGATGGAGTGAAAGAGCCTGATGCAC
TCCGCATACAGCTCGATAATCTTTTCAGGGCTTTGTTCATCTTCATACTCTTCCGAGCAAAGGACGCCATCGGCCTC
ACTCATGAGCAGATTGCTCCAGCCATCATGCCGTTCAAAGTGCAGGACCTTTGGAACAGGCAGCTTTCCTTCCAGCC
ATAGCATCATGTCCTTTTCCCGTTCCACATCATAGGTGGTCCCTTTATACCGGCTGTCCGTCATTTTTAAATATAGG
TTTTCATTTTCTCCCACCAGCTTATATACCTTAGCAGGAGACATTCCTTCCGTATCTTTTACGCAGCGGTATTTTTC
GATCAGTTTTTTCAATTCCGGTGATATTCTCATTTTAGCCATTTATTATTTCCTTCCTCTTTTCTACAGTATTTAAA
GATACCCCAAGAAGCTAATTATAACAAGACGAACTCCAATTCACTGTTCCTTGCATTCTAAAACCTTAAATACCAGA
AAACAGCTTTTTCAAAGTTGTTTTCAAAGTTGGCGTATAACATAGTATCGACGGAGCCGATTTTGAAACCGCGGTGA
TCACAGGCAGCAACGCTCTGTCATCGTTACAATCAACATGCTACCCTCCGCGAGATCATCCGTGTTTCAAACCCGGC
AGCTTAGTTGCCGTTCTTCCGAATAGCATCGGTAACATGAGCAAAGTCTGCCGCCTTACAACGGCTCTCCCGCTGAC
GCCGTCCCGGACTGATGGGCTGCCTGTATCGAGTGGTGATTTTGTGCCGAGCTGCCGGTCGGGGAGCTGTTGGCTGG
CTGGTGGCAGGATATATTGTGGTGTAAACAAATTGACGCTTAGACAACTTAATAACACATTGCGGACGTTTTTAATG
TACTGAATTAACGCCGAATTAATTCGGGGGATCTGGATTTTAGTACTGGATTTTGGTTTTAGGAATTAGAAATTTTA
TTGATAGAAGTATTTTACAAATACAAATACATACTAAGGGTTTCTTATATGCTCAACACATGAGCGAAACCCTATAG
GAACCCTAATTCCCTTATCTGGGAACTACTCACACATTATTATGGAGAAACTCGAGCTTGTCGATCGACAGATCCGG
TCGGCATCTACTCTATTTCTTTGCCCTCGGACGAGTGCTGGGGCGTCGGTTTCCACTATCGGCGAGTACTTCTACAC
AGCCATCGGTCCAGACGGCCGCGCTTCTGCGGGCGATTTGTGTACGCCCGACAGTCCCGGCTCCGGATCGGACGATT
GCGTCGCATCGACCCTGCGCCCAAGCTGCATCATCGAAATTGCCGTCAACCAAGCTCTGATAGAGTTGGTCAAGACC
AATGCGGAGCATATACGCCCGGAGTCGTGGCGATCCTGCAAGCTCCGGATGCCTCCGCTCGAAGTAGCGCGTCTGCT
GCTCCATACAAGCCAACCACGGCCTCCAGAAGAAGATGTTGGCGACCTCGTATTGGGAATCCCCGAACATCGCCTCG
CTCCAGTCAATGACCGCTGTTATGCGGCCATTGTCCGTCAGGACATTGTTGGAGCCGAAATCCGCGTGCACGAGGTG
CCGGACTTCGGGGCAGTCCTCGGCCCAAAGCATCAGCTCATCGAGAGCCTGCGCGACGGACGCACTGACGGTGTCGT
CCATCACAGTTTGCCAGTGATACACATGGGGATCAGCAATCGCGCATATGAAATCACGCCATGTAGTGTATTGACCG
ATTCCTTGCGGTCCGAATGGGCCGAACCCGCTCGTCTGGCTAAGATCGGCCGCAGCGATCGCATCCATAGCCTCCGC
GACCGGTTGTAGAACAGCGGGCAGTTCGGTTTCAGGCAGGTCTTGCAACGTGACACCCTGTGCACGGCGGGAGATGC
AATAGGTCAGGCTCTCGCTAAACTCCCCAATGTCAAGCACTTCCGGAATCGGGAGCGCGGCCGATGCAAAGTGCCGA
TAAACATAACGATCTTTGTAGAAACCATCGGCGCAGCTATTTACCCGCAGGACATATCCACGCCCTCCTACATCGAA
GCTGAAAGCACGAGATTCTTCGCCCTCCGAGAGCTGCATCAGGTCGGAGACGCTGTCGAACTTTTCGATCAGAAACT
TCTCGACAGACGTCGCGGTGAGTTCAGGCTTTTTCATATCTCATTGCCCCCCGGGATCTGCGAAAGCTCGAGAGAGA
TAGATTTGTAGAGAGAGACTGGTGATTTCAGCGTGTCCTCTCCAAATGAAATGAACTTCCTTATATAGAGGAAGGTC
TTGCGAAGGATAGTGGGATTGTGCGTCATCCCTTACGTCAGTGGAGATATCACATCAATCCACTTGCTTTGAAGACG
TGGTTGGAACGTCTTCTTTTTCCACGATGCTCCTCGTGGGTGGGGGTCCATCTTTGGGACCACTGTCGGCAGAGGCA
TCTTGAACGATAGCCTTTCCTTTATCGCAATGATGGCATTTGTAGGTGCCACCTTCCTTTTCTACTGTCCTTTTGAT
GAAGTGACAGATAGCTGGGCAATGGAATCCGAGGAGGTTTCCCGATATTACCCTTTGTTGAAAAGTCTCAATAGCCC
TTTGGTCTTCTGAGACTGTATCTTTGATATTCTTGGAGTAGACGAGAGTGTCGTGCTCCACCATGTTATCACATCAA
TCCACTTGCTTTGAAGACGTGGTTGGAACGTCTTCTTTTTCCACGATGCTCCTCGTGGGTGGGGGTCCATCTTTGGG
ACCACTGTCGGCAGAGGCATCTTGAACGATAGCCTTTCCTTTATCGCAATGATGGCATTTGTAGGTGCCACCTTCCT
TTTCTACTGTCCTTTTGATGAAGTGACAGATAGCTGGGCAATGGAATCCGAGGAGGTTTCCCGATATTACCCTTTGT
TGAAAAGTCTCAATAGCCCTTTGGTCTTCTGAGACTGTATCTTTGATATTCTTGGAGTAGACGAGAGTGTCGTGCTC
CACCATGTTGGCAAGCTGCTCTAGCCAATACGCAAACCGCCTCTCCCCGCGCGTTGGCCGATTCATTAATGCAGCTG
GCACGACAGGTTTCCCGACTGGAAAGCGGGCAGTGAGCGCAACGCAATTAATGTGAGTTAGCTCACTCATTAGGCAC
CCCAGGCTTTACACTTTATGCTTCCGGCTCGTATGTTGTGTGGAATTGTGAGCGGATAACAATTTCACACAGGAAAC
AGCTATGACCATGATTACGAATTCGAGCTCGGTACCCGGGGATCCTCTAGAGTCGACCTGCAGGCATGCAAGCTTGG
CACTGGCCGTCGTTTTACAACGTCGTGACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCCCAACAGTTGCGCAGCCTGAATGGCGA
ATGCTAGAGCAGCTTGAGCTTGGATCAGATTGTCGTTTCCCGCCTTCAGTTTAGCTTCATGGAGTCAAAGATTCAAA
TAGAGGACCTAACAGAACTCGCCGTAAAGACTGGCGAACAGTTCATACAGAGTCTCTTACGACTCAATGACAAGAAG
AAAATCTTCGTCAACATGGTGGAGCACGACACACTTGTCTACTCCAAAAATATCAAAGATACAGTCTCAGAAGACCA
AAGGGCAATTGAGACTTTTCAACAAAGGGTAATATCCGGAAACCTCCTCGGATTCCATTGCCCAGCTATCTGTCACT
TTATTGTGAAGATAGTGGAAAAGGAAGGTGGCTCCTACAAATGCCATCATTGCGATAAAGGAAAGGCCATCGTTGAA
GATGCCTCTGCCGACAGTGGTCCCAAAGATGGACCCCCACCCACGAGGAGCATCGTGGAAAAAGAAGACGTTCCAAC
CACGTCTTCAAAGCAAGTGGATTGATGTGATATCTCCACTGACGTAAGGGATGACGCACAATCCCACTATCCTTCGC
AAGACCCTTCCTCTATATAAGGAAGTTCATTTCATTTGGAGAGAACACGGGGGACTCTTGACCATGGTA

In some embodiments, a vector of the present disclosure (e.g., a plasmid) is useful for delivering a heme-producing gene to a plant cell (e.g., a tomato cell or an eggplant cell).

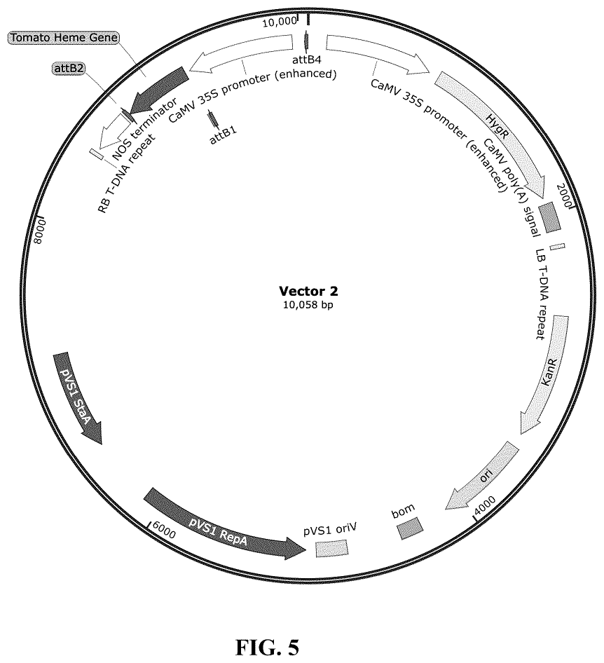

A non-limiting example of a vector comprising a codon-optimized heme-producing gene flanked by a CaMV 35S promoter and a NOS terminator, a hygromycin resistance gene under control of a CaMV 35S promoter and a CaMV polyA termination signal, and a left border (LB) T-DNA repeat and a right border (RB) T-DNA repeat is shown in FIG. 5 and provided as SEQ ID NO: 2:

(SEQ ID NO: 2)
AGCTTGCATGCCTGCAGGTCGACTCTAGAGGATCCCCGGGTACGCCAACATGGTGGAGCACGACACTCTCGTCTACT
CCAAGAATATCAAAGATACAGTCTCAGAAGACCAAAGGGCTATTGAGACTTTTCAACAAAGGGTAATATCGGGAAAC
CTCCTCGGATTCCATTGCCCAGCTATCTGTCACTTCATCAAAAGGACAGTAGAAAAGGAAGGTGGCACCTACAAATG
CCATCATTGCGATAAAGGAAAGGCTATCGTTCAAGATGCCTCTGCCGACAGTGGTCCCAAAGATGGACCCCCACCCA
CGAGGAGCATCGTGGAAAAAGAAGACGTTCCAACCACGTCTTCAAAGCAAGTGGATTGATGTGAACATGGTGGAGCA
CGACACTCTCGTCTACTCCAAGAATATCAAAGATACAGTCTCAGAAGACCAAAGGGCTATTGAGACTTTTCAACAAA
GGGTAATATCGGGAAACCTCCTCGGATTCCATTGCCCAGCTATCTGTCACTTCATCAAAAGGACAGTAGAAAAGGAA
GGTGGCACCTACAAATGCCATCATTGCGATAAAGGAAAGGCTATCGTTCAAGATGCCTCTGCCGACAGTGGTCCCAA
AGATGGACCCCCACCCACGAGGAGCATCGTGGAAAAAGAAGACGTTCCAACCACGTCTTCAAAGCAAGTGGATTGAT
GTGATATCTCCACTGACGTAAGGGATGACGCACAATCCCACTATCCTTCGCAAGACCCTTCCTCTATATAAGGAAGT
TCATTTCATTTGGAGAGGACACGCTGAAATCACCAGTCTCTCTCTACAAATCTATCTCTCTCGAGCTTTCGCAGATC
CGGGGGGCAATGAGATATGAAAAAGCCTGAACTCACCGCGACGTCTGTCGAGAAGTTTCTGATCGAAAAGTTCGACA
GCGTCTCCGACCTGATGCAGCTCTCGGAGGGCGAAGAATCTCGTGCTTTCAGCTTCGATGTAGGAGGGCGTGGATAT
GTCCTGCGGGTAAATAGCTGCGCCGATGGTTTCTACAAAGATCGTTATGTTTATCGGCACTTTGCATCGGCCGCGCT
CCCGATTCCGGAAGTGCTTGACATTGGGGAGTTTAGCGAGAGCCTGACCTATTGCATCTCCCGCCGTTCACAGGGTG
TCACGTTGCAAGACCTGCCTGAAACCGAACTGCCCGCTGTTCTACAACCGGTCGCGGAGGCTATGGATGCGATCGCT
GCGGCCGATCTTAGCCAGACGAGCGGGTTCGGCCCATTCGGACCGCAAGGAATCGGTCAATACACTACATGGCGTGA
TTTCATATGCGCGATTGCTGATCCCCATGTGTATCACTGGCAAACTGTGATGGACGACACCGTCAGTGCGTCCGTCG
CGCAGGCTCTCGATGAGCTGATGCTTTGGGCCGAGGACTGCCCCGAAGTCCGGCACCTCGTGCACGCGGATTTCGGC
TCCAACAATGTCCTGACGGACAATGGCCGCATAACAGCGGTCATTGACTGGAGCGAGGCGATGTTCGGGGATTCCCA
ATACGAGGTCGCCAACATCTTCTTCTGGAGGCCGTGGTTGGCTTGTATGGAGCAGCAGACGCGCTACTTCGAGCGGA
GGCATCCGGAGCTTGCAGGATCGCCACGACTCCGGGCGTATATGCTCCGCATTGGTCTTGACCAACTCTATCAGAGC
TTGGTTGACGGCAATTTCGATGATGCAGCTTGGGCGCAGGGTCGATGCGACGCAATCGTCCGATCCGGAGCCGGGAC
TGTCGGGCGTACACAAATCGCCCGCAGAAGCGCGGCCGTCTGGACCGATGGCTGTGTAGAAGTACTCGCCGATAGTG
GAAACCGACGCCCCAGCACTCGTCCGAGGGCAAAGAAATAGAGTAGATGCCGACCGGGATCTGTCGATCGACAAGCT
CGAGTTTCTCCATAATAATGTGTGAGTAGTTCCCAGATAAGGGAATTAGGGTTCCTATAGGGTTTCGCTCATGTGTT
GAGCATATAAGAAACCCTTAGTATGTATTTGTATTTGTAAAATACTTCTATCAATAAAATTTCTAATTCCTAAAACC
AAAATCCAGTACTAAAATCCAGATCCCCCGGTACCGAGCTCGAATTCAATTCGGCGTTAATTCAGTACATTAAAAAC
GTCCGCAATGTGTTATTAAGTTGTCTAAGCGTCAATTTGTTTACACCACAATATATCCTGCCACCAGCCAGCCAACA
GCTCCCCGACCGGCAGCTCGGCACAAAATCACCACTCGATACAGGCAGCCCATCAGTCCGGGACGGCGTCAGCGGGA
GAGCCGTTGTAAGGCGGCAGACTTTGCTCATGTTACCGATGCTATTCGGAAGAACGGCAACTAAGCTGCCGGGTTTG
AAACACGGATGATCTCGCGGAGGGTAGCATGTTGATTGTAACGATGACAGAGCGTTGCTGCCTGTGATCACCGCGGT
TTCAAAATCGGCTCCGTCGATACTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCTTCTTGGGGTATCTTTAAATACT
GTAGAAAAGAGGAAGGAAATAATAAATGGCTAAAATGAGAATATCACCGGAATTGAAAAAACTGATCGAAAAATACC
GCTGCGTAAAAGATACGGAAGGAATGTCTCCTGCTAAGGTATATAAGCTGGTGGGAGAAAATGAAAACCTATATTTA
AAAATGACGGACAGCCGGTATAAAGGGACCACCTATGATGTGGAACGGGAAAAGGACATGATGCTATGGCTGGAAGG
AAAGCTGCCTGTTCCAAAGGTCCTGCACTTTGAACGGCATGATGGCTGGAGCAATCTGCTCATGAGTGAGGCCGATG
GCGTCCTTTGCTCGGAAGAGTATGAAGATGAACAAAGCCCTGAAAAGATTATCGAGCTGTATGCGGAGTGCATCAGG
CTCTTTCACTCCATCGACATATCGGATTGTCCCTATACGAATAGCTTAGACAGCCGCTTAGCCGAATTGGATTACTT
ACTGAATAACGATCTGGCCGATGTGGATTGCGAAAACTGGGAAGAAGACACTCCATTTAAAGATCCGCGCGAGCTGT
ATGATTTTTTAAAGACGGAAAAGCCCGAAGAGGAACTTGTCTTTTCCCACGGCGACCTGGGAGACAGCAACATCTTT
GTGAAAGATGGCAAAGTAAGTGGCTTTATTGATCTTGGGAGAAGCGGCAGGGCGGACAAGTGGTATGACATTGCCTT
CTGCGTCCGGTCGATCAGGGAGGATATCGGGGAAGAACAGTATGTCGAGCTATTTTTTGACTTACTGGGGATCAAGC
CTGATTGGGAGAAAATAAAATATTATATTTTACTGGATGAATTGTTTTAGTACCTAGAATGCATGACCAAAATCCCT
TAACGTGAGTTTTCGTTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCTAGTGTAGCCGTAGTTAGGC
CACCACTTCAAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGGAAGCGGAAGAGCGCCTGATGCGGTATT
TTCTCCTTACGCATCTGTGCGGTATTTCACACCGCATATGGTGCACTCTCAGTACAATCTGCTCTGATGCCGCATAG
TTAAGCCAGTATACACTCCGCTATCGCTACGTGACTGGGTCATGGCTGCGCCCCGACACCCGCCAACACCCGCTGAC
GCGCCCTGACGGGCTTGTCTGCTCCCGGCATCCGCTTACAGACAAGCTGTGACCGTCTCCGGGAGCTGCATGTGTCA
GAGGTTTTCACCGTCATCACCGAAACGCGCGAGGCAGGGTGCCTTGATGTGGGCGCCGGCGGTCGAGTGGCGACGGC
GCGGCTTGTCCGCGCCCTGGTAGATTGCCTGGCCGTAGGCCAGCCATTTTTGAGCGGCCAGCGGCCGCGATAGGCCG
ACGCGAAGCGGCGGGGCGTAGGGAGCGCAGCGACCGAAGGGTAGGCGCTTTTTGCAGCTCTTCGGCTGTGCGCTGGC
CAGACAGTTATGCACAGGCCAGGCGGGTTTTAAGAGTTTTAATAAGTTTTAAAGAGTTTTAGGCGGAAAAATCGCCT
TTTTTCTCTTTTATATCAGTCACTTACATGTGTGACCGGTTCCCAATGTACGGCTTTGGGTTCCCAATGTACGGGTT
CCGGTTCCCAATGTACGGCTTTGGGTTCCCAATGTACGTGCTATCCACAGGAAAGAGACCTTTTCGACCTTTTTCCC
CTGCTAGGGCAATTTGCCCTAGCATCTGCTCCGTACATTAGGAACCGGCGGATGCTTCGCCCTCGATCAGGTTGCGG
TAGCGCATGACTAGGATCGGGCCAGCCTGCCCCGCCTCCTCCTTCAAATCGTACTCCGGCAGGTCATTTGACCCGAT
CAGCTTGCGCACGGTGAAACAGAACTTCTTGAACTCTCCGGCGCTGCCACTGCGTTCGTAGATCGTCTTGAACAACC
ATCTGGCTTCTGCCTTGCCTGCGGCGCGGCGTGCCAGGCGGTAGAGAAAACGGCCGATGCCGGGATCGATCAAAAAG
TAATCGGGGTGAACCGTCAGCACGTCCGGGTTCTTGCCTTCTGTGATCTCGCGGTACATCCAATCAGCTAGCTCGAT
CTCGATGTACTCCGGCCGCCCGGTTTCGCTCTTTACGATCTTGTAGCGGCTAATCAAGGCTTCACCCTCGGATACCG
TCACCAGGCGGCCGTTCTTGGCCTTCTTCGTACGCTGCATGGCAACGTGCGTGGTGTTTAACCGAATGCAGGTTTCT
ACCAGGTCGTCTTTCTGCTTTCCGCCATCGGCTCGCCGGCAGAACTTGAGTACGTCCGCAACGTGTGGACGGAACAC
GCGGCCGGGCTTGTCTCCCTTCCCTTCCCGGTATCGGTTCATGGATTCGGTTAGATGGGAAACCGCCATCAGTACCA
GGTCGTAATCCCACACACTGGCCATGCCGGCCGGCCCTGCGGAAACCTCTACGTGCCCGTCTGGAAGCTCGTAGCGG
ATCACCTCGCCAGCTCGTCGGTCACGCTTCGACAGACGGAAAACGGCCACGTCCATGATGCTGCGACTATCGCGGGT
GCCCACGTCATAGAGCATCGGAACGAAAAAATCTGGTTGCTCGTCGCCCTTGGGCGGCTTCCTAATCGACGGCGCAC
CGGCTGCCGGCGGTTGCCGGGATTCTTTGCGGATTCGATCAGCGGCCGCTTGCCACGATTCACCGGGGCGTGCTTCT
GCCTCGATGCGTTGCCGCTGGGCGGCCTGCGCGGCCTTCAACTTCTCCACCAGGTCATCACCCAGCGCCGCGCCGAT
TTGTACCGGGCCGGATGGTTTGCGACCGCTCACGCCGATTCCTCGGGCTTGGGGGTTCCAGTGCCATTGCAGGGCCG
GCAGGCAACCCAGCCGCTTACGCCTGGCCAACCGCCCGTTCCTCCACACATGGGGCATTCCACGGCGTCGGTGCCTG
GTTGTTCTTGATTTTCCATGCCGCCTCCTTTAGCCGCTAAAATTCATCTACTCATTTATTCATTTGCTCATTTACTC
TGGTAGCTGCGCGATGTATTCAGATAGCAGCTCGGTAATGGTCTTGCCTTGGCGTACCGCGTACATCTTCAGCTTGG
TGTGATCCTCCGCCGGCAACTGAAAGTTGACCCGCTTCATGGCTGGCGTGTCTGCCAGGCTGGCCAACGTTGCAGCC
TTGCTGCTGCGTGCGCTCGGACGGCCGGCACTTAGCGTGTTTGTGCTTTTGCTCATTTTCTCTTTACCTCATTAACT
CAAATGAGTTTTGATTTAATTTCAGCGGCCAGCGCCTGGACCTCGCGGGCAGCGTCGCCCTCGGGTTCTGATTCAAG
AACGGTTGTGCCGGCGGCGGCAGTGCCTGGGTAGCTCACGCGCTGCGTGATACGGGACTCAAGAATGGGCAGCTCGT
ACCCGGCCAGCGCCTCGGCAACCTCACCGCCGATGCGCGTGCCTTTGATCGCCCGCGACACGACAAAGGCCGCTTGT
AGCCTTCCATCCGTGACCTCAATGCGCTGCTTAACCAGCTCCACCAGGTCGGCGGTGGCCCATATGTCGTAAGGGCT
TGGCTGCACCGGAATCAGCACGAAGTCGGCTGCCTTGATCGCGGACACAGCCAAGTCCGCCGCCTGGGGCGCTCCGT
CGATCACTACGAAGTCGCGCCGGCCGATGGCCTTCACGTCGCGGTCAATCGTCGGGCGGTCGATGCCGACAACGGTT
AGCGGTTGATCTTCCCGCACGGCCGCCCAATCGCGGGCACTGCCCTGGGGATCGGAATCGACTAACAGAACATCGGC
CCCGGCGAGTTGCAGGGCGCGGGCTAGATGGGTTGCGATGGTCGTCTTGCCTGACCCGCCTTTCTGGTTAAGTACAG
CGATAACCTTCATGCGTTCCCCTTGCGTATTTGTTTATTTACTCATCGCATCATATACGCAGCGACCGCATGACGCA
AGCTGTTTTACTCAAATACACATCACCTTTTTAGACGGCGGCGCTCGGTTTCTTCAGCGGCCAAGCTGGCCGGCCAG
GCCGCCAGCTTGGCATCAGACAAACCGGCCAGGATTTCATGCAGCCGCACGGTTGAGACGTGCGCGGGCGGCTCGAA
CACGTACCCGGCCGCGATCATCTCCGCCTCGATCTCTTCGGTAATGAAAAACGGTTCGTCCTGGCCGTCCTGGTGCG
GTTTCATGCTTGTTCCTCTTGGCGTTCATTCTCGGCGGCCGCCAGGGCGTCGGCCTCGGTCAATGCGTCCTCACGGA
AGGCACCGCGCCGCCTGGCCTCGGTGGGCGTCACTTCCTCGCTGCGCTCAAGTGCGCGGTACAGGGTCGAGCGATGC
ACGCCAAGCAGTGCAGCCGCCTCTTTCACGGTGCGGCCTTCCTGGTCGATCAGCTCGCGGGCGTGCGCGATCTGTGC
CGGGGTGAGGGTAGGGCGGGGGCCAAACTTCACGCCTCGGGCCTTGGCGGCCTCGCGCCCGCTCCGGGTGCGGTCGA
TGATTAGGGAACGCTCGAACTCGGCAATGCCGGCGAACACGGTCAACACCATGCGGCCGGCCGGCGTGGTGGTGTCG
GCCCACGGCTCTGCCAGGCTACGCAGGCCCGCGCCGGCCTCCTGGATGCGCTCGGCAATGTCCAGTAGGTCGCGGGT
GCTGCGGGCCAGGCGGTCTAGCCTGGTCACTGTCACAACGTCGCCAGGGCGTAGGTGGTCAAGCATCCTGGCCAGCT
CCGGGCGGTCGCGCCTGGTGCCGGTGATCTTCTCGGAAAACAGCTTGGTGCAGCCGGCCGCGTGCAGTTCGGCCCGT
TGGTTGGTCAAGTCCTGGTCGTCGGTGCTGACGCGGGCATAGCCCAGCAGGCCAGCGGCGGCGCTCTTGTTCATGGC
GTAATGTCTCCGGTTCTAGTCGCAAGTATTCTACTTTATGCGACTAAAACACGCGACAAGAAAACGCCAGGAAAAGG
GCAGGGCGGCAGCCTGTCGCGTAACTTAGGACTTGTGCGACATGTCGTTTTCAGAAGACGGCTGCACTGAACGTCAG
AAGCCGACTGCACTATAGCAGCGGAGGGGTTGGATCAAAGTACTTTGATCCCGAGGGGAACCCTGTGGTTGGCATGC
ACATACAAATGGACGAACGGATAAACCTTTTCACGCCCTTTTAAATATCCGATTATTCTAATAAACGCTCTTTTCTC
TTAGGTTTACCCGCCAATATATCCTGTCAAACACTGATAGTTTAAAAGCTTTCCCGATCTAGTAACATAGATGACAC
CGCGCGCGATAATTTATCCTAGTTTGCGCGCTATATTTTGTTTTCTATCGCGTATTAAATGTATAATTGCGGGACTC
TAATCATAAAAACCCATCTCATAAATAACGTCATGCATTACATGTTAATTATTACATGCTTAACGTAATTCAACAGA
AATTATATGATAATCATCGCAAGACCGGCAACAGGATTCAATCTTAAGAAACTTTATTGCCAAATGTTTGAACGATC
GGGGAAATTCGAGCTACCACTTTGTACAAGAAAGCTGGGTTTAAGCCTTCTTGATAGCAGCAGCGAGTTCATCGTAA
GCAACTTCCCAAGCCCTAGACAATTCATCTGACCATTTATCACCAACAGCAGCTTTAATAGTTTTAAGCAAAGCTTC
TTTCACAACAACGAACTGAGGATCTGTAACAGCCTTTTGAGCATGAACTGAACCCAAAGCAGCATCAGCAACAACTG
TCCCAGAAGCTTTAAGCTGACCAGCTGAATCTCTCACCAGAGCAAAAAGCTTTTCTGCATGTCCAGTCAACTTAGGA
TTTGTTGGATCAACACCGTTAGCCAAAAATGAAAACAAATCCTTAGCAGCAGGAGCTTTTTCCAAAATTGATGTGTA
GAAAACCACAGAATATTGTGGAATATTTGCCTTGAAAGCTTCAAATGATGATGAAACAAGAGCATCTTGCTTTTCAG
TAAAAGCAACCATGGTGGCAGCCTGCTTTTTTGTACAAACTTGTCAGCGTGTCCTCTCCAAATGAAATGAACTTCCT
TATATAGAGGAAGGGTCTTGCGAAGGATAGTGGGATTGTGCGTCATCCCTTACGTCAGTGGAGATATCACATCAATC
CACTTGCTTTGAAGACGTGGTTGGAACGTCTTCTTTTTCCACGATGCTCCTCGTGGGTGGGGGTCCATCTTTGGGAC
CACTGTCGGCAGAGGCATCTTGAACGATAGCCTTTCCTTTATCGCAATGATGGCATTTGTAGGTGCCACCTTCCTTT
TCTACTGTCCTTTTGATGAAGTGACAGATAGCTGGGCAATGGAATCCGAGGAGGTTTCCCGATATTACCCTTTGTTG
AAAAGTCTCAATAGCCCTTTGGTCTTCTGAGACTGTATCTTTGATATTCTTGGAGTAGACGAGAGTGTCGTGCTCCA
CCATGTTCACATCAATCCACTTGCTTTGAAGACGTGGTTGGAACGTCTTCTTTTTCCACGATGCTCCTCGTGGGTGG
GGGTCCATCTTTGGGACCACTGTCGGCAGAGGCATCTTGAACGATAGCCTTTCCTTTATCGCAATGATGGCATTTGT
AGGTGCCACCTTCCTTTTCTACTGTCCTTTTGATGAAGTGACAGATAGCTGGGCAATGGAATCCGAGGAGGTTTCCC
GATATTACCCTTTGTTGAAAAGTCTCAATAGCCCTTTGGTCTTCTGAGACTGTATCTTTGATATTCTTGGAGTAGAC
GAGAGTGTCGTGCTCCACCATGTTGGCCAACTTTTCTATACAAAGTTG

In some embodiments, a vector of the present disclosure (e.g., a plasmid) is useful for delivering a heme-producing gene and a male-sterility gene to a plant cell (e.g., a tomato cell or an eggplant cell).

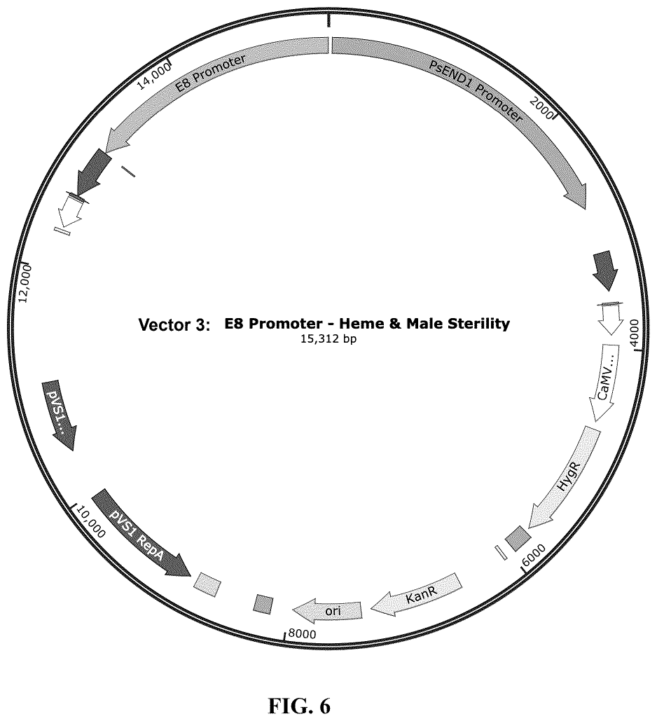

A non-limiting example of a vector comprising a codon-optimized heme-producing gene flanked by a plant promoter (e.g., E8 promoter) and a NOS terminator, a male-sterility gene (e.g., a barnase gene) flanked by a Pisum sativum Endothecium 1 (PSEND1) promoter and a NOS terminator, a hygromycin resistance gene under control of a CaMV 35S promoter and a CaMV polyA termination signal, and a left border (LB) T-DNA repeat and a right border (RB) T-DNA repeat is shown in FIG. 6 and provided as SEQ ID NO: 3:

(SEQ ID NO: 3)
AGCTTGCATGCCTGCAGGTCGACTCTAGAGGATCGACTTCAACCTTATTAGTGAATGGACAATAAAGGTTATAAGCT
CCTTTACTGTGAAAGCCCACCAGTAACATCACCTTGCTTATATCATTCAGCTTCTTTCTAGTAACATTTGGAACGTG
TTTATAACAGAAAAAAACCCAAAAACTCTGAAAAGACTCACACTTTTCTTATCTCCAGTCCACCTCTCAAAAGGAAC
AATTTCCTTCAGCTTCTTGGTTGGACACCTGTTGAGCACATATGCTGCAGTGGCAACAGTTTCTCCCCACAAAGTGT
TAGGAAGCTTCTTCTCCTTCAGCATGTTCCTTGTCATATCAAGCAAAGTTCGGTTTTCAACAAGACCATTATGTTGA
GGAGTATATGGATCAGTCACTTCATGCTCAATTCCATTCTCTTTACAGAACTTCTTGAACTCTGTAGAGTTATACTC
ACCTCCACCATCAGTTCTGAGAATCTTCAGAAGTCTGACCACTTTATTTCTCAGCCTTGATTATGAATTTCTTAAAT
TCAGCAAACACCTCGTGTTTGAATTTTATAAGGGATACCCATGTCATCCTTGTGAACTCATCCATAAATGACATAAA
GTATTATTCCCTCCTAGTGAAAGGTTTGTAATGGGCCACACACATAAGAATGCACTACTCCTAAAGCATGTTTTGCT
CTTTGAGCTACTTTTGATGAAAATGGCAGTCTTGGTTGCTTCCCTTTCATGCACACATTACATGACTTTTTTGGTTT
CTTAATTGTAGGAATTCCACGTACCAGTTTCTTTGAATTCAAATTCCCTAAGCTCCTAAAGTTCAAATGACCAAATC
TTTTGTTCCACAACTCACTTTCCTTCACAACACTTGTTGCGCTAAGGCATTCAGAGTCTGCAGTTTTAACATTCGCC
TTGAATGTTTTACTCCTTCCATGTTCTGACTCCATAATCAACTTCTGATAACAGTCATACAGCTTCAAAAGAATGTC
ATTCATGGTAACTGGAAATCCCTTTTCAATTAATTGACCTACACTCATCAGATTGCTCTTCATGCCAAGAACGTACC
AAGACGTTCTGAATTAATGCAGATTTTCTATTATTCATAATCACTCTAACATTCCCCATTCCTTTAGCATTTAGTTA
CTTATCATCAGCACATCTAATCTTGGTTTTCTTCCTAGAGTCAAAATCAACCAGCCATTTCTTATTTCCAGTATGAT
GGTTTGAACAACCAGTGTCCATATATCACCAGTCTTCTATAGACGCACTATCATAACTAGAAGCCATTAATAGCACA
TGTTCATCATGGTGCTCAGATCCTTAGAATGTTCAATTGCTACAACGATGTAATCAAACTGATGAGTAAGAGATCTA
AGTACCTTCTCAATGATACTTTCCTCATAAAGAGTTTCTCCATGCGACTTCATCTCATTTGTGATCAGAATCACTCT
AGAGATGTAGTCAGATAACTTCTCATTGTTCTTCATGCTTAGATTCTCATACTGCTCACGTAGAGACTGAAGTTTCA
CCTTCTACACTGATGCATCACTATCGTAGCACCACACCAGTCTGTCTCACACAACCTTTTCCGTCATTGAATCAACG
ATTTTCTTAAACACGTTCACATCCACACACTGATGGATGTAGAACAACGCATTCTGATCCTTCTTCCTCATATCACA
CTGAGCATTTCTTTGCGCATCCGTTGCATTTTCTAGAAGTGAAGCATAAACTTCGTTGATGAGATCAAGAACATCTT
GAGCACCAAATAACACACACATCTGAATCATCCAACGATTCCAGTTGTTGTCGTCGAACAATGGNAGCNTGGTGCAC
AGATTCACAACGATATATTATAANTTTTGTTTTATGAAATTTAAGAACAAATTTCCATTATTCTTAAAATGTTTACA
CACTGATGTAGACTGCAAAAGGAATAAAGATACAATTTGTTCACACCACTCACTTGCGTAAATATAAGTGAGAGTTA
ATGAGAAATACTAAAATACCCTCTAAAATTATGAATTAATTCTAACAATCTCTAATGTTAGTATAATCCATTAAACA
CTTTGATGGCAGGTATAACAAGGGTGTAAGTTAGTGTATACATATTAGGCTCTTATTATTTTTATATTATCTCTGCT
TTTCTTCTTCATGTTCTCACTAATATGATATTATCTCCCTTCCCTAAATTATTTATATTTATTAGAAAAAGAGTTTC
ATTTTTTAAAAATATATTACCGTAATTTTTCAAAAAATAAAATTTAAATATATTTTATAAAAAAATTATTTAATAAT
TTATTTACATTAATGCATAAATATAAATAAATACTGTCATTTAATATTTAACCTTTTAACAATAAATTATATTTATT
TAATTCAACTAATATAAGCTAAGTTATCTCATCCAACCAATTAAAAAGATCATTTGAAAATACCTTTTTATTTAGTT
TGTGGCGGTTTCAACTGTCAAAAAAAAGGAATTTTTACGACGATATAAATTTAAACCAGCAAAAAATTGAAGCAGTT
AAGCGAACCAACTCATGGTATGTGGATATATTTATCTTTGTCGTTTATATCGGATTCGAATCTCTATAATGATGAAA
AATTAATATCAAACTTTAAATAAGAACGTCATTTATAGAGCCATTTTGGGAAACACATATTTCATGTACACGTGATT
CGCAAATTTCCAATAACTCTATATATAGCCCTCCTCAGTTTCATGCATTTGCTCACAACATAACCTTCCTTGAATCT
GGAAAACGTCACATTGCTTCCGCATATCGGGTCAGCAACGGCTAAAATCCGCTTGAATATGTTCACACAAGCCGCTC
AAAACATGATTGACGCCGTATACGGAAGAACGCCGAAAAACCTTACTAAGGAATTTCAATAAGAAGAAAAATCCCGG
TTGGTTCAGCCGGGGTTTATTTTTCGCTAGATAAAAAGTACTATTTTTAAATTCTTTCTATTCCTTTCTTTCGTTGC
TGATACAATGAAAAGGAATCAGCTTCACATGATGAAAATGGGAGGTATTGCTTTGAAAAAACGATTATCGTGGATTT
CCGTTTGTTTACTGGTGCTTGTCTCCGCGGCGGGGATGCTGTTTTCAACAGCTGCCAAAACGGAAACATCTTCTCAC
AAGGCACACACAGAAGCACAGGTTATCAACACGTTTGACGGGGTTGCGGATTATCTTCAGACATATCATAAGCTACC
TGATAATTACATTACAAAATCAGAAGCACAAGCCCTCGGCTGGGTGGCATCAAAAGGGAACCTTGCAGACGTCGCTC
CGGGGAAAAGCATCGGCGGAGACATCTTCTCAAACAGGGAAGGCAAACTCCCGGGCAAAAGCGGACGAACATGGCGT
GAAGCGGATATTAACTATACATCAGGCTTCAGAAATTCAGACCGGATTCTTTACTCAAGCGACTGGCTGATTTACAA
AACAACGGACCATTATCAGACCTTTACAAAAATCAGATAACGAAAAAAACGGCTTCCCTGCGGAGGCCGTTTTTTTC
AGCTTTACATAAAGTGTGTAATAAATTTTTCTTCAAACTCTGATCGGTCAATTTCACTTTGCTAGCCACCACCACCA
CCACCACGTGTGAGATCGTTCAAACATTTGGCAATAAAGTTTCTTAAGATTGAATCCTGTTGCCGGTCTTGCGATGA
TTATCATATAATTTCTGTTGAATTACGTTAAGCATGTAATAATTAACATGTAATGCATGACGTTATTTATGAGATGG
GTTTTTATGATTAGAGTCCCGCAATTATACATTTAATACGCGATAGAAAACAAAATATAGCGCGCAAACTAGGATAA
ATTATCGCGCGCGGTGTCATCTATGTTACTAGATCCCGGGTACGCCAACATGGTGGAGCACGACACTCTCGTCTACT
CCAAGAATATCAAAGATACAGTCTCAGAAGACCAAAGGGCTATTGAGACTTTTCAACAAAGGGTAATATCGGGAAAC
CTCCTCGGATTCCATTGCCCAGCTATCTGTCACTTCATCAAAAGGACAGTAGAAAAGGAAGGTGGCACCTACAAATG
CCATCATTGCGATAAAGGAAAGGCTATCGTTCAAGATGCCTCTGCCGACAGTGGTCCCAAAGATGGACCCCCACCCA
CGAGGAGCATCGTGGAAAAAGAAGACGTTCCAACCACGTCTTCAAAGCAAGTGGATTGATGTGAACATGGTGGAGCA
CGACACTCTCGTCTACTCCAAGAATATCAAAGATACAGTCTCAGAAGACCAAAGGGCTATTGAGACTTTTCAACAAA
GGGTAATATCGGGAAACCTCCTCGGATTCCATTGCCCAGCTATCTGTCACTTCATCAAAAGGACAGTAGAAAAGGAA
GGTGGCACCTACAAATGCCATCATTGCGATAAAGGAAAGGCTATCGTTCAAGATGCCTCTGCCGACAGTGGTCCCAA
AGATGGACCCCCACCCACGAGGAGCATCGTGGAAAAAGAAGACGTTCCAACCACGTCTTCAAAGCAAGTGGATTGAT
GTGATATCTCCACTGACGTAAGGGATGACGCACAATCCCACTATCCTTCGCAAGACCCTTCCTCTATATAAGGAAGT
TCATTTCATTTGGAGAGGACACGCTGAAATCACCAGTCTCTCTCTACAAATCTATCTCTCTCGAGCTTTCGCAGATC
CGGGGGGCAATGAGATATGAAAAAGCCTGAACTCACCGCGACGTCTGTCGAGAAGTTTCTGATCGAAAAGTTCGACA
GCGTCTCCGACCTGATGCAGCTCTCGGAGGGCGAAGAATCTCGTGCTTTCAGCTTCGATGTAGGAGGGCGTGGATAT
GTCCTGCGGGTAAATAGCTGCGCCGATGGTTTCTACAAAGATCGTTATGTTTATCGGCACTTTGCATCGGCCGCGCT
CCCGATTCCGGAAGTGCTTGACATTGGGGAGTTTAGCGAGAGCCTGACCTATTGCATCTCCCGCCGTTCACAGGGTG
TCACGTTGCAAGACCTGCCTGAAACCGAACTGCCCGCTGTTCTACAACCGGTCGCGGAGGCTATGGATGCGATCGCT
GCGGCCGATCTTAGCCAGACGAGCGGGTTCGGCCCATTCGGACCGCAAGGAATCGGTCAATACACTACATGGCGTGA
TTTCATATGCGCGATTGCTGATCCCCATGTGTATCACTGGCAAACTGTGATGGACGACACCGTCAGTGCGTCCGTCG
CGCAGGCTCTCGATGAGCTGATGCTTTGGGCCGAGGACTGCCCCGAAGTCCGGCACCTCGTGCACGCGGATTTCGGC
TCCAACAATGTCCTGACGGACAATGGCCGCATAACAGCGGTCATTGACTGGAGCGAGGCGATGTTCGGGGATTCCCA
ATACGAGGTCGCCAACATCTTCTTCTGGAGGCCGTGGTTGGCTTGTATGGAGCAGCAGACGCGCTACTTCGAGCGGA
GGCATCCGGAGCTTGCAGGATCGCCACGACTCCGGGCGTATATGCTCCGCATTGGTCTTGACCAACTCTATCAGAGC
TTGGTTGACGGCAATTTCGATGATGCAGCTTGGGCGCAGGGTCGATGCGACGCAATCGTCCGATCCGGAGCCGGGAC
TGTCGGGCGTACACAAATCGCCCGCAGAAGCGCGGCCGTCTGGACCGATGGCTGTGTAGAAGTACTCGCCGATAGTG
GAAACCGACGCCCCAGCACTCGTCCGAGGGCAAAGAAATAGAGTAGATGCCGACCGGGATCTGTCGATCGACAAGCT
CGAGTTTCTCCATAATAATGTGTGAGTAGTTCCCAGATAAGGGAATTAGGGTTCCTATAGGGTTTCGCTCATGTGTT
GAGCATATAAGAAACCCTTAGTATGTATTTGTATTTGTAAAATACTTCTATCAATAAAATTTCTAATTCCTAAAACC
AAAATCCAGTACTAAAATCCAGATCCCCCGGTACCGAGCTCGAATTCAATTCGGCGTTAATTCAGTACATTAAAAAC
GTCCGCAATGTGTTATTAAGTTGTCTAAGCGTCAATTTGTTTACACCACAATATATCCTGCCACCAGCCAGCCAACA
GCTCCCCGACCGGCAGCTCGGCACAAAATCACCACTCGATACAGGCAGCCCATCAGTCCGGGACGGCGTCAGCGGGA
GAGCCGTTGTAAGGCGGCAGACTTTGCTCATGTTACCGATGCTATTCGGAAGAACGGCAACTAAGCTGCCGGGTTTG
AAACACGGATGATCTCGCGGAGGGTAGCATGTTGATTGTAACGATGACAGAGCGTTGCTGCCTGTGATCACCGCGGT
TTCAAAATCGGCTCCGTCGATACTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCTTCTTGGGGTATCTTTAAATACT
GTAGAAAAGAGGAAGGAAATAATAAATGGCTAAAATGAGAATATCACCGGAATTGAAAAAACTGATCGAAAAATACC
GCTGCGTAAAAGATACGGAAGGAATGTCTCCTGCTAAGGTATATAAGCTGGTGGGAGAAAATGAAAACCTATATTTA
AAAATGACGGACAGCCGGTATAAAGGGACCACCTATGATGTGGAACGGGAAAAGGACATGATGCTATGGCTGGAAGG
AAAGCTGCCTGTTCCAAAGGTCCTGCACTTTGAACGGCATGATGGCTGGAGCAATCTGCTCATGAGTGAGGCCGATG
GCGTCCTTTGCTCGGAAGAGTATGAAGATGAACAAAGCCCTGAAAAGATTATCGAGCTGTATGCGGAGTGCATCAGG
CTCTTTCACTCCATCGACATATCGGATTGTCCCTATACGAATAGCTTAGACAGCCGCTTAGCCGAATTGGATTACTT
ACTGAATAACGATCTGGCCGATGTGGATTGCGAAAACTGGGAAGAAGACACTCCATTTAAAGATCCGCGCGAGCTGT
ATGATTTTTTAAAGACGGAAAAGCCCGAAGAGGAACTTGTCTTTTCCCACGGCGACCTGGGAGACAGCAACATCTTT
GTGAAAGATGGCAAAGTAAGTGGCTTTATTGATCTTGGGAGAAGCGGCAGGGCGGACAAGTGGTATGACATTGCCTT
CTGCGTCCGGTCGATCAGGGAGGATATCGGGGAAGAACAGTATGTCGAGCTATTTTTTGACTTACTGGGGATCAAGC
CTGATTGGGAGAAAATAAAATATTATATTTTACTGGATGAATTGTTTTAGTACCTAGAATGCATGACCAAAATCCCT
TAACGTGAGTTTTCGTTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCTAGTGTAGCCGTAGTTAGGC
CACCACTTCAAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGGAAGCGGAAGAGCGCCTGATGCGGTATT
TTCTCCTTACGCATCTGTGCGGTATTTCACACCGCATATGGTGCACTCTCAGTACAATCTGCTCTGATGCCGCATAG
TTAAGCCAGTATACACTCCGCTATCGCTACGTGACTGGGTCATGGCTGCGCCCCGACACCCGCCAACACCCGCTGAC
GCGCCCTGACGGGCTTGTCTGCTCCCGGCATCCGCTTACAGACAAGCTGTGACCGTCTCCGGGAGCTGCATGTGTCA
GAGGTTTTCACCGTCATCACCGAAACGCGCGAGGCAGGGTGCCTTGATGTGGGCGCCGGCGGTCGAGTGGCGACGGC
GCGGCTTGTCCGCGCCCTGGTAGATTGCCTGGCCGTAGGCCAGCCATTTTTGAGCGGCCAGCGGCCGCGATAGGCCG
ACGCGAAGCGGCGGGGCGTAGGGAGCGCAGCGACCGAAGGGTAGGCGCTTTTTGCAGCTCTTCGGCTGTGCGCTGGC
CAGACAGTTATGCACAGGCCAGGCGGGTTTTAAGAGTTTTAATAAGTTTTAAAGAGTTTTAGGCGGAAAAATCGCCT
TTTTTCTCTTTTATATCAGTCACTTACATGTGTGACCGGTTCCCAATGTACGGCTTTGGGTTCCCAATGTACGGGTT
CCGGTTCCCAATGTACGGCTTTGGGTTCCCAATGTACGTGCTATCCACAGGAAAGAGACCTTTTCGACCTTTTTCCC
CTGCTAGGGCAATTTGCCCTAGCATCTGCTCCGTACATTAGGAACCGGCGGATGCTTCGCCCTCGATCAGGTTGCGG
TAGCGCATGACTAGGATCGGGCCAGCCTGCCCCGCCTCCTCCTTCAAATCGTACTCCGGCAGGTCATTTGACCCGAT
CAGCTTGCGCACGGTGAAACAGAACTTCTTGAACTCTCCGGCGCTGCCACTGCGTTCGTAGATCGTCTTGAACAACC
ATCTGGCTTCTGCCTTGCCTGCGGCGCGGCGTGCCAGGCGGTAGAGAAAACGGCCGATGCCGGGATCGATCAAAAAG
TAATCGGGGTGAACCGTCAGCACGTCCGGGTTCTTGCCTTCTGTGATCTCGCGGTACATCCAATCAGCTAGCTCGAT
CTCGATGTACTCCGGCCGCCCGGTTTCGCTCTTTACGATCTTGTAGCGGCTAATCAAGGCTTCACCCTCGGATACCG
TCACCAGGCGGCCGTTCTTGGCCTTCTTCGTACGCTGCATGGCAACGTGCGTGGTGTTTAACCGAATGCAGGTTTCT
ACCAGGTCGTCTTTCTGCTTTCCGCCATCGGCTCGCCGGCAGAACTTGAGTACGTCCGCAACGTGTGGACGGAACAC
GCGGCCGGGCTTGTCTCCCTTCCCTTCCCGGTATCGGTTCATGGATTCGGTTAGATGGGAAACCGCCATCAGTACCA
GGTCGTAATCCCACACACTGGCCATGCCGGCCGGCCCTGCGGAAACCTCTACGTGCCCGTCTGGAAGCTCGTAGCGG
ATCACCTCGCCAGCTCGTCGGTCACGCTTCGACAGACGGAAAACGGCCACGTCCATGATGCTGCGACTATCGCGGGT
GCCCACGTCATAGAGCATCGGAACGAAAAAATCTGGTTGCTCGTCGCCCTTGGGCGGCTTCCTAATCGACGGCGCAC
CGGCTGCCGGCGGTTGCCGGGATTCTTTGCGGATTCGATCAGCGGCCGCTTGCCACGATTCACCGGGGCGTGCTTCT
GCCTCGATGCGTTGCCGCTGGGCGGCCTGCGCGGCCTTCAACTTCTCCACCAGGTCATCACCCAGCGCCGCGCCGAT
TTGTACCGGGCCGGATGGTTTGCGACCGCTCACGCCGATTCCTCGGGCTTGGGGGTTCCAGTGCCATTGCAGGGCCG
GCAGGCAACCCAGCCGCTTACGCCTGGCCAACCGCCCGTTCCTCCACACATGGGGCATTCCACGGCGTCGGTGCCTG
GTTGTTCTTGATTTTCCATGCCGCCTCCTTTAGCCGCTAAAATTCATCTACTCATTTATTCATTTGCTCATTTACTC
TGGTAGCTGCGCGATGTATTCAGATAGCAGCTCGGTAATGGTCTTGCCTTGGCGTACCGCGTACATCTTCAGCTTGG
TGTGATCCTCCGCCGGCAACTGAAAGTTGACCCGCTTCATGGCTGGCGTGTCTGCCAGGCTGGCCAACGTTGCAGCC
TTGCTGCTGCGTGCGCTCGGACGGCCGGCACTTAGCGTGTTTGTGCTTTTGCTCATTTTCTCTTTACCTCATTAACT
CAAATGAGTTTTGATTTAATTTCAGCGGCCAGCGCCTGGACCTCGCGGGCAGCGTCGCCCTCGGGTTCTGATTCAAG
AACGGTTGTGCCGGCGGCGGCAGTGCCTGGGTAGCTCACGCGCTGCGTGATACGGGACTCAAGAATGGGCAGCTCGT
ACCCGGCCAGCGCCTCGGCAACCTCACCGCCGATGCGCGTGCCTTTGATCGCCCGCGACACGACAAAGGCCGCTTGT
AGCCTTCCATCCGTGACCTCAATGCGCTGCTTAACCAGCTCCACCAGGTCGGCGGTGGCCCATATGTCGTAAGGGCT
TGGCTGCACCGGAATCAGCACGAAGTCGGCTGCCTTGATCGCGGACACAGCCAAGTCCGCCGCCTGGGGCGCTCCGT
CGATCACTACGAAGTCGCGCCGGCCGATGGCCTTCACGTCGCGGTCAATCGTCGGGCGGTCGATGCCGACAACGGTT
AGCGGTTGATCTTCCCGCACGGCCGCCCAATCGCGGGCACTGCCCTGGGGATCGGAATCGACTAACAGAACATCGGC
CCCGGCGAGTTGCAGGGCGCGGGCTAGATGGGTTGCGATGGTCGTCTTGCCTGACCCGCCTTTCTGGTTAAGTACAG
CGATAACCTTCATGCGTTCCCCTTGCGTATTTGTTTATTTACTCATCGCATCATATACGCAGCGACCGCATGACGCA
AGCTGTTTTACTCAAATACACATCACCTTTTTAGACGGCGGCGCTCGGTTTCTTCAGCGGCCAAGCTGGCCGGCCAG
GCCGCCAGCTTGGCATCAGACAAACCGGCCAGGATTTCATGCAGCCGCACGGTTGAGACGTGCGCGGGCGGCTCGAA
CACGTACCCGGCCGCGATCATCTCCGCCTCGATCTCTTCGGTAATGAAAAACGGTTCGTCCTGGCCGTCCTGGTGCG
GTTTCATGCTTGTTCCTCTTGGCGTTCATTCTCGGCGGCCGCCAGGGCGTCGGCCTCGGTCAATGCGTCCTCACGGA
AGGCACCGCGCCGCCTGGCCTCGGTGGGCGTCACTTCCTCGCTGCGCTCAAGTGCGCGGTACAGGGTCGAGCGATGC
ACGCCAAGCAGTGCAGCCGCCTCTTTCACGGTGCGGCCTTCCTGGTCGATCAGCTCGCGGGCGTGCGCGATCTGTGC
CGGGGTGAGGGTAGGGCGGGGGCCAAACTTCACGCCTCGGGCCTTGGCGGCCTCGCGCCCGCTCCGGGTGCGGTCGA
TGATTAGGGAACGCTCGAACTCGGCAATGCCGGCGAACACGGTCAACACCATGCGGCCGGCCGGCGTGGTGGTGTCG
GCCCACGGCTCTGCCAGGCTACGCAGGCCCGCGCCGGCCTCCTGGATGCGCTCGGCAATGTCCAGTAGGTCGCGGGT
GCTGCGGGCCAGGCGGTCTAGCCTGGTCACTGTCACAACGTCGCCAGGGCGTAGGTGGTCAAGCATCCTGGCCAGCT
CCGGGCGGTCGCGCCTGGTGCCGGTGATCTTCTCGGAAAACAGCTTGGTGCAGCCGGCCGCGTGCAGTTCGGCCCGT
TGGTTGGTCAAGTCCTGGTCGTCGGTGCTGACGCGGGCATAGCCCAGCAGGCCAGCGGCGGCGCTCTTGTTCATGGC
GTAATGTCTCCGGTTCTAGTCGCAAGTATTCTACTTTATGCGACTAAAACACGCGACAAGAAAACGCCAGGAAAAGG
GCAGGGCGGCAGCCTGTCGCGTAACTTAGGACTTGTGCGACATGTCGTTTTCAGAAGACGGCTGCACTGAACGTCAG
AAGCCGACTGCACTATAGCAGCGGAGGGGTTGGATCAAAGTACTTTGATCCCGAGGGGAACCCTGTGGTTGGCATGC
ACATACAAATGGACGAACGGATAAACCTTTTCACGCCCTTTTAAATATCCGATTATTCTAATAAACGCTCTTTTCTC
TTAGGTTTACCCGCCAATATATCCTGTCAAACACTGATAGTTTAAAAGCTTTCCCGATCTAGTAACATAGATGACAC
CGCGCGCGATAATTTATCCTAGTTTGCGCGCTATATTTTGTTTTCTATCGCGTATTAAATGTATAATTGCGGGACTC
TAATCATAAAAACCCATCTCATAAATAACGTCATGCATTACATGTTAATTATTACATGCTTAACGTAATTCAACAGA
AATTATATGATAATCATCGCAAGACCGGCAACAGGATTCAATCTTAAGAAACTTTATTGCCAAATGTTTGAACGATC
GGGGAAATTCGAGCTACCACTTTGTACAAGAAAGCTGGGTTTAAGCCTTCTTGATAGCAGCAGCGAGTTCATCGTAA
GCAACTTCCCAAGCCCTAGACAATTCATCTGACCATTTATCACCAACAGCAGCTTTAATAGTTTTAAGCAAAGCTTC
TTTCACAACAACGAACTGAGGATCTGTAACAGCCTTTTGAGCATGAACTGAACCCAAAGCAGCATCAGCAACAACTG
TCCCAGAAGCTTTAAGCTGACCAGCTGAATCTCTCACCAGAGCAAAAAGCTTTTCTGCATGTCCAGTCAACTTAGGA
TTTGTTGGATCAACACCGTTAGCCAAAAATGAAAACAAATCCTTAGCAGCAGGAGCTTTTTCCAAAATTGATGTGTA
GAAAACCACAGAATATTGTGGAATATTTGCCTTGAAAGCTTCAAATGATGATGAAACAAGAGCATCTTGCTTTTCAG
TAAAAGCAACCATGGTGGCGGATCCCTTCTTTTGCACTGTGAATGATTAGAATAATTTCTAAAAATCTCAATATGAG
GATGCCATATTTATAATAGAATAAAATAAAATGTGAACAAAGAAAGAGATAAAGTAGTTCACTTTTTGAAATCTAAG
AGAGAAATGGGAACAAGAAAGAGACAAAAGTAGTTTCAAACAAACTTCTCTTCTAAGTTTAGTCCCTTTTTAAAATA
TGAAACCCAATACGTCTGATTAAGAATAGAAAAATATCAAATTTTCAATATAATTTATACTAATCGTTTTGAATTTT
TCATACTGATATAGTGTACGTTTCATCATAACAACCAAAACGTTGTTGTTTCACAACAATAATATAGTAGTAGTTAA
TTTATTATTTAGTAATAAGTGGTCCTAAAAATTAGATAAATATTACTATGATAATATAAAAATATTTGAGTCAGTCC
TAAAAAATTATTTAGTATTCATACATGAATCAAACTAATTAGTTAAGTGTCAACAATTGGACAAGTGGCATGGAGGT
TGTAAAAGAATGACATAAGCCAACTGCTATTTTTATCCAAAAAAAAGAAGACAACTTGACAACTACATTTCTTTTAT
TTTTATAAATTTACTAATATCTTCTATGCAAAATTATTCGGTGCCTTTCTAAACTTTAAGGTTTTTATTTGATGTAC
ACCTAAATTATATTTTATTTTAATCACTTCACTGAACTTGTTTATTCCTTCATCATATACACCTACTCCTATTATGA
CTACAAGTTGGCAAAAGTAATGATATGAATTTCTACTTAAATAAATAATAGTCACCTAGATAAATTAATTTAACAAA
AGATAAATATCAAACCTTCTCACCTAAAATTTTGAGCAAAACTTCTCACTAAAACTTGTGGACTAAACCCGAAAATC
TTCAGAAAATTAATATTTAGTACTGGAAAAGTCAGATTAAATGTCTGCACAAGACTTTCTATTGTTGGGAATAAACA
AATTAATATTGGATTAAAATAGTTGAAATATTTAGGTAAAATGCTACATGTCATTTATTCATTGGAAATTATTTCTT
AAAATTTAAAATTCATTATTTAAAAGTTATTTTTGAAAAAGGGCCGATTTCTGAAATTCCTTCTAAGATAGGGTCTT
TCTAGACGTAAAGTTGATCTATTAAATTTTAAATTTATCTTAAATTCTTACAAAGTAAGTATTAATCTTTGTTTCCT
TTACTATTCATTTACATTTTGTCCTATATTTCGTTTAAAATATGTCATATATTAAAAAAAATTAAAAATTTTACTTT
CTTTTTTTACGTTATAGCTATATGACGTGACAAAAAATCAACTTTCACATGCGCCTAGTAGACTTCAAGTTAAAAGG
GGATAATGGATACTTTGCCTATCTTTTACCATATATTTTAAAATCCTTAATTATTAAGTTTTCCAATATCTCTCACC
ATTCATTTTCTCCTATCATATATTTTAGGAGTCCTTAATAATTAAGTTTACTAATAAACTTTATTATATATTATAGG
ACTCCTCAATTATTAGTTCTCTTTATGTCTCTCATCGTACATTTTCCTCTTGTCTTATTTGTTAGGACACTTGAAAT
TTTCAAAATATATTTTGCTTTTAATATATGAAGTTGTGTTTGATTGTAGTTTTTGTAAATATATTTAATTTTTTGAA
TTTTTATTTTCTAAAAGAAACATAAAATTTAAAAGATTTAAAAGTATCATTAAACTATTAGAAATAATATATCTATG
TTGTTAAAAATGATGGTTCTTAATTAACTGTTTTATTATAAAATATCAGATAATTCGTTTTATTTACGCAAAAGTTA
AGTGAAGTAACGAAATTATAAATCCCATAGAATATTGTGTATATACTTGGCACATGATGATTGTAACATCCTTAATT
ATTATTAATTCATCGAACCTATTATTTCTTCATTGTCTATGTACATTTATCCTTAATAATTCCACTTCAGGATTTAT
TAGTTCTTTGGTTATTGGTTTAAGTTTATTTTACAACCAAGTGAATTGAATTTGTCCTCCATTAATATTTATTGGAT
TAAAAAATAAATAAATTTGCTCTTATTTGTAGAAAGATTTAGACTTTTAAAATATTACGTTTTCTGACTCTTTTCTT
ATCAAAATTGGACTCTCTCACTTCCACAAAACTTAATTACATGAACAATATCATTAGGGAAAGCTT

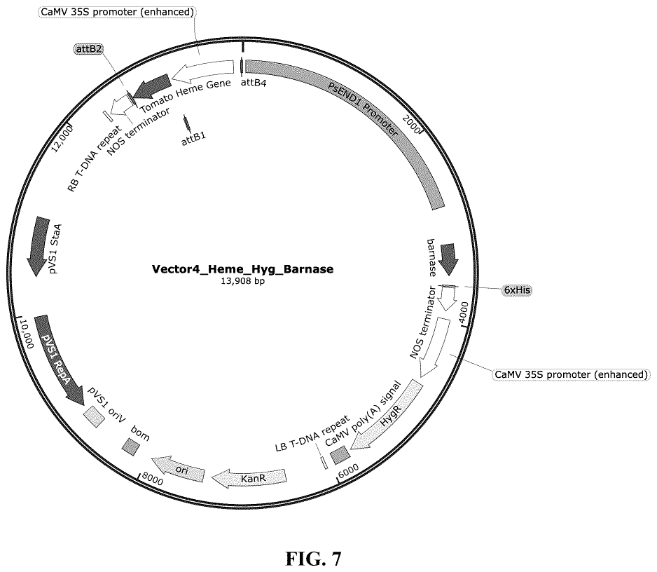

A non-limiting example of a vector comprising a codon-optimized heme-producing gene flanked by a CaMV 35S promoter and a NOS terminator, a male-sterility gene (e.g., a barnase gene) flanked by a Pisum sativum Endothecium 1 (PSEND1) promoter and a NOS terminator, a hygromycin resistance gene under control of a CaMV 35S promoter and a CaMV polyA termination signal, and a left border (LB) T-DNA repeat and a right border (RB) T-DNA repeat is shown in FIG. 7 and provided as SEQ ID NO: 4:

(SEQ ID NO: 4)
AGCTTGCATGCCTGCAGGTCGACTCTAGAGGATCGACTTCAACCTTATTAGTGAATGGACAATAAAGGTTATAAGCT
CCTTTACTGTGAAAGCCCACCAGTAACATCACCTTGCTTATATCATTCAGCTTCTTTCTAGTAACATTTGGAACGTG
TTTATAACAGAAAAAAACCCAAAAACTCTGAAAAGACTCACACTTTTCTTATCTCCAGTCCACCTCTCAAAAGGAAC
AATTTCCTTCAGCTTCTTGGTTGGACACCTGTTGAGCACATATGCTGCAGTGGCAACAGTTTCTCCCCACAAAGTGT
TAGGAAGCTTCTTCTCCTTCAGCATGTTCCTTGTCATATCAAGCAAAGTTCGGTTTTCAACAAGACCATTATGTTGA
GGAGTATATGGATCAGTCACTTCATGCTCAATTCCATTCTCTTTACAGAACTTCTTGAACTCTGTAGAGTTATACTC
ACCTCCACCATCAGTTCTGAGAATCTTCAGAAGTCTGACCACTTTATTTCTCAGCCTTGATTATGAATTTCTTAAAT
TCAGCAAACACCTCGTGTTTGAATTTTATAAGGGATACCCATGTCATCCTTGTGAACTCATCCATAAATGACATAAA
GTATTATTCCCTCCTAGTGAAAGGTTTGTAATGGGCCACACACATAAGAATGCACTACTCCTAAAGCATGTTTTGCT
CTTTGAGCTACTTTTGATGAAAATGGCAGTCTTGGTTGCTTCCCTTTCATGCACACATTACATGACTTTTTTGGTTT
CTTAATTGTAGGAATTCCACGTACCAGTTTCTTTGAATTCAAATTCCCTAAGCTCCTAAAGTTCAAATGACCAAATC
TTTTGTTCCACAACTCACTTTCCTTCACAACACTTGTTGCGCTAAGGCATTCAGAGTCTGCAGTTTTAACATTCGCC
TTGAATGTTTTACTCCTTCCATGTTCTGACTCCATAATCAACTTCTGATAACAGTCATACAGCTTCAAAAGAATGTC
ATTCATGGTAACTGGAAATCCCTTTTCAATTAATTGACCTACACTCATCAGATTGCTCTTCATGCCAAGAACGTACC
AAGACGTTCTGAATTAATGCAGATTTTCTATTATTCATAATCACTCTAACATTCCCCATTCCTTTAGCATTTAGTTA
CTTATCATCAGCACATCTAATCTTGGTTTTCTTCCTAGAGTCAAAATCAACCAGCCATTTCTTATTTCCAGTATGAT
GGTTTGAACAACCAGTGTCCATATATCACCAGTCTTCTATAGACGCACTATCATAACTAGAAGCCATTAATAGCACA
TGTTCATCATGGTGCTCAGATCCTTAGAATGTTCAATTGCTACAACGATGTAATCAAACTGATGAGTAAGAGATCTA
AGTACCTTCTCAATGATACTTTCCTCATAAAGAGTTTCTCCATGCGACTTCATCTCATTTGTGATCAGAATCACTCT
AGAGATGTAGTCAGATAACTTCTCATTGTTCTTCATGCTTAGATTCTCATACTGCTCACGTAGAGACTGAAGTTTCA
CCTTCTACACTGATGCATCACTATCGTAGCACCACACCAGTCTGTCTCACACAACCTTTTCCGTCATTGAATCAACG
ATTTTCTTAAACACGTTCACATCCACACACTGATGGATGTAGAACAACGCATTCTGATCCTTCTTCCTCATATCACA
CTGAGCATTTCTTTGCGCATCCGTTGCATTTTCTAGAAGTGAAGCATAAACTTCGTTGATGAGATCAAGAACATCTT
GAGCACCAAATAACACACACATCTGAATCATCCAACGATTCCAGTTGTTGTCGTCGAACAATGGNAGCNTGGTGCAC
AGATTCACAACGATATATTATAANTTTTGTTTTATGAAATTTAAGAACAAATTTCCATTATTCTTAAAATGTTTACA
CACTGATGTAGACTGCAAAAGGAATAAAGATACAATTTGTTCACACCACTCACTTGCGTAAATATAAGTGAGAGTTA
ATGAGAAATACTAAAATACCCTCTAAAATTATGAATTAATTCTAACAATCTCTAATGTTAGTATAATCCATTAAACA
CTTTGATGGCAGGTATAACAAGGGTGTAAGTTAGTGTATACATATTAGGCTCTTATTATTTTTATATTATCTCTGCT
TTTCTTCTTCATGTTCTCACTAATATGATATTATCTCCCTTCCCTAAATTATTTATATTTATTAGAAAAAGAGTTTC
ATTTTTTAAAAATATATTACCGTAATTTTTCAAAAAATAAAATTTAAATATATTTTATAAAAAAATTATTTAATAAT
TTATTTACATTAATGCATAAATATAAATAAATACTGTCATTTAATATTTAACCTTTTAACAATAAATTATATTTATT
TAATTCAACTAATATAAGCTAAGTTATCTCATCCAACCAATTAAAAAGATCATTTGAAAATACCTTTTTATTTAGTT
TGTGGCGGTTTCAACTGTCAAAAAAAAGGAATTTTTACGACGATATAAATTTAAACCAGCAAAAAATTGAAGCAGTT
AAGCGAACCAACTCATGGTATGTGGATATATTTATCTTTGTCGTTTATATCGGATTCGAATCTCTATAATGATGAAA
AATTAATATCAAACTTTAAATAAGAACGTCATTTATAGAGCCATTTTGGGAAACACATATTTCATGTACACGTGATT
CGCAAATTTCCAATAACTCTATATATAGCCCTCCTCAGTTTCATGCATTTGCTCACAACATAACCTTCCTTGAATCT
GGAAAACGTCACATTGCTTCCGCATATCGGGTCAGCAACGGCTAAAATCCGCTTGAATATGTTCACACAAGCCGCTC
AAAACATGATTGACGCCGTATACGGAAGAACGCCGAAAAACCTTACTAAGGAATTTCAATAAGAAGAAAAATCCCGG
TTGGTTCAGCCGGGGTTTATTTTTCGCTAGATAAAAAGTACTATTTTTAAATTCTTTCTATTCCTTTCTTTCGTTGC
TGATACAATGAAAAGGAATCAGCTTCACATGATGAAAATGGGAGGTATTGCTTTGAAAAAACGATTATCGTGGATTT
CCGTTTGTTTACTGGTGCTTGTCTCCGCGGCGGGGATGCTGTTTTCAACAGCTGCCAAAACGGAAACATCTTCTCAC
AAGGCACACACAGAAGCACAGGTTATCAACACGTTTGACGGGGTTGCGGATTATCTTCAGACATATCATAAGCTACC
TGATAATTACATTACAAAATCAGAAGCACAAGCCCTCGGCTGGGTGGCATCAAAAGGGAACCTTGCAGACGTCGCTC
CGGGGAAAAGCATCGGCGGAGACATCTTCTCAAACAGGGAAGGCAAACTCCCGGGCAAAAGCGGACGAACATGGCGT
GAAGCGGATATTAACTATACATCAGGCTTCAGAAATTCAGACCGGATTCTTTACTCAAGCGACTGGCTGATTTACAA
AACAACGGACCATTATCAGACCTTTACAAAAATCAGATAACGAAAAAAACGGCTTCCCTGCGGAGGCCGTTTTTTTC
AGCTTTACATAAAGTGTGTAATAAATTTTTCTTCAAACTCTGATCGGTCAATTTCACTTTGCTAGCCACCACCACCA
CCACCACGTGTGAGATCGTTCAAACATTTGGCAATAAAGTTTCTTAAGATTGAATCCTGTTGCCGGTCTTGCGATGA
TTATCATATAATTTCTGTTGAATTACGTTAAGCATGTAATAATTAACATGTAATGCATGACGTTATTTATGAGATGG
GTTTTTATGATTAGAGTCCCGCAATTATACATTTAATACGCGATAGAAAACAAAATATAGCGCGCAAACTAGGATAA
ATTATCGCGCGCGGTGTCATCTATGTTACTAGATCCCGGGTACGCCAACATGGTGGAGCACGACACTCTCGTCTACT
CCAAGAATATCAAAGATACAGTCTCAGAAGACCAAAGGGCTATTGAGACTTTTCAACAAAGGGTAATATCGGGAAAC
CTCCTCGGATTCCATTGCCCAGCTATCTGTCACTTCATCAAAAGGACAGTAGAAAAGGAAGGTGGCACCTACAAATG
CCATCATTGCGATAAAGGAAAGGCTATCGTTCAAGATGCCTCTGCCGACAGTGGTCCCAAAGATGGACCCCCACCCA
CGAGGAGCATCGTGGAAAAAGAAGACGTTCCAACCACGTCTTCAAAGCAAGTGGATTGATGTGAACATGGTGGAGCA
CGACACTCTCGTCTACTCCAAGAATATCAAAGATACAGTCTCAGAAGACCAAAGGGCTATTGAGACTTTTCAACAAA
GGGTAATATCGGGAAACCTCCTCGGATTCCATTGCCCAGCTATCTGTCACTTCATCAAAAGGACAGTAGAAAAGGAA
GGTGGCACCTACAAATGCCATCATTGCGATAAAGGAAAGGCTATCGTTCAAGATGCCTCTGCCGACAGTGGTCCCAA
AGATGGACCCCCACCCACGAGGAGCATCGTGGAAAAAGAAGACGTTCCAACCACGTCTTCAAAGCAAGTGGATTGAT
GTGATATCTCCACTGACGTAAGGGATGACGCACAATCCCACTATCCTTCGCAAGACCCTTCCTCTATATAAGGAAGT
TCATTTCATTTGGAGAGGACACGCTGAAATCACCAGTCTCTCTCTACAAATCTATCTCTCTCGAGCTTTCGCAGATC
CGGGGGGCAATGAGATATGAAAAAGCCTGAACTCACCGCGACGTCTGTCGAGAAGTTTCTGATCGAAAAGTTCGACA
GCGTCTCCGACCTGATGCAGCTCTCGGAGGGCGAAGAATCTCGTGCTTTCAGCTTCGATGTAGGAGGGCGTGGATAT
GTCCTGCGGGTAAATAGCTGCGCCGATGGTTTCTACAAAGATCGTTATGTTTATCGGCACTTTGCATCGGCCGCGCT
CCCGATTCCGGAAGTGCTTGACATTGGGGAGTTTAGCGAGAGCCTGACCTATTGCATCTCCCGCCGTTCACAGGGTG
TCACGTTGCAAGACCTGCCTGAAACCGAACTGCCCGCTGTTCTACAACCGGTCGCGGAGGCTATGGATGCGATCGCT
GCGGCCGATCTTAGCCAGACGAGCGGGTTCGGCCCATTCGGACCGCAAGGAATCGGTCAATACACTACATGGCGTGA
TTTCATATGCGCGATTGCTGATCCCCATGTGTATCACTGGCAAACTGTGATGGACGACACCGTCAGTGCGTCCGTCG
CGCAGGCTCTCGATGAGCTGATGCTTTGGGCCGAGGACTGCCCCGAAGTCCGGCACCTCGTGCACGCGGATTTCGGC
TCCAACAATGTCCTGACGGACAATGGCCGCATAACAGCGGTCATTGACTGGAGCGAGGCGATGTTCGGGGATTCCCA
ATACGAGGTCGCCAACATCTTCTTCTGGAGGCCGTGGTTGGCTTGTATGGAGCAGCAGACGCGCTACTTCGAGCGGA
GGCATCCGGAGCTTGCAGGATCGCCACGACTCCGGGCGTATATGCTCCGCATTGGTCTTGACCAACTCTATCAGAGC
TTGGTTGACGGCAATTTCGATGATGCAGCTTGGGCGCAGGGTCGATGCGACGCAATCGTCCGATCCGGAGCCGGGAC
TGTCGGGCGTACACAAATCGCCCGCAGAAGCGCGGCCGTCTGGACCGATGGCTGTGTAGAAGTACTCGCCGATAGTG
GAAACCGACGCCCCAGCACTCGTCCGAGGGCAAAGAAATAGAGTAGATGCCGACCGGGATCTGTCGATCGACAAGCT
CGAGTTTCTCCATAATAATGTGTGAGTAGTTCCCAGATAAGGGAATTAGGGTTCCTATAGGGTTTCGCTCATGTGTT
GAGCATATAAGAAACCCTTAGTATGTATTTGTATTTGTAAAATACTTCTATCAATAAAATTTCTAATTCCTAAAACC
AAAATCCAGTACTAAAATCCAGATCCCCCGGTACCGAGCTCGAATTCAATTCGGCGTTAATTCAGTACATTAAAAAC
GTCCGCAATGTGTTATTAAGTTGTCTAAGCGTCAATTTGTTTACACCACAATATATCCTGCCACCAGCCAGCCAACA
GCTCCCCGACCGGCAGCTCGGCACAAAATCACCACTCGATACAGGCAGCCCATCAGTCCGGGACGGCGTCAGCGGGA
GAGCCGTTGTAAGGCGGCAGACTTTGCTCATGTTACCGATGCTATTCGGAAGAACGGCAACTAAGCTGCCGGGTTTG
AAACACGGATGATCTCGCGGAGGGTAGCATGTTGATTGTAACGATGACAGAGCGTTGCTGCCTGTGATCACCGCGGT
TTCAAAATCGGCTCCGTCGATACTATGTTATACGCCAACTTTGAAAACAACTTTGAAAAAGCTGTTTTCTGGTATTT
AAGGTTTTAGAATGCAAGGAACAGTGAATTGGAGTTCGTCTTGTTATAATTAGCTTCTTGGGGTATCTTTAAATACT
GTAGAAAAGAGGAAGGAAATAATAAATGGCTAAAATGAGAATATCACCGGAATTGAAAAAACTGATCGAAAAATACC
GCTGCGTAAAAGATACGGAAGGAATGTCTCCTGCTAAGGTATATAAGCTGGTGGGAGAAAATGAAAACCTATATTTA
AAAATGACGGACAGCCGGTATAAAGGGACCACCTATGATGTGGAACGGGAAAAGGACATGATGCTATGGCTGGAAGG
AAAGCTGCCTGTTCCAAAGGTCCTGCACTTTGAACGGCATGATGGCTGGAGCAATCTGCTCATGAGTGAGGCCGATG
GCGTCCTTTGCTCGGAAGAGTATGAAGATGAACAAAGCCCTGAAAAGATTATCGAGCTGTATGCGGAGTGCATCAGG
CTCTTTCACTCCATCGACATATCGGATTGTCCCTATACGAATAGCTTAGACAGCCGCTTAGCCGAATTGGATTACTT
ACTGAATAACGATCTGGCCGATGTGGATTGCGAAAACTGGGAAGAAGACACTCCATTTAAAGATCCGCGCGAGCTGT
ATGATTTTTTAAAGACGGAAAAGCCCGAAGAGGAACTTGTCTTTTCCCACGGCGACCTGGGAGACAGCAACATCTTT
GTGAAAGATGGCAAAGTAAGTGGCTTTATTGATCTTGGGAGAAGCGGCAGGGCGGACAAGTGGTATGACATTGCCTT
CTGCGTCCGGTCGATCAGGGAGGATATCGGGGAAGAACAGTATGTCGAGCTATTTTTTGACTTACTGGGGATCAAGC
CTGATTGGGAGAAAATAAAATATTATATTTTACTGGATGAATTGTTTTAGTACCTAGAATGCATGACCAAAATCCCT
TAACGTGAGTTTTCGTTCCACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTCCTTCTAGTGTAGCCGTAGTTAGGC
CACCACTTCAAGAACTCTGTAGCACCGCCTACATACCTCGCTCTGCTAATCCTGTTACCAGTGGCTGCTGCCAGTGG
CGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGGAAGCGGAAGAGCGCCTGATGCGGTATT
TTCTCCTTACGCATCTGTGCGGTATTTCACACCGCATATGGTGCACTCTCAGTACAATCTGCTCTGATGCCGCATAG
TTAAGCCAGTATACACTCCGCTATCGCTACGTGACTGGGTCATGGCTGCGCCCCGACACCCGCCAACACCCGCTGAC
GCGCCCTGACGGGCTTGTCTGCTCCCGGCATCCGCTTACAGACAAGCTGTGACCGTCTCCGGGAGCTGCATGTGTCA
GAGGTTTTCACCGTCATCACCGAAACGCGCGAGGCAGGGTGCCTTGATGTGGGCGCCGGCGGTCGAGTGGCGACGGC
GCGGCTTGTCCGCGCCCTGGTAGATTGCCTGGCCGTAGGCCAGCCATTTTTGAGCGGCCAGCGGCCGCGATAGGCCG
ACGCGAAGCGGCGGGGCGTAGGGAGCGCAGCGACCGAAGGGTAGGCGCTTTTTGCAGCTCTTCGGCTGTGCGCTGGC
CAGACAGTTATGCACAGGCCAGGCGGGTTTTAAGAGTTTTAATAAGTTTTAAAGAGTTTTAGGCGGAAAAATCGCCT
TTTTTCTCTTTTATATCAGTCACTTACATGTGTGACCGGTTCCCAATGTACGGCTTTGGGTTCCCAATGTACGGGTT
CCGGTTCCCAATGTACGGCTTTGGGTTCCCAATGTACGTGCTATCCACAGGAAAGAGACCTTTTCGACCTTTTTCCC
CTGCTAGGGCAATTTGCCCTAGCATCTGCTCCGTACATTAGGAACCGGCGGATGCTTCGCCCTCGATCAGGTTGCGG
TAGCGCATGACTAGGATCGGGCCAGCCTGCCCCGCCTCCTCCTTCAAATCGTACTCCGGCAGGTCATTTGACCCGAT
CAGCTTGCGCACGGTGAAACAGAACTTCTTGAACTCTCCGGCGCTGCCACTGCGTTCGTAGATCGTCTTGAACAACC
ATCTGGCTTCTGCCTTGCCTGCGGCGCGGCGTGCCAGGCGGTAGAGAAAACGGCCGATGCCGGGATCGATCAAAAAG
TAATCGGGGTGAACCGTCAGCACGTCCGGGTTCTTGCCTTCTGTGATCTCGCGGTACATCCAATCAGCTAGCTCGAT
CTCGATGTACTCCGGCCGCCCGGTTTCGCTCTTTACGATCTTGTAGCGGCTAATCAAGGCTTCACCCTCGGATACCG
TCACCAGGCGGCCGTTCTTGGCCTTCTTCGTACGCTGCATGGCAACGTGCGTGGTGTTTAACCGAATGCAGGTTTCT
ACCAGGTCGTCTTTCTGCTTTCCGCCATCGGCTCGCCGGCAGAACTTGAGTACGTCCGCAACGTGTGGACGGAACAC
GCGGCCGGGCTTGTCTCCCTTCCCTTCCCGGTATCGGTTCATGGATTCGGTTAGATGGGAAACCGCCATCAGTACCA
GGTCGTAATCCCACACACTGGCCATGCCGGCCGGCCCTGCGGAAACCTCTACGTGCCCGTCTGGAAGCTCGTAGCGG
ATCACCTCGCCAGCTCGTCGGTCACGCTTCGACAGACGGAAAACGGCCACGTCCATGATGCTGCGACTATCGCGGGT
GCCCACGTCATAGAGCATCGGAACGAAAAAATCTGGTTGCTCGTCGCCCTTGGGCGGCTTCCTAATCGACGGCGCAC
CGGCTGCCGGCGGTTGCCGGGATTCTTTGCGGATTCGATCAGCGGCCGCTTGCCACGATTCACCGGGGCGTGCTTCT
GCCTCGATGCGTTGCCGCTGGGCGGCCTGCGCGGCCTTCAACTTCTCCACCAGGTCATCACCCAGCGCCGCGCCGAT
TTGTACCGGGCCGGATGGTTTGCGACCGCTCACGCCGATTCCTCGGGCTTGGGGGTTCCAGTGCCATTGCAGGGCCG
GCAGGCAACCCAGCCGCTTACGCCTGGCCAACCGCCCGTTCCTCCACACATGGGGCATTCCACGGCGTCGGTGCCTG
GTTGTTCTTGATTTTCCATGCCGCCTCCTTTAGCCGCTAAAATTCATCTACTCATTTATTCATTTGCTCATTTACTC
TGGTAGCTGCGCGATGTATTCAGATAGCAGCTCGGTAATGGTCTTGCCTTGGCGTACCGCGTACATCTTCAGCTTGG
TGTGATCCTCCGCCGGCAACTGAAAGTTGACCCGCTTCATGGCTGGCGTGTCTGCCAGGCTGGCCAACGTTGCAGCC
TTGCTGCTGCGTGCGCTCGGACGGCCGGCACTTAGCGTGTTTGTGCTTTTGCTCATTTTCTCTTTACCTCATTAACT
CAAATGAGTTTTGATTTAATTTCAGCGGCCAGCGCCTGGACCTCGCGGGCAGCGTCGCCCTCGGGTTCTGATTCAAG
AACGGTTGTGCCGGCGGCGGCAGTGCCTGGGTAGCTCACGCGCTGCGTGATACGGGACTCAAGAATGGGCAGCTCGT
ACCCGGCCAGCGCCTCGGCAACCTCACCGCCGATGCGCGTGCCTTTGATCGCCCGCGACACGACAAAGGCCGCTTGT
AGCCTTCCATCCGTGACCTCAATGCGCTGCTTAACCAGCTCCACCAGGTCGGCGGTGGCCCATATGTCGTAAGGGCT
TGGCTGCACCGGAATCAGCACGAAGTCGGCTGCCTTGATCGCGGACACAGCCAAGTCCGCCGCCTGGGGCGCTCCGT
CGATCACTACGAAGTCGCGCCGGCCGATGGCCTTCACGTCGCGGTCAATCGTCGGGCGGTCGATGCCGACAACGGTT
AGCGGTTGATCTTCCCGCACGGCCGCCCAATCGCGGGCACTGCCCTGGGGATCGGAATCGACTAACAGAACATCGGC
CCCGGCGAGTTGCAGGGCGCGGGCTAGATGGGTTGCGATGGTCGTCTTGCCTGACCCGCCTTTCTGGTTAAGTACAG
CGATAACCTTCATGCGTTCCCCTTGCGTATTTGTTTATTTACTCATCGCATCATATACGCAGCGACCGCATGACGCA
AGCTGTTTTACTCAAATACACATCACCTTTTTAGACGGCGGCGCTCGGTTTCTTCAGCGGCCAAGCTGGCCGGCCAG
GCCGCCAGCTTGGCATCAGACAAACCGGCCAGGATTTCATGCAGCCGCACGGTTGAGACGTGCGCGGGCGGCTCGAA
CACGTACCCGGCCGCGATCATCTCCGCCTCGATCTCTTCGGTAATGAAAAACGGTTCGTCCTGGCCGTCCTGGTGCG
GTTTCATGCTTGTTCCTCTTGGCGTTCATTCTCGGCGGCCGCCAGGGCGTCGGCCTCGGTCAATGCGTCCTCACGGA
AGGCACCGCGCCGCCTGGCCTCGGTGGGCGTCACTTCCTCGCTGCGCTCAAGTGCGCGGTACAGGGTCGAGCGATGC
ACGCCAAGCAGTGCAGCCGCCTCTTTCACGGTGCGGCCTTCCTGGTCGATCAGCTCGCGGGCGTGCGCGATCTGTGC
CGGGGTGAGGGTAGGGCGGGGGCCAAACTTCACGCCTCGGGCCTTGGCGGCCTCGCGCCCGCTCCGGGTGCGGTCGA
TGATTAGGGAACGCTCGAACTCGGCAATGCCGGCGAACACGGTCAACACCATGCGGCCGGCCGGCGTGGTGGTGTCG
GCCCACGGCTCTGCCAGGCTACGCAGGCCCGCGCCGGCCTCCTGGATGCGCTCGGCAATGTCCAGTAGGTCGCGGGT
GCTGCGGGCCAGGCGGTCTAGCCTGGTCACTGTCACAACGTCGCCAGGGCGTAGGTGGTCAAGCATCCTGGCCAGCT
CCGGGCGGTCGCGCCTGGTGCCGGTGATCTTCTCGGAAAACAGCTTGGTGCAGCCGGCCGCGTGCAGTTCGGCCCGT
TGGTTGGTCAAGTCCTGGTCGTCGGTGCTGACGCGGGCATAGCCCAGCAGGCCAGCGGCGGCGCTCTTGTTCATGGC
GTAATGTCTCCGGTTCTAGTCGCAAGTATTCTACTTTATGCGACTAAAACACGCGACAAGAAAACGCCAGGAAAAGG
GCAGGGCGGCAGCCTGTCGCGTAACTTAGGACTTGTGCGACATGTCGTTTTCAGAAGACGGCTGCACTGAACGTCAG
AAGCCGACTGCACTATAGCAGCGGAGGGGTTGGATCAAAGTACTTTGATCCCGAGGGGAACCCTGTGGTTGGCATGC
ACATACAAATGGACGAACGGATAAACCTTTTCACGCCCTTTTAAATATCCGATTATTCTAATAAACGCTCTTTTCTC
TTAGGTTTACCCGCCAATATATCCTGTCAAACACTGATAGTTTAAAAGCTTTCCCGATCTAGTAACATAGATGACAC
CGCGCGCGATAATTTATCCTAGTTTGCGCGCTATATTTTGTTTTCTATCGCGTATTAAATGTATAATTGCGGGACTC
TAATCATAAAAACCCATCTCATAAATAACGTCATGCATTACATGTTAATTATTACATGCTTAACGTAATTCAACAGA
AATTATATGATAATCATCGCAAGACCGGCAACAGGATTCAATCTTAAGAAACTTTATTGCCAAATGTTTGAACGATC
GGGGAAATTCGAGCTACCACTTTGTACAAGAAAGCTGGGTTTAAGCCTTCTTGATAGCAGCAGCGAGTTCATCGTAA
GCAACTTCCCAAGCCCTAGACAATTCATCTGACCATTTATCACCAACAGCAGCTTTAATAGTTTTAAGCAAAGCTTC
TTTCACAACAACGAACTGAGGATCTGTAACAGCCTTTTGAGCATGAACTGAACCCAAAGCAGCATCAGCAACAACTG
TCCCAGAAGCTTTAAGCTGACCAGCTGAATCTCTCACCAGAGCAAAAAGCTTTTCTGCATGTCCAGTCAACTTAGGA
TTTGTTGGATCAACACCGTTAGCCAAAAATGAAAACAAATCCTTAGCAGCAGGAGCTTTTTCCAAAATTGATGTGTA
GAAAACCACAGAATATTGTGGAATATTTGCCTTGAAAGCTTCAAATGATGATGAAACAAGAGCATCTTGCTTTTCAG
TAAAAGCAACCATGGTGGCAGCCTGCTTTTTTGTACAAACTTGTCAGCGTGTCCTCTCCAAATGAAATGAACTTCCT
TATATAGAGGAAGGGTCTTGCGAAGGATAGTGGGATTGTGCGTCATCCCTTACGTCAGTGGAGATATCACATCAATC
CACTTGCTTTGAAGACGTGGTTGGAACGTCTTCTTTTTCCACGATGCTCCTCGTGGGTGGGGGTCCATCTTTGGGAC
CACTGTCGGCAGAGGCATCTTGAACGATAGCCTTTCCTTTATCGCAATGATGGCATTTGTAGGTGCCACCTTCCTTT
TCTACTGTCCTTTTGATGAAGTGACAGATAGCTGGGCAATGGAATCCGAGGAGGTTTCCCGATATTACCCTTTGTTG
AAAAGTCTCAATAGCCCTTTGGTCTTCTGAGACTGTATCTTTGATATTCTTGGAGTAGACGAGAGTGTCGTGCTCCA
CCATGTTCACATCAATCCACTTGCTTTGAAGACGTGGTTGGAACGTCTTCTTTTTCCACGATGCTCCTCGTGGGTGG
GGGTCCATCTTTGGGACCACTGTCGGCAGAGGCATCTTGAACGATAGCCTTTCCTTTATCGCAATGATGGCATTTGT
AGGTGCCACCTTCCTTTTCTACTGTCCTTTTGATGAAGTGACAGATAGCTGGGCAATGGAATCCGAGGAGGTTTCCC
GATATTACCCTTTGTTGAAAAGTCTCAATAGCCCTTTGGTCTTCTGAGACTGTATCTTTGATATTCTTGGAGTAGAC
GAGAGTGTCGTGCTCCACCATGTTGGCCAACTTTTCTATACAAAGTTG

### Genes

Aspects of the present disclosure relate to the expression of one or more genes (coding sequences) into a plant cell (e.g., a tomato plant cell or an eggplant plant cell).

In some embodiments, a gene of the present disclosure is a heme-producing gene. In some embodiments, a heme-producing gene is derived from Glycine max (soybean). In some embodiments, a heme-producing gene derived from Glycine max is a leghemoglobin gene. Leghemoglobin (also known as leghaemoglobin or legoglobin) is an oxygen-carrying phytoglobin found in the nitrogen-fixing root nodules of leguminous plants. It is produced by these plants in response to the roots being colonized by nitrogen-fixing bacteria, termed rhizobia, as part of the symbiotic interaction between plant and bacterium. Leghemoglobin has close chemical and structural similarities to hemoglobin, and, like hemoglobin, is red in color. Leghemoglobins are monomeric proteins with a mass of around 16 kDa, and are structurally similar to myoglobin. In some embodiments, leghemoglobin is expressed in a plant (e.g., tomato plant or eggplant plant) to mimic the color, taste, and/or texture of meat.

In some embodiments, a heme-producing gene is a functional homolog of leghemoglobin. The term “functional homolog,” as used herein, includes genes or proteins that share a common ancestor and perform the same or similar functions, even if they have different underlying structures. Non-limiting examples of functional homologs of leghemoglobin include myoglobin and hemoglobin.

In some embodiments, a gene of the present disclosure is codon-optimized. In some embodiments, a gene of the present disclosure is codon-optimized for expression in a tomato or eggplant. The term “codon-optimized,” as used herein, includes altering the DNA sequence of a gene without changing the amino acid sequence of the protein encoded by the gene. This is done to improve the efficiency of translation and improve protein yield in a specific host organism (e.g., a tomato plant or eggplant plant).

A non-limiting example of a codon-optimized leghemoglobin gene derived from Glycine max is provided as SEQ ID NO: 5:

(SEQ ID NO: 5)
ATGGTTGCTTTTACTGAAAAGCAAGATGCTCTTGTTTCATCATCATTTGAAGCTTTCAAGGCAAATATTCCACAATA
TTCTGTGGTTTTCTACACATCAATTTTGGAAAAAGCTCCTGCTGCTAAGGATTTGTTTTCATTTTTGGCTAACGGTG
TTGATCCAACAAATCCTAAGTTGACTGGACATGCAGAAAAGCTTTTTGCTCTGGTGAGAGATTCAGCTGGTCAGCTT
AAAGCTTCTGGGACAGTTGTTGCTGATGCTGCTTTGGGTTCAGTTCATGCTCAAAAGGCTGTTACAGATCCTCAGTT
CGTTGTTGTGAAAGAAGCTTTGCTTAAAACTATTAAAGCTGCTGTTGGTGATAAATGGTCAGATGAATTGTCTAGGG
CTTGGGAAGTTGCTTACGATGAACTCGCTGCTGCTATCAAGAAGGCTTAA

In some embodiments, a gene of the present disclosure is a male-sterility gene. The term “male-sterility gene,” as used herein, includes a gene that improves seed purity. In some embodiments, a male-sterility gene is derived from Bacillus amyloliquefaciens. In some embodiments, a male-sterility gene derived from Bacillus amyloliquefaciens is a barnase gene. A barnase gene (a portmanteau of “BActerial” “RiboNucleASE”) is a bacterial protein that consists of about 110 amino acids and has ribonuclease activity. In some embodiments, a barnase gene is codon-optimized.

A non-limiting example of a barnase gene derived from Bacillus amyloliquefaciens is provided as SEQ ID NO: 6:

(SEQ ID NO: 6)
GCACAGGTTATCAACACGTTTGACGGGGTTGCGGATTATCTTCAGACATATCATAAGCTACCTGATAATTACATTAC
AAAATCAGAAGCACAAGCCCTCGGCTGGGTGGCATCAAAAGGGAACCTTGCAGACGTCGCTCCGGGGAAAAGCATCG
GCGGAGACATCTTCTCAAACAGGGAAGGCAAACTCCCGGGCAAAAGCGGACGAACATGGCGTGAAGCGGATATTAAC
TATACATCAGGCTTCAGAAATTCAGACCGGATTCTTTACTCAAGCGACTGGCTGATTTACAAAACAACGGACCATTA
TCAGACCTTTACAAAAATCAGATAA

In some embodiments, a gene of the present disclosure is a selectable marker. The term “selectable marker,” as used herein, includes a gene that is introduced into a cell or organism to identify which cells or organisms have been genetically transformed or transfected (i.e., with a genetic construct or set of genetic constructs). In some embodiments, a selectable marker is a positive selectable marker that confers selective advantage to the cell or organism. In some embodiments, a positive selectable marker is an antibiotic resistance gene. Non-limiting examples of positive selectable markers include a hygromycin resistance gene, an nptII gene, a kanamycin resistance gene, a chloramphenicol resistance gene, and an ampicillin resistance gene. In some embodiments, a selectable marker is a negative selectable marker that eliminates or inhibits growth of the cell or organism upon selection. A non-limiting example of a negative selective marker includes the thymidine kinase gene. In some embodiments, a selectable marker of the present disclosure is an antibiotic resistance gene. In some embodiments, a gene of the present disclosure is an antibiotic resistance gene. The term “antibiotic resistance gene,” as used herein, includes genes that confer resistance to an antibiotic, often enabling the cell expressing the gene to modify the antibiotic, alter its target, or pump it out of the cell. Antibiotic resistance genes are often used in research to select for cells that express a genetic construct containing the antibiotic resistance gene. In some embodiments, a selectable marker of the present disclosure is a hygromycin resistance gene. In some embodiments, an antibiotic resistance gene is a hygromycin resistance gene. The term “hygromycin resistance gene,” also known as the “hph gene,” as used herein, includes a DNA sequence that encodes an enzyme called hygromycin phosphotransferase (HPT). HPT modifies the antibiotic hygromycin B, rendering it inactive and thus conferring resistance to the organism (e.g. plant) carrying the gene.

A non-limiting example of a hygromycin resistance gene is provided as SEQ ID NO: 7:

(SEQ ID NO: 7)
ATGAAAAAGCCTGAACTCACCGCGACGTCTGTCGAGAAGTTTCTGATCGAAAAGTTCGACAGCGTCTCCGACCTGAT
GCAGCTCTCGGAGGGCGAAGAATCTCGTGCTTTCAGCTTCGATGTAGGAGGGCGTGGATATGTCCTGCGGGTAAATA
GCTGCGCCGATGGTTTCTACAAAGATCGTTATGTTTATCGGCACTTTGCATCGGCCGCGCTCCCGATTCCGGAAGTG
CTTGACATTGGGGAGTTTAGCGAGAGCCTGACCTATTGCATCTCCCGCCGTTCACAGGGTGTCACGTTGCAAGACCT
GCCTGAAACCGAACTGCCCGCTGTTCTACAACCGGTCGCGGAGGCTATGGATGCGATCGCTGCGGCCGATCTTAGCC
AGACGAGCGGGTTCGGCCCATTCGGACCGCAAGGAATCGGTCAATACACTACATGGCGTGATTTCATATGCGCGATT
GCTGATCCCCATGTGTATCACTGGCAAACTGTGATGGACGACACCGTCAGTGCGTCCGTCGCGCAGGCTCTCGATGA
GCTGATGCTTTGGGCCGAGGACTGCCCCGAAGTCCGGCACCTCGTGCACGCGGATTTCGGCTCCAACAATGTCCTGA
CGGACAATGGCCGCATAACAGCGGTCATTGACTGGAGCGAGGCGATGTTCGGGGATTCCCAATACGAGGTCGCCAAC
ATCTTCTTCTGGAGGCCGTGGTTGGCTTGTATGGAGCAGCAGACGCGCTACTTCGAGCGGAGGCATCCGGAGCTTGC
AGGATCGCCACGACTCCGGGCGTATATGCTCCGCATTGGTCTTGACCAACTCTATCAGAGCTTGGTTGACGGCAATT
TCGATGATGCAGCTTGGGCGCAGGGTCGATGCGACGCAATCGTCCGATCCGGAGCCGGGACTGTCGGGCGTACACAA
ATCGCCCGCAGAAGCGCGGCCGTCTGGACCGATGGCTGTGTAGAAGTACTCGCCGATAGTGGAAACCGACGCCCCAG
CACTCGTCCGAGGGCAAAGAAATAG

In some embodiments, a gene of the present disclosure is a reporter gene such as β-glucuronidase (GUS) reporter gene. The term “reporter gene,” as used herein, includes a gene whose expression can be measured and is used to study other genes, understand gene regulation, or screen for successful transformations.

A non-limiting example of a GUS reporter gene is provided as SEQ ID NO: 8:

(SEQ ID NO: 8)
ATGGTAGATCTGAGGGTAAATTTCTAGTTTTTCTCCTTCATTTTCTTGGTTAGGACCCTTTTCTCTTTTTATTTTTT
TGAGCTTTGATCTTTCTTTAAACTGATCTATTTTTTAATTGATTGGTTATGGTGTAAATATTACATAGCTTTAACTG
ATAATCTGATTACTTTATTTCGTGTGTCTATGATGATGATGATAGTTACAGAACCGACGACTCGTCCGTCCTGTAGA
AACCCCAACCCGTGAAATCAAAAAACTCGACGGCCTGTGGGCATTCAGTCTGGATCGCGAAAACTGTGGAATTGATC
AGCGTTGGTGGGAAAGCGCGTTACAAGAAAGCCGGGCAATTGCTGTGCCAGGCAGTTTTAACGATCAGTTCGCCGAT
GCAGATATTCGTAATTATGCGGGCAACGTCTGGTATCAGCGCGAAGTCTTTATACCGAAAGGTTGGGCAGGCCAGCG
TATCGTGCTGCGTTTCGATGCGGTCACTCATTACGGCAAAGTGTGGGTCAATAATCAGGAAGTGATGGAGCATCAGG
GCGGCTATACGCCATTTGAAGCCGATGTCACGCCGTATGTTATTGCCGGGAAAAGTGTACGTATCACCGTTTGTGTG
AACAACGAACTGAACTGGCAGACTATCCCGCCGGGAATGGTGATTACCGACGAAAACGGCAAGAAAAAGCAGTCTTA
CTTCCATGATTTCTTTAACTATGCCGGAATCCATCGCAGCGTAATGCTCTACACCACGCCGAACACCTGGGTGGACG
ATATCACCGTGGTGACGCATGTCGCGCAAGACTGTAACCACGCGTCTGTTGACTGGCAGGTGGTGGCCAATGGTGAT
GTCAGCGTTGAACTGCGTGATGCGGATCAACAGGTGGTTGCAACTGGACAAGGCACTAGCGGGACTTTGCAAGTGGT
GAATCCGCACCTCTGGCAACCGGGTGAAGGTTATCTCTATGAACTCGAAGTCACAGCCAAAAGCCAGACAGAGTCTG
ATATCTACCCGCTTCGCGTCGGCATCCGGTCAGTGGCAGTGAAGGGCCAACAGTTCCTGATTAACCACAAACCGTTC
TACTTTACTGGCTTTGGTCGTCATGAAGATGCGGACTTACGTGGCAAAGGATTCGATAACGTGCTGATGGTGCACGA
CCACGCATTAATGGACTGGATTGGGGCCAACTCCTACCGTACCTCGCATTACCCTTACGCTGAAGAGATGCTCGACT
GGGCAGATGAACATGGCATCGTGGTGATTGATGAAACTGCTGCTGTCGGCTTTCAGCTGTCTTTAGGCATTGGTTTC
GAAGCGGGCAACAAGCCGAAAGAACTGTACAGCGAAGAGGCAGTCAACGGGGAAACTCAGCAAGCGCACTTACAGGC
GATTAAAGAGCTGATAGCGCGTGACAAAAACCACCCAAGCGTGGTGATGTGGAGTATTGCCAACGAACCGGATACCC
GTCCGCAAGGTGCACGGGAATATTTCGCGCCACTGGCGGAAGCAACGCGTAAACTCGACCCGACGCGTCCGATCACC
TGCGTCAATGTAATGTTCTGCGACGCTCACACCGATACCATCAGCGATCTCTTTGATGTGCTGTGCCTGAACCGTTA
TTACGGATGGTATGTCCAAAGCGGCGATTTGGAAACGGCAGAGAAGGTACTGGAAAAAGAACTTCTGGCCTGGCAGG
AGAAACTGCATCAGCCGATTATCATCACCGAATACGGCGTGGATACGTTAGCCGGGCTGCACTCAATGTACACCGAC
ATGTGGAGTGAAGAGTATCAGTGTGCATGGCTGGATATGTATCACCGCGTCTTTGATCGCGTCAGCGCCGTCGTCGG
TGAACAGGTATGGAATTTCGCCGATTTTGCGACCTCGCAAGGCATATTGCGCGTTGGCGGTAACAAGAAAGGGATCT
TCACTCGCGACCGCAAACCGAAGTCGGCGGCTTTTCTGCTGCAAAAACGCTGGACTGGCATGAACTTCGGTGAAAAA
CCGCAGCAGGGAGGCAAACAA

### Promoters

Aspects of the present disclosure relate to the use of a promoter to drive expression of a gene described elsewhere herein. The term “promoter,” as used herein includes a region of DNA located upstream of a gene where proteins, such as ribonucleic acid (RNA) polymerase and transcription factors, bind to initiate transcription of the gene. This process results in the production of RNA molecules, such as messenger RNA (mRNA), which are essential for gene expression. Promoters are specific sequences that serve as landing pads for the transcription machinery, playing an important role in regulating gene activity. In some embodiments, a promoter (e.g., a first promoter and/or a second promoter) is a eukaryotic promoter. Non-limiting examples of eukaryotic promoters include E8, E8S, PSEND1, TDH3, PGK1, PKC1, PDC1, TEF1, TEF2, RPL18B. SSA1, TDH2, PYK1, TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, a promoter (e.g., a first promoter and/or a second promoter) is a prokaryotic promoter (e.g., bacteriophage or bacterial promoter). Non-limiting examples of bacteriophage promoters include Pls1con, T3, T7, SP6, and PL. Non-limiting examples of bacterial promoters include PmgrB, Ptrc2, PCI857, Pbad, Plac/ara, Plac/fnr, Ptac, Ptet, Pcmt, and Pm. In some embodiments, any promoter known in the art and suitable for a selected host cell (e.g., tomato plant cell or eggplant plant cell) can be used.

In some embodiments, a promoter is operably linked to a gene. The term “operably linked.” as used herein, includes a functional relationship between nucleic acids, indicating that they are placed in a way that allows one nucleic acid sequence to control the expression of another.

In some embodiments, a genetic construct or set of genetic constructs of the present disclosure comprises one or more promoters. In some embodiments, a genetic construct or set of genetic constructs of the present disclosure comprises a first promoter and a second promoter. In some embodiments, the first promoter and the second promoter are different. In some embodiments, the first promoter and the second promoter are the same. In some embodiments, one promoter drives the expression of one or more genes. The first and second promoter may be the same type of promoter or may be the same, single promoter that effects the expression of both coding sequences, in an embodiment of any one of the compositions or methods provided herein.

In some embodiments, a promoter (e.g., a first promoter and/or a second promoter) is a constitutive promoter. The term “constitutive promoter,” includes an unregulated promoter that allows continuous transcription of a gene. Non-limiting examples of a constitutive promoter include CaMV 35S, TDH3, PGK1, PKC1, PDC1, TEF1, TEF2, RPL18B, SSA1, TDH2, PYK1, TPI1, HXT3, HXT7, ACT1, ADH1, ADH2, ENO2, CMV, and SOD1. In some embodiments, a constitutive promoter is a Cauliflower Mosaic Virus (CaMV 35S). Other constitutive promoters known to one of ordinary skill in the art are also contemplated in this application.

A non-limiting examples of a Cauliflower Mosaic Virus (CaMV 35S) promoter is provided as SEQ ID NO: 9:

(SEQ ID NO: 9)
TGAGACTTTTCAACAAAGGGTAATATCGGGAAACCTCCTCGGATTCCATTGCCCAGCTATCTGTCACTTCATCAAAA
GGACAGTAGAAAAGGAAGGTGGCACCTACAAATGCCATCATTGCGATAAAGGAAAGGCTATCGTTCAAGATGCCTCT
GCCGACAGTGGTCCCAAAGATGGACCCCCACCCACGAGGAGCATCGTGGAAAAAGAAGACGTTCCAACCACGTCTTC
AAAGCAAGTGGATTGATGTGAACATGGTGGAGCACGACACTCTCGTCTACTCCAAGAATATCAAAGATACAGTCTCA
GAAGACCAAAGGGCTATTGAGACTTTTCAACAAAGGGTAATATCGGGAAACCTCCTCGGATTCCATTGCCCAGCTAT
CTGTCACTTCATCAAAAGGACAGTAGAAAAGGAAGGTGGCACCTACAAATGCCATCATTGCGATAAAGGAAAGGCTA
TCGTTCAAGATGCCTCTGCCGACAGTGGTCCCAAAGATGGACCCCCACCCACGAGGAGCATCGTGGAAAAAGAAGAC
GTTCCAACCACGTCTTCAAAGCAAGTGGATTGATGTGATATCTCCACTGACGTAAGGGATGACGCACAATCCCACTA
TCCTTCGCAAGACCCTTCCTCTATATAAGGAAGTTCATTTCATTTGGAGAGGACACGCTGA

In some embodiments, a promoter is a tissue-specific promoter. The term “tissue-specific promoter,” as used herein, includes a DNA sequence that regulates gene expression in a particular tissue or cell type, ensuring that genes are activated only in the appropriate locations within an organism. In some embodiments, a tissue-specific promoter is a plant-specific promoter, a plant promoter, or a fruit-specific promoter. In some embodiments, a plant promoter is an anther-specific promoter. The term “anther-specific,” as used herein, includes a regulatory element used in plant genetic engineering to control gene expression specifically in the anthers. Non-limiting examples of plant promoters include an E8 promoter, Pisum sativum Endothecium 1 (PSEND1) promoter, and a TA29 promoter.

A non-limiting examples of an E8 promoter is provided as SEQ ID NO: 10:

(SEQ ID NO: 10)
AAGCTTTCCCTAATGATATTGTTCATGTAATTAAGTTTTGTGGAAGTGAGAGAGTCCAATTTTGATAAGAAAAGAGT
CAGAAAACGTAATATTTTAAAAGTCTAAATCTTTCTACAAATAAGAGCAAATTTATTTATTTTTTAATCCAATAAAT
ATTAATGGAGGACAAATTCAATTCACTTGGTTGTAAAATAAACTTAAACCAATAACCAAAGAACTAATAAATCCTGA
AGTGGAATTATTAAGGATAAATGTACATAGACAATGAAGAAATAATAGGTTCGATGAATTAATAATAATTAAGGATG
TTACAATCATCATGTGCCAAGTATATACACAATATTCTATGGGATTTATAATTTCGTTACTTCACTTAACTTTTGCG
TAAATAAAACGAATTATCTGATATTTTATAATAAAACAGTTAATTAAGAACCATCATTTTTAACAACATAGATATAT
TATTTCTAATAGTTTAATGATACTTTTAAATCTTTTAAATTTTATGTTTCTTTTAGAAAATAAAAATTCAAAAAATT
AAATATATTTACAAAAACTACAATCAAACACAACTTCATATATTAAAAGCAAAATATATTTTGAAAATTTCAAGTGT
CCTAACAAATAAGACAAGAGGAAAATGTACGATGAGAGACATAAAGAGAACTAATAATTGAGGAGTCCTATAATATA
TAATAAAGTTTATTAGTAAACTTAATTATTAAGGACTCCTAAAATATATGATAGGAGAAAATGAATGGTGAGAGATA
TTGGAAAACTTAATAATTAAGGATTTTAAAATATATGGTAAAAGATAGGCAAAGTATCCATTATCCCCTTTTAACTT
GAAGTCTACTAGGCGCATGTGAAAGTTGATTTTTTGTCACGTCATATAGCTATAACGTAAAAAAAGAAAGTAAAATT
TTTAATTTTTTTTAATATATGACATATTTTAAACGAAATATAGGACAAAATGTAAATGAATAGTAAAGGAAACAAAG
ATTAATACTTACTTTGTAAGAATTTAAGATAAATTTAAAATTTAATAGATCAACTTTACGTCTAGAAAGACCCTATC
TTAGAAGGAATTTCAGAAATCGGCCCTTTTTCAAAAATAACTTTTAAATAATGAATTTTAAATTTTAAGAAATAATT
TCCAATGAATAAATGACATGTAGCATTTTACCTAAATATTTCAACTATTTTAATCCAATATTAATTTGTTTATTCCC
AACAATAGAAAGTCTTGTGCAGACATTTAATCTGACTTTTCCAGTACTAAATATTAATTTTCTGAAGATTTTCGGGT
TTAGTCCACAAGTTTTAGTGAGAAGTTTTGCTCAAAATTTTAGGTGAGAAGGTTTGATATTTATCTTTTGTTAAATT
AATTTATCTAGGTGACTATTATTTATTTAAGTAGAAATTCATATCATTACTTTTGCCAACTTGTAGTCATAATAGGA
GTAGGTGTATATGATGAAGGAATAAACAAGTTCAGTGAAGTGATTAAAATAAAATATAATTTAGGTGTACATCAAAT
AAAAACCTTAAAGTTTAGAAAGGCACCGAATAATTTTGCATAGAAGATATTAGTAAATTTATAAAAATAAAAGAAAT
GTAGTTGTCAAGTTGTCTTCTTTTTTTTGGATAAAAATAGCAGTTGGCTTATGTCATTCTTTTACAACCTCCATGCC
ACTTGTCCAATTGTTGACACTTAACTAATTAGTTTGATTCATGTATGAATACTAAATAATTTTTTAGGACTGACTCA
AATATTTTTATATTATCATAGTAATATTTATCTAATTTTTAGGACCACTTATTACTAAATAATAAATTAACTACTAC
TATATTATTGTTGTGAAACAACAACGTTTTGGTTGTTATGATGAAACGTACACTATATCAGTATGAAAAATTCAAAA
CGATTAGTATAAATTATATTGAAAATTTGATATTTTTCTATTCTTAATCAGACGTATTGGGTTTCATATTTTAAAAA
GGGACTAAACTTAGAAGAGAAGTTTGTTTGAAACTACTTTTGTCTCTTTCTTGTTCCCATTTCTCTCTTAGATTTCA
AAAAGTGAACTACTTTATCTCTTTCTTTGTTCACATTTTATTTTATTCTATTATAAATATGGCATCCTCATATTGAG
ATTTTTAGAAATTATTCTAATCATTCACAGTGCAAAAGAAGGGATCC

A non-limiting examples of a PSEND1 promoter is provided as SEQ ID NO: 11:

(SEQ ID NO: 11)
GACTTCAACCTTATTAGTGAATGGACAATAAAGGTTATAAGCTCCTTTACTGTGAAAGCCCACCAGTAACATCACCT
TGCTTATATCATTCAGCTTCTTTCTAGTAACATTTGGAACGTGTTTATAACAGAAAAAAACCCAAAAACTCTGAAAA
GACTCACACTTTTCTTATCTCCAGTCCACCTCTCAAAAGGAACAATTTCCTTCAGCTTCTTGGTTGGACACCTGTTG
AGCACATATGCTGCAGTGGCAACAGTTTCTCCCCACAAAGTGTTAGGAAGCTTCTTCTCCTTCAGCATGTTCCTTGT
CATATCAAGCAAAGTTCGGTTTTCAACAAGACCATTATGTTGAGGAGTATATGGATCAGTCACTTCATGCTCAATTC
CATTCTCTTTACAGAACTTCTTGAACTCTGTAGAGTTATACTCACCTCCACCATCAGTTCTGAGAATCTTCAGAAGT
CTGACCACTTTATTTCTCAGCCTTGATTATGAATTTCTTAAATTCAGCAAACACCTCGTGTTTGAATTTTATAAGGG
ATACCCATGTCATCCTTGTGAACTCATCCATAAATGACATAAAGTATTATTCCCTCCTAGTGAAAGGTTTGTAATGG
GCCACACACATAAGAATGCACTACTCCTAAAGCATGTTTTGCTCTTTGAGCTACTTTTGATGAAAATGGCAGTCTTG
GTTGCTTCCCTTTCATGCACACATTACATGACTTTTTTGGTTTCTTAATTGTAGGAATTCCACGTACCAGTTTCTTT
GAATTCAAATTCCCTAAGCTCCTAAAGTTCAAATGACCAAATCTTTTGTTCCACAACTCACTTTCCTTCACAACACT
TGTTGCGCTAAGGCATTCAGAGTCTGCAGTTTTAACATTCGCCTTGAATGTTTTACTCCTTCCATGTTCTGACTCCA
TAATCAACTTCTGATAACAGTCATACAGCTTCAAAAGAATGTCATTCATGGTAACTGGAAATCCCTTTTCAATTAAT
TGACCTACACTCATCAGATTGCTCTTCATGCCAAGAACGTACCAAGACGTTCTGAATTAATGCAGATTTTCTATTAT
TCATAATCACTCTAACATTCCCCATTCCTTTAGCATTTAGTTACTTATCATCAGCACATCTAATCTTGGTTTTCTTC
CTAGAGTCAAAATCAACCAGCCATTTCTTATTTCCAGTATGATGGTTTGAACAACCAGTGTCCATATATCACCAGTC
TTCTATAGACGCACTATCATAACTAGAAGCCATTAATAGCACATGTTCATCATGGTGCTCAGATCCTTAGAATGTTC
AATTGCTACAACGATGTAATCAAACTGATGAGTAAGAGATCTAAGTACCTTCTCAATGATACTTTCCTCATAAAGAG
TTTCTCCATGCGACTTCATCTCATTTGTGATCAGAATCACTCTAGAGATGTAGTCAGATAACTTCTCATTGTTCTTC
ATGCTTAGATTCTCATACTGCTCACGTAGAGACTGAAGTTTCACCTTCTACACTGATGCATCACTATCGTAGCACCA
CACCAGTCTGTCTCACACAACCTTTTCCGTCATTGAATCAACGATTTTCTTAAACACGTTCACATCCACACACTGAT
GGATGTAGAACAACGCATTCTGATCCTTCTTCCTCATATCACACTGAGCATTTCTTTGCGCATCCGTTGCATTTTCT
AGAAGTGAAGCATAAACTTCGTTGATGAGATCAAGAACATCTTGAGCACCAAATAACACACACATCTGAATCATCCA
ACGATTCCAGTTGTTGTCGTCGAACAATGGNAGCNTGGTGCACAGATTCACAACGATATATTATAANTTTTGTTTTA
TGAAATTTAAGAACAAATTTCCATTATTCTTAAAATGTTTACACACTGATGTAGACTGCAAAAGGAATAAAGATACA
ATTTGTTCACACCACTCACTTGCGTAAATATAAGTGAGAGTTAATGAGAAATACTAAAATACCCTCTAAAATTATGA
ATTAATTCTAACAATCTCTAATGTTAGTATAATCCATTAAACACTTTGATGGCAGGTATAACAAGGGTGTAAGTTAG
TGTATACATATTAGGCTCTTATTATTTTTATATTATCTCTGCTTTTCTTCTTCATGTTCTCACTAATATGATATTAT
CTCCCTTCCCTAAATTATTTATATTTATTAGAAAAAGAGTTTCATTTTTTAAAAATATATTACCGTAATTTTTCAAA
AAATAAAATTTAAATATATTTTATAAAAAAATTATTTAATAATTTATTTACATTAATGCATAAATATAAATAAATAC
TGTCATTTAATATTTAACCTTTTAACAATAAATTATATTTATTTAATTCAACTAATATAAGCTAAGTTATCTCATCC
AACCAATTAAAAAGATCATTTGAAAATACCTTTTTATTTAGTTTGTGGCGGTTTCAACTGTCAAAAAAAAGGAATTT
TTACGACGATATAAATTTAAACCAGCAAAAAATTGAAGCAGTTAAGCGAACCAACTCATGGTATGTGGATATATTTA
TCTTTGTCGTTTATATCGGATTCGAATCTCTATAATGATGAAAAATTAATATCAAACTTTAAATAAGAACGTCATTT
ATAGAGCCATTTTGGGAAACACATATTTCATGTACACGTGATTCGCAAATTTCCAATAACTCTATATATAGCCCTCC
TCAGTTTCATGCATTTGCTCACAACATAACCTTCCTTGAAT

A non-limiting examples of a TA29 promoter is provided as SEQ ID NO: 12:

(SEQ ID NO: 12)
CTTTTTGGTTAGCGAATGCAATTAATTTAGACATTGTGTTATGTTCCAGTTAACCGCTTCCCTGCACTTCTTTCAAT
CTATCTCTCGATAGAAAATTGTGATACTTTGCGACTTCTATCAGAGGACTTTTTGTTTTCCATGTAACAATCTGTCA
TTTTCGATGGGGAGATTTGCACAAATAGGCTATTTATGTGTCCCAATTTAAATTTTAACCCCATGTCGATCAGAACT
TAGCCACGAGCACCAGAAGTTTGATGGATATGTGACTTTGTCACTATCCGGTTTACTAATCAAGAGCTATTTTTATT
CAAAATTGGATATCTAGCTAAGTATAACTGGATAATTTGCATTAACAGATTGAATATAGTGCCAAACAAGAAGGGAC
AATTGACTTGTCACTTTATGAAAGATGATTCAAACATGATTTTTTATGTACTAATATATACATCCTACTCGAATTAA
AGCGACATAGGCTCGAAGTATGCACATTTAGCAATGTAAATTAAATCAGTTTTTGAATCAAGCTAAAAGCAGACTTG
CATAAGGTGGGTGGCTGGACTAGAATAAACATCTTCTCTAGCACAGCTTCATAATGTAATTTCCATAACTGAAATCA
GGGTGAGACAAAATTTTGGTACTTTTTCCTCACACTAAGTCCATGTTTGCAACAAATTAATACATGAAACCTTAATG
TTACCCTCAGATTAGCCTGCTACTCCCCATTTTCCTCGAAATGCTCCAACAAAAGTTAGTTTTGCAAGTTGTTGTGT
ATGTCTTGTGCTCTATATATGCCCTTGTGGTGCAAGTGTAACAGTACAACATCATCACTCAAATCAAAGTTTTTACT
TAAAGAAATTAGCTAAA

### Terminators

Aspects of the present disclosure relate to the use of a terminator within genetic constructs to terminate gene expression. The term “terminator,” as used herein, includes a section of nucleic acid sequence that marks the end of a gene or operon in DNA during transcription. A terminator sequence mediates transcriptional termination by providing signals in the newly synthesized transcript RNA that trigger processes which release the transcript RNA from the transcriptional complex. Non-limiting examples of terminators include a Noplaine Synthase (NOS) terminator and a Cauliflower Mosaic Virus (CaMV) polyadenylation (polyA) terminator.

A non-limiting example of a Noplaine Synthase (NOS) terminator is provided as SEQ ID NO: 13:

(SEQ ID NO: 13)
GATCGTTCAAACATTTGGCAATAAAGTTTCTTAAGATTGAATCCTGTTGCCGGTCTTGCGATGATTATCATATAATT
TCTGTTGAATTACGTTAAGCATGTAATAATTAACATGTAATGCATGACGTTATTTATGAGATGGGTTTTTATGATTA
GAGTCCCGCAATTATACATTTAATACGCGATAGAAAACAAAATATAGCGCGCAAACTAGGATAAATTATCGCGCGCG
GTGTCATCTATGTTACTAGATC

A non-limiting example of a CaMV PolyA terminator is provided as SEQ ID NO: 14:

(SEQ ID NO: 14)
TTTCTCCATAATAATGTGTGAGTAGTTCCCAGATAAGGGAATTAGGGTTCCTATAGGGTTTCGCTCATGTGTTGAGC
ATATAAGAAACCCTTAGTATGTATTTGTATTTGTAAAATACTTCTATCAATAAAATTTCTAATTCCTAAAACCAAAA
TCCAGTACTAAAATCCAGATC

### Flanking Sequences

Aspects of the present disclosure relate to genetic constructs comprising a left and/or right flanking sequence. The left flanking sequence may be a left border (LB) T-DNA repeat flanking the genetic information in the genetic construct. A right flanking sequence may be a right border (RB) T-DNA repeat flanking the genetic information in the genetic constructs. LB and RB T-DNA repeats are short DNA sequences on a Ti plasmid in Agrobacterium tumefaciens that mark the beginning of the transferable DNA (T-DNA) region. LB and RB T-DNA repeats are used by the Agrobacterium to recognize the T-DNA for transfer into a plant cell.

A non-limiting example of a left border (LB) T-DNA repeat is provided as SEQ ID NO: 15:

(SEQ ID NO: 15)
GTTTACACCACAATATATCCTGCCA

A non-limiting example of a right border (RB) T-DNA repeat is provided as SEQ ID NO:

(SEQ ID NO: 16)
GTTTACCCGCCAATATATCCTGTCA

### Sequence Identity

In some embodiments, a sequence of the present disclosure is a nucleotide sequence that is at least 70%, at least 75%, at least 80%, at least 85%, at least 90%, at least 95%, or at least 98% identical to any one of SEQ ID NOs: 1-16. In some embodiments, a sequence of the present disclosure is a nucleotide sequence that is 100% identical to any one of SEQ ID NOs: 1-16.

In some embodiments, a genetic construct of the present disclosure comprises a nucleotide sequence that is at least 70%, at least 75%, at least 80%, at least 85%, at least 90%, at least 95%, or at least 98% identical to any one of SEQ ID NOs: 1-4. In some embodiments, a genetic construct of the present disclosure comprises the nucleotide sequence of any one of SEQ ID NOs: 1-4.

In some embodiments, a gene of the present disclosure (e.g., heme-producing gene, barnase gene, antibiotic resistance gene, reporter gene) comprises a nucleotide sequence that is at least 70%, at least 75%, at least 80%, at least 85%, at least 90%, at least 95%, or at least 98% identical to any one of SEQ ID NOs: 5-8. In some embodiments, a gene of the present disclosure (e.g., heme-producing gene, barnase gene, antibiotic resistance gene, reporter gene) comprises the nucleotide sequence of any one of SEQ ID NOs: 5-8.

In some embodiments, a promoter of the present disclosure comprises a nucleotide sequence that is at least 70%, at least 75%, at least 80%, at least 85%, at least 90%, at least 95%, or at least 98% identical to any one of SEQ ID NOs: 9-12. In some embodiments, a promoter of the present disclosure comprises the nucleotide sequence of any one of SEQ ID NOs: 9-12.

In some embodiments, a terminator of the present disclosure comprises a nucleotide sequence that is at least 70%, at least 75%, at least 80%, at least 85%, at least 90%, at least 95%, or at least 98% identical to SEQ ID NO: 13 or SEQ ID NO: 14. In some embodiments, a terminator of the present disclosure comprises the nucleotide sequence of SEQ ID NO: 13 or SEQ ID NO: 14.

In some embodiments, a T-DNA repeat sequence of the present disclosure comprises a nucleotide sequence that is at least 70%, at least 75%, at least 80%, at least 85%, at least 90%, at least 95%, or at least 98% identical to SEQ ID NO: 15 or SEQ ID NO: 16. In some embodiments, a T-DNA repeat of the present disclosure comprises the nucleotide sequence of SEQ ID NO: 15 or SEQ ID NO: 16.

As a person having ordinary skill in the art will appreciate that any gene described herein encodes a corresponding mRNA, amino acid sequence, and polypeptide or protein.

The term “sequence identity.” as used herein, includes the relatedness of the sequences of two nucleotides when the sequences are aligned, and the term “percent identity” refers to the percentage of residues (nucleotides) that are identical when two or more nucleotide sequences are aligned. In some embodiments, sequence identity and/or percent identity is determined across the entire length of a sequence. In some embodiments, sequence identity is determined over a region (e.g., a stretch of nucleotides).

Percent identity of nucleotide sequences can be calculated by any of the methods known to one of ordinary skill in the art. For example, percent identity can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3. Where gaps exist between two sequences, Gapped BLAST® can be utilized, for example, as described in Altschul et al., Nucleic Acids Res. 25 (17): 3389-3402, 1997. When utilizing BLAST® and Gapped BLAST® programs, the default parameters of the respective programs (e.g., XBLAST®) and NBLAST®) can be used, or the parameters can be adjusted appropriately as would be understood by one of ordinary skill in the art.

A second example of a local alignment technique is based on the Smith-Waterman algorithm (Smith, T. F. & Waterman, M. S. (1981) J. Mol. Biol. 147:195-197). An example of a global alignment technique is the Needleman-Wunsch algorithm (Needleman, S. B. & Wunsch, C. D. (1970) J. Mol. Biol. 48:443-453), which is based on dynamic programming. A further example of a global alignment technique is the Fast Optimal Global Sequence Alignment Algorithm (FOGSAA).

In some embodiments, the identity of two nucleotide sequences is determined by aligning the two nucleotide sequences of the polynucleotides, calculating the number of identical nucleotides and dividing by the length of one of the nucleotide sequences.

For multiple sequence alignments, computer programs including Clustal Omega (Sievers et al., Mol Syst Biol. 2011 Oct. 11:7:539) may be used.

In preferred embodiments, a sequence, including a nucleotide sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®), NBLAST®, XBLAST® or Gapped BLAST® programs, using default parameters of the respective programs).

In some embodiments, a sequence, including a nucleotide sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) J. Mol. Biol. 147:195-197) or the Needleman-Wunsch algorithm (Needleman, S. B. & Wunsch, C. D. (1970) J. Mol. Biol. 48:443-453) using default parameters.

In some embodiments, a sequence, including a nucleotide sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleotide sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

In some embodiments, a nucleotide sequence of the present disclosure is a degenerate nucleotide sequence (e.g., a degenerate of any one of the nucleotide sequences provided herein such as degenerate of any one of SEQ ID NOs: 1-16). The term “degenerate nucleotide sequence,” as used herein, includes nucleotide sequences in which one or more positions have multiple possible bases. In some embodiments, two degenerate nucleotide sequences that are at least about 90% identical to each other encode the same amino acid sequence. In some embodiments, two degenerate nucleotide sequences that are at least about 95% identical to each other encode the same amino acid sequence. In some embodiments, two degenerate nucleotide sequences that are at least about 98% identical to each other encode the same amino acid sequence. In some embodiments, a base within a degenerate nucleotide sequence is represented by an “N.” In some embodiments, an “N” in a degenerate nucleotide sequence represents an adenine (A), a thymine (T), a guanine (G), or a cytosine (C). In some embodiments, a base within a degenerate nucleotide sequence is represented by an “R.” In some embodiments, an “R” in a degenerate nucleotide sequence represents an adenine (A) or a guanine (G). In some embodiments, a base within a degenerate nucleotide sequence is represented by a “Y.” In some embodiments, a “Y” in a degenerate nucleotide sequence represents a thymine (T) or a cytosine (C)

### Genetically Engineered Plants and Plant Cells

Aspects of the present disclosure relate to genetically engineered plants and plant cells. The term “genetically engineered,” as used herein, includes the modification of an organisms genetic makeup. Accordingly, a genetically engineered plant or plant cell is a plant or plant cell whose genetic makeup has been modified using techniques that allow for the direct transfer or removal of genes (e.g., transformation of any genetic construct(s) described herein into a plant or plant cell).

In some embodiments, a plant of the present disclosure is genetically engineered to express any genetic construct or set of genetic constructs described herein. In some embodiments, a plant is genetically engineered to express a vector comprising the nucleotide sequence of any one of SEQ ID NOs: 1-4. In some embodiments, a plant is genetically engineered to express a heme-producing gene comprising the nucleotide sequence of SEQ ID NO: 5. In some embodiments, a plant is genetically engineered to express a male-sterility gene comprising the nucleotide sequence of SEQ ID NO: 6. In some embodiments, a plant is genetically engineered to express an antibiotic resistance gene comprising the nucleotide sequence of SEQ ID NO: 5.

In some embodiments, a plant cell of the present disclosure is genetically engineered to express any genetic construct or set of genetic constructs described herein. In some embodiments, a plant cell is genetically engineered to express a vector comprising the nucleotide sequence of any one of SEQ ID NOs: 1-4. In some embodiments, a plant cell is genetically engineered to express a heme-producing gene comprising the nucleotide sequence of SEQ ID NO: 5. In some embodiments, a plant cell is genetically engineered to express a male-sterility gene comprising the nucleotide sequence of SEQ ID NO: 6. In some embodiments, a plant cell is genetically engineered to express an antibiotic resistance gene comprising the nucleotide sequence of SEQ ID NO: 5.

In some embodiments, a plant is a tomato plant. In some embodiments, a plant cell is a tomato plant cell. In some embodiments, a tomato of the present disclosure is of the genus Solanum. In some embodiments, a tomato of the present disclosure is Solanum lycopersicum. In some embodiments, a plant is an eggplant. In some embodiments, a plant cell is an eggplant cell. In some embodiments, an eggplant of the present disclosure is of the genus Solanum. In some embodiments, an eggplant of the present disclosure is Solanum melongena.

### Methods of Expressing Heme in a Plant

Aspects of the present disclosure relate to a method of transforming a plant cell with any genetic construct (e.g., vector or plasmid) described herein. Aspects of the present disclosure also relate to a method of producing a genetically engineered plant comprising introducing into a plant any genetic construct (e.g., vector or plasmid) described herein.

In some embodiments, the method of transforming or introducing is accomplished via Agrobacterium-mediated transformation. The term “transformation,” as used herein, includes the genetic alteration of a cell resulting from the direct uptake and incorporation of exogenous genetic material from its surroundings. The term “exogenous,” as used herein, includes DNA sequences originating from outside an organism that are introduced into the organism to confer new traits.

Agrobacterium-mediated transformation (AMT) is a technique used in plant biotechnology to introduce foreign DNA into plant cells and generate genetically engineered plants. AMT utilizes the natural ability of the bacterium Agrobacterium tumefaciens to transfer transfer DNA (T-DNA) into plant cells. The process involves five steps: (i) preparation of Agrobacterium, (ii) infection of plant cells, (iii) T-DNA transfer, (iv) integration into plant genome, and (v) plant regeneration. During step (i), the bacterium is transformed with a genetic construct (e.g., SEQ ID NOs: 1-4) containing the desired gene (T-DNA) and a set of genes (vir genes) that encode the machinery for T-DNA transfer. During step (ii), the transformed Agrobacterium is incubated with plant cells (e.g., tomato or eggplant), typically from seedlings, leaves, or embryos (e.g., cotyledon). In some embodiments, the transformed Agrobacterium is incubated with a cotyledon. The term “cotyledon.” as used herein, includes an embryonic leaf in seed-bearing plants, one or more of which are the first leaves to appear from a germinating seed. In some embodiments, a cotyledon is a tomato cotyledon. In some embodiments, a cotyledon is an eggplant cotyledon. In step (iii), the vir genes activate the transfer of the T-DNA from the bacterium into the plant cells. In step (iv), in some embodiments, the T-DNA integrates into the plant's genome, where it is expressed as a new gene. Finally, in step (v), the transformed plant cells are selected (e.g., using a selectable marker, e.g., a hygromycin resistance gene) and regenerated into whole plants carrying the introduced gene.

Accordingly, as described herein, aspects of the present disclosure relate to the use of Agrobacterium-mediated transformation to introduce a gene such as a heme-producing gene and/or a male-sterility gene into a plant (e.g., a tomato or eggplant).

Further aspects of the present disclosure relate to a method of producing an F1 hybrid plant (e.g., tomato or eggplant plant). The term “F1 hybrid,” as used herein, includes a first generation of offspring from two different parent plants that have been intentionally cross-pollinated to create a new, uniform variety with improved traits. In some embodiments, the method of producing an F1 hybrid plant includes crossing a genetically engineered plant of the present disclosure (e.g., a tomato plant containing SEQ ID NO: 3 or SEQ ID NO: 4) with a male-fertile plant. The term “male-fertile plant.” as used herein, includes a plant that is able to produce functional pollen and is therefore capable of self-pollination or fertilizing other plants. Unlike male-sterile plants, which are incapable of producing pollen, male-fertile plants have the necessary genes and mechanisms to complete the male reproductive process, such as the formation of functional anthers and pollen grains. In some embodiments, crossing a genetically engineered tomato plant of the present disclosure with a male-fertile plant produces a crossed plant. The term “crossed plant.” as used herein, includes a hybrid plant created by cross-pollinating two different varieties or species. In some embodiments, the method further comprises growing the crossed plant. In some embodiments, the method further comprises harvesting seeds from the crossed plant. In some embodiments, the method further comprises growing an F1 hybrid plant from the seeds harvested from the crossed plant.

In some embodiments, an F1 hybrid tomato plant of the present disclosure exhibits heme production and male sterility. In some embodiments, the F1 hybrid tomato plant exhibits increased heme production relative to a tomato plant that is not genetically engineered. Other transformation techniques used in plant biotechnology to introduce foreign DNA into plant cells and generate genetically engineered plants are contemplated herein. Non-limiting examples of transformation techniques include biolistic transformation, microinjection, electroporation, and CRISPR/Cas technology (described elsewhere herein).

The term “biolistic transformation,” as used herein, includes a method involving shooting microscopic gold or tungsten particles coated with DNA into plant cells using high-velocity gas. In some embodiments, biolistic transformation involves three steps: (i) preparation of microprojectiles, (ii) delivery mechanism, and (iii) integration of DNA. In step (i) particles, typically made of gold or tungsten, are coated with a DNA of interest. These particles serve as carriers for the genetic material. In step (ii), the coated microprojectiles are propelled into plant cells using a burst of gas, often helium. The high-velocity impact allows the particles to penetrate the rigid cell wall and membrane, delivering the DNA directly into the plant cytoplasm or nucleus. In step (iii), one inside the cell, the DNA can integrate into the plant cell genome, allowing for stable expression of the introduced genes. This integration can occur through various mechanisms, enabling the expression of new traits in the transformed plant cell.

The term “microinjection,” as used herein, includes a technique in which DNA is directly injected into the plant cell nucleus using a fine needle. In some embodiments, microinjection transformation involves four steps: (i) preparation, (ii) micromanipulation, (iii) injection, and (iv) post-injection monitoring. In step (i), the genetic material (e.g., DNA) to be injected is prepared in a solution and loaded into a micropipette, which has a fine tip to minimize damage to the plant cell during insertion. In step (ii), the plant cells are positioned under a microscope using a micromanipulator, which allows for controlled movements of the micropipette. In step (iii), the micropipette penetrates the cell membrane, and the genetic material is delivered into the cell by applying hydrostatic pressure. In step (iv), after injection, the plant cells are monitored for recovery and expression of the introduced genetic material.

The term “electroporation,” as used herein, include a method that uses an electric field to increase the permeability of a plant cell membrane, allowing DNA to enter the cell. When an electric field is applied to a plant cell, it induces temporary destabilization of the plant cell membrane, leading to the formation of hydrophilic channels or pores. The pores allow DNA to enter the cell. Once the electric field is removed, the membrane reseals, keeping the introduced DNA inside the plant cell. In some embodiments, electroporation transformation involves five steps: (i) cell preparation, (ii) DNA preparation, (iii) electroporation, (iv) recovery, and (v) culturing. In step (i), plant cells are cultured and harvested, then resuspended in an electroporation buffer to enhance conductivity and protect the cells. In step (ii), the DNA of interest is prepared in a suitable buffer. In step (iii), the plant cell-DNA mixture is placed in an electroporation cuvette, and an electric pulse is applied using an electroporator. In step (iv), after electroporation, plant cells are allowed to recover in a suitable growth medium, during which the cell membrane reseals. In step (v), the transformed plant cells are cultured to promote growth and expression of the introduced genetic material.

As a person having ordinary skill in the art will appreciate, any transformation method described herein or otherwise known in the art can be used to transform a genetic construct or set of genetic constructs described herein into a plant or plant cell (e.g., tomato plant or eggplant plant).

### Methods of Overexpressing Lycopene in a Plant

Aspects of the present disclosure relate to overexpression of lycopene in a plant (e.g., a tomato plant). Lycopene is a red carotenoid pigment found in plants, particularly tomatoes and tomato products. Lycopene has been identified as a powerful antioxidant that may provide various health benefits. Accordingly, aspects of the present disclosure relate to the overexpression of lycopene in a plant (e.g., a tomato plant) as a means to increase available lycopene in tomatoes and increase accessibility of lycopene to the public.

Lycopene is a key intermediate in the biosynthesis of many carotenoids. Within the larger biosynthetic pathway, geranylgeranyl diphosphate is converted to phytoene via the enzymatic action of phytoene synthase (Psy). Phytoene is then converted to lycopene via the enzymatic action of phytoene dehydrogenase, which is then converted to γ-carotene and then β-carotene via the enzymatic action of lycopene cyclase.

Accordingly, aspects of the present disclosure relate to method of enhancing lycopene biosynthesis in a plant (e.g. a tomato plant) by overexpressing enzymes involved in lycopene biosynthesis. In some embodiments, aspects of the present disclosure relate to overexpression of Psy. In some embodiments, a genetic construct expressing Psy is transformed into a plant (e.g., a tomato plant). In some embodiments, a genetic construct expressing Psy is transformed into a plant (e.g., a tomato plant) via Agrobacterium-mediated transformation.

Aspects of the present disclosure further relate to methods of downregulating expression of enzymes in a plant (e.g., a tomato plant) that convert lycopene to other products. In some embodiments, lycopene cyclase is downregulated. In some embodiments, gene editing system is used to downregulate expression of enzymes that convert lycopene to other products. In some embodiments, a gene editing system is used to knockdown expression of enzymes that convert lycopene to other products. In some embodiments, a gene editing system is used to knockout expression of enzymes that convert lycopene to other products.

In some embodiments, a gene editing system is a CRISPR/Cas system. In some embodiments, a gene editing system is a CRISPR/Cas9 system. In some embodiments, a gene editing system is a base editing system. In some embodiments, a gene editing system is a prime editing system.

### Gene Editing Systems

Aspects of the present disclosure relate to the use of gene editing systems to genetically engineer plants and plant cells (e.g., tomato and eggplant). In some embodiments, gene editing systems are used to integrate any genetic construct or set of genetic constructs of the present disclosure into a plant or plant cell. In some embodiments, gene editing systems are used to upregulate expression of genes that encode enzymes in the lycopene biosynthesis pathway. In some embodiments, gene editing systems are used to upregulate expression of phytoene synthase (Psy) in a plant or plant cell. In some embodiments, gene editing systems are used to downregulate (e.g., knockdown or knockout) genes that encode enzymes within pathways that compete with lycopene biosynthesis.

The term “gene editing systems,” as used herein, includes sets of gene editing molecules used for gene editing. Gene editing molecules include molecules that can precisely locate and cut nucleic acids (i.e., DNA) within a cell's genome, enabling modifications to the genome for research and agricultural applications. Non-limiting examples of gene editing molecules include programmable nucleases, guide RNAs (gRNAs), prime editing guide RNAs (pegRNAs), reverse transcriptases, DNA binding domains, DNA cleavage domains, and effector domains.

In some embodiments, plants or plant cells of the present disclosure further comprise gene editing molecules. In some embodiments, the gene editing molecules are selected from programmable nucleases. In some embodiments, the programmable nucleases are selected from Clustered Regularly Interspaced Short Palindromic Repeats (CRISPR)-Cas nucleases, Transcription Activator-Like Effector Nucleases (TALENs), Zinc Finger Nucleases (ZFNs), and meganucleases.

In some embodiments, a gene editing molecule is a programmable nuclease. A “programmable nuclease” includes engineered enzymes that can be used to cut DNA sequences. Non-limiting examples of programmable nucleases include Cas nucleases, TALENs, ZFNs, and meganucleases.

In some embodiments, a gene editing molecule is a guide RNA (gRNA). A “guide RNA (gRNA)” is a short RNA sequence that directs a Cas nuclease to a specific DNA location for genome editing. In some embodiments, a gene editing molecule is a prime editing guide RNA (pegRNA). A “prime editing guide RNA (pegRNA)” is a specific type of gRNA that directs a Cas nickase to a specific DNA location for genome editing. In some embodiments, a gene editing molecule is a reverse transcriptase. A “reverse transcriptase (RT)” is an enzyme that catalyzes the process of reverse transcription, converting RNA into DNA. In some embodiments, a gene editing molecule comprises a DNA binding domain. A “DNA binding domain” is a protein region that specifically recognizes and binds to DNA, often acting as a part of a transcription factor or other DNA-interacting protein. In some embodiments, a gene editing molecule comprises a DNA cleavage domain. A “DNA cleavage domain” is a specific region within a protein (e.g., FokI restriction enzyme or a Cas nuclease) that is responsible for cutting or cleaving DNA strands. DNA cleavage domains, often part of larger multi-domain proteins, mediate the enzymatic activity of cutting DNA. In some embodiments, a gene editing molecule comprises an effector domain. An “effector domain” is a protein region that mediates the activity of a transcription factor by interacting with other proteins and/or DNA to regulate gene expression, either activating or repressing transcription.

Gene editing molecules are typically part of a larger gene editing system in which two or more components (i.e., gene editing molecules) function in tandem to edit a cell's genome. Non-limiting examples of gene editing examples include a CRISPR-Cas system, a base editing system, a prime editing system, a Transcription Activator-Like Effector Nuclease (TALEN) system, a Zinc Finger Nuclease (ZFN) system, and a Meganuclease system.

### CRISPR-Cas Systems

In some embodiments, gene editing molecules comprise a CRISPR-Cas nuclease and a guide RNA (gRNA). In some embodiments the CRISPR-Cas nuclease is a Cas9 nuclease. In some embodiments, the CRISPR-Cas nuclease is a Cas12 nuclease. In some embodiments, the CRISPR-Cas nuclease is a dead Cas9 (dCas9) nuclease.

Aspects of the present disclosure relate to the use of a CRISPR-Cas (Clustered Regularly Interspaced Short Palindromic Repeats-CRISPR-associated nuclease) system as a gene editing system. A CRISPR-Cas system is a gene editing system derived from a bacterial immune system, which allows for precise editing of DNA by cutting and/or modifying sequences. In some embodiments, a CRISPR-Cas system comprises a Cas nuclease (e.g., Cas9 nuclease), a CRISPR RNA (crRNA), and a trans-activating CRISPR RNA (tracrRNA). In other embodiments, a CRISPR-Cas system comprises a Cas nuclease (e.g., Cas9 nuclease) and an sgRNA, which is an engineered fusion of a crRNA and a tracrRNA. A crRNA includes an RNA sequence complementary to a target DNA and guides the Cas nuclease to the correct genomic target location. A tracrRNA binds to crRNA to stabilize and guide a Cas-crRNA complex. An sgRNA simplifies a CRISPR-Cas system by combining the crRNA and tracrRNA components. The term “gRNA” encompasses any one or more RNA that includes an RNA sequence complementary to a target DNA and is capable of guiding a Cas nuclease to a genomic target location, such as a sgRNA or the combination of a crRNA and a tracrRNA.

A CRISPR-Cas system, in some embodiments, includes a gRNA that recruits a Cas nuclease to a specific site in a cell's genome to generate a double-stranded break (DSB), which can be repaired by two endogenous self-repair mechanisms, the error-prone non-homologous end joining (NHEJ) pathway or the homology-directed repair (HDR) pathway. NHEJ can introduce random insertions or deletions (indels) into the cleavage sites, leading to the generation of frameshift mutations or premature stop codons within the open reading frame (ORF) of the target genes, finally inactivating the target gene. Alternatively, HDR can introduce precise genomic modifications at the target site by using a homologous DNA repair template. CRISPR-Cas systems are well-known in the art.

In some embodiments, a CRISPR-Cas system comprises a Cas nuclease. Cas nucleases are enzymes that cut DNA at specific locations. Non-limiting examples of Cas nucleases include Cas9 nucleases, Cas12 nucleases, and CasX and CasY nucleases. In some embodiments, a Cas nuclease is a Cas9 nuclease. Cas9 nucleases include enzymes originally derived from Streptococcus pyogenes. In some embodiments, a Cas nuclease is a Cas12 nuclease, for example, a Cas12a, Cas12b, Cas12c, Cas12d, Cas12e, Cas12f, or Cas12g nuclease. Cas12a nucleases include enzymes originally derived from Francisella novicida. A CRISPR-Cas system that includes a Cas12a nuclease, in some embodiments, involves a crRNA for delivery of the Cas12a nuclease to a target sequence. In some embodiments, a Cas nuclease is a dead Cas nuclease (dCas) (e.g., a dead Cas9 (dCas9) nuclease). A “dCas” nuclease is a variant of a Cas nuclease that lacks the DNA-cleaving activity of the Cas nuclease, allowing for targeted gene regulation without causing DNA mutations. In some embodiments, a dCas is a dCas9. Other “dead” Cas nucleases may be used in accordance with the methods and compositions described herein.

### Base Editing Systems

Aspects of the present disclosure relate to the use of a base editing system as a gene editing system. A base editing system includes a gene editing system that was developed to improve the efficiency of site-directed mutagenesis. Base editing systems contain a modified Cas nuclease (e.g., a dCas9) coupled with a deaminase, such as a cytosine deaminase (cytidine base editor; CBE) or adenosine deaminase (adenine base editor; ABE) and can introduce C·G to T·A or A·T to G·C point mutations into an editing window of a gRNA target site without double-stranded DNA cleavage. The term “editing window” includes the specific region on the DNA where Cas-based base editors modify DNA bases. Base editing systems are well-known in the art. For example, see Rees H A, Liu D R. Nat Rev Genet. 2018 December; 19 (12): 770-788; Erratum in: Nat Rev Genet. 2018 December; 19 (12): 801.

Aspects of the present disclosure relate to the use of a base editing system. A base editing system can include, for example, gene editing molecules that enable precise, single-nucleotide changes in DNA without making double-strand breaks or requiring a donor DNA template. In some embodiments, a base editing system comprises a binding protein, a gRNA (described elsewhere herein), and a deaminase. “Binding protein” includes proteins that selectively binds to another molecule, such as DNA. In some embodiments, a binding protein is a modified Cas protein, for example, a dCas nuclease or a Cas nickase (nCas). In some embodiments, a binding protein is dCas9 nuclease. “Deaminases” include enzymes that remove an amino group from a molecule, typically a nucleotide base. Non-limiting examples of deaminases include cytidine deaminases and adenosine deaminases. In some embodiments, a deaminase includes a cytosine deaminase. In other embodiments, a deaminase includes an adenosine deaminase.

In some embodiments, a cytosine deaminase comprises a cytidine base editor. A cytidine base editor is capable of modifying a single base in DNA without cutting the DNA. In some embodiments, a cytosine deaminase introduces C·G to T·A point mutations into an editing window of a gRNA target site without double-stranded DNA cleavage. In some embodiments a deaminase is an adenosine deaminase. In some embodiments, an adenosine deaminase comprises an adenine base editor. In some embodiments, an adenosine deaminase induces A T to G·C point mutations into an editing window of a gRNA target site without double-stranded DNA cleavage.

### Prime Editing Systems

Aspects of the present disclosure relate to the use of a prime editing system as a gene editing system. A prime editing system includes a gene editing system that can mediate targeted insertions, deletions, and all 12 types of base substitutions without double-strand breaks or donor DNA templates. A prime editing system contains a modified Cas nuclease (e.g., a Cas nickase (nCas) that cuts only one DNA strand) fused to a reverse transcriptase and a prime editing gRNA (pegRNA) with functions of specifying a target site and encoding a desired edit. Typically, after a Cas nuclease (e.g., Cas9) cleaves a target site, the reverse transcriptase uses pegRNA as a template for reverse transcription and new genetic information can be written into the target site. Prime editing systems are well-known in the art. For example, see Anzalone, A. V., et al. Nature 576, 149-157 (2019).

In some embodiments, a prime editing system comprises a binding protein, a pegRNA, and a reverse transcriptase. Unlike a standard gRNA, a pegRNA also carries a primer binding site (PBS) and a reverse transcriptase template (RTT), which are necessary for prime editing to occur. A “reverse transcriptase” is an enzyme that copies RNA into DNA, a process called reverse transcription.

In some embodiments, a binding protein is a Cas9 nickase (nCas9). An “nCas9” is a modified version of the Cas9 enzyme that, instead of cutting both strands of DNA, creates a single strand break (a “nick”) at the target site. Use of an nCas9 reduces off-target effects and enhances genome editing precision.

### TALEN Systems

Aspects of the present disclosure relate to the use of a TALEN (Transcription Activator-Like Effector Nuclease) system as a gene editing system. A TALEN system is a gene editing system that utilizes a TALEN, a type of engineered nuclease derived from bacterial TALE proteins, for precise gene editing. TALE proteins can be designed to target specific DNA sequences for modification. TALENs consist of two parts: a DNA-binding domain derived from bacterial TALE proteins and a DNA-cleaving domain. In some embodiments, a DNA-cleaving domain is a FokI nuclease. The TALE protein domain allows TALENs to bind to specific DNA sequences, while the FokI nuclease creates a double-strand break at that location. TALEN systems are well-known in the art. For example, see Joung J K, Sander J D. TALENs: a widely applicable technology for targeted genome editing. Nat Rev Mol Cell Biol. 2013 January; 14 (1): 49-55, the contents of which are herein incorporated by reference in their entirety.

In some embodiments, a TALEN system comprises a designed TALE protein fused to a DNA-cleaving domain. In some embodiments, a DNA-cleaving domain is the cleavage domain of the FokI nuclease.

### Zinc Finger Nuclease Systems

Aspects of the present disclosure relate to the use of a zinc finger nuclease system as a gene editing system. A zing finger nuclease system is a gene editing system that utilizes Zinc Finger Nuclease (ZFN) technology. A ZFN is an artificial endonuclease that consists of a designed zinc finger protein (ZFP) fused to the cleavage domain of the FokI restriction enzyme. A ZFN can be redesigned to cleave new targets by developing ZFPs with new sequence specificities. ZFN systems are well-known in the art. For example, see Urnov, F., Rebar, E., Holmes, M. et al. Genome editing with engineered zinc finger nucleases. Nat Rev Genet 11, 636-646 (2010), the contents of which are herein incorporated by reference in their entirety.

In some embodiments, a ZFN system comprises a ZFP fused to a DNA-cleaving domain. In some embodiments, a DNA-cleaving domain is the cleavage domain of the FokI nuclease.

### Meganuclease Systems

Aspects of the present disclosure relate to the use of a meganuclease system as a gene editing system. A meganuclease system is a gene editing system that utilizes meganucleases. Meganucleases, also known as homing endonucleases, are sequence-specific endonucleases that recognize and cleave long DNA sequences (e.g. about 12 to about 40 base pairs in length). Meganucleases induce double-strand breaks, facilitating homologous recombination for gene targeting and modification. Meganuclease systems are well-known in the art. For example, see Silva G, Poirot L, Galetto R. Smith J, Montoya G, Duchateau P. Pâques F. Meganucleases and other tools for targeted genome engineering: perspectives and challenges for gene therapy. Curr Gene Ther. 2011 February; 11 (1): 11-27, the contents of which are herein incorporated by reference in their entirety. In some embodiments, a meganuclease system comprises a meganuclease.

### Kits

The genetic constructs described herein may, in some embodiments, be assembled into kits to facilitate their use in research and agricultural applications. A kit may include one or more containers housing the components (e.g., a genetic construct or set of genetic constructs) of the disclosure and instructions for use. Specifically, such kits may include one or more agents described herein, along with instructions describing the intended application and the proper use of these agents. Kits for research purposes may contain the components in appropriate concentrations or quantities for performing various experiments.

The kit may be designed to facilitate use of the methods described herein by researchers and can take many different forms. Each of the compositions of the kit, where applicable, may be provided in liquid form (e.g., in solution), or in solid form (e.g., a dry powder). In certain cases, some of the compositions may be constitutable or otherwise processable (e.g., to an active form), for example, by the addition of a suitable solvent or other medium (for example, water or a cell culture medium), which may or may not be provided in the kit. As used herein, “instructions” can include a component of instruction and/or promotion, and typically involve written instructions on or associated with the packaging. Instructions also can include any oral or electronic instructions provided in any manner such that a user will clearly recognize that the instructions are to be associated with the kit, for example, audiovisual (e.g., videotape, DVD, CD-ROM, website links for downloadable file), internet, and/or web-based communications, etc. The written instructions may be in a form prescribed by a governmental agency regulating the manufacture, use, or sale of biological products, which instructions can also reflect approval by the agency of manufacture, use, or sale for plant administration.

The kit may contain any one or more of the components described herein in one or more containers. As an example, in one embodiment, the kit may include instructions for mixing one or more components of the kit and/or isolating and mixing a sample and applying it to a plant. The kit may include a container housing the compounds described herein. The compounds may be in the form of a liquid, gel, or solid (powder). The compounds may be prepared sterilely, packaged in a syringe, and shipped refrigerated. Alternatively, the compounds may be housed in a vial or other container for storage. A second container may have other agents prepared sterilely. Alternatively, the kit may include the compounds premixed and shipped in a syringe, vial, tube, or other container.

### EXAMPLES

In order that the disclosure described in the present application may be more fully understood, the following examples are set forth. The examples described in this application are offered to illustrate the systems and methods provided in this disclosure and are not to be construed in any way as limiting their scope.

### Example 1. Genetic Construct Design

This Example relates to a dual-gene construct designed to simultaneously express a heme-producing protein and a male-sterility gene within a single transformation event. A non-limiting example of the dual-gene construct described in this Example is provided by FIG. 3. Genetic constructions used throughout this Examples section are shown in FIGS. 4-7.

The genetic construct described in this Example comprises a heme gene expression cassette, a male sterility expression cassette, and a selectable marker expression cassette.

### Heme Gene Expression Cassette

The Heme Gene Expression Cassette described herein includes a promoter, a gene, and a terminator. The promoter is a Cauliflower Mosaic Virus 35S (CaMV 35S) promoter, a strong promoter used to drive constitutive expression of the heme gene in a tomato or eggplant. The gene is the leghemoglobin gene, derived from Glycine max (soybean), or functional homolog thereof, for efficient heme production, to introduce desirable meat-like flavor and enhance iron content. The terminator is a Noplaine Synthase (NOS) terminator to ensure proper transcription termination and mRNA stability of the heme gene.

### Male Sterility Expression Cassette

The Male Sterility Expression Cassette described herein includes a promoter, a gene, and a terminator. The promoter is an anther-specific promoter, such as TA29 promoter from tobacco, to drive expression of male sterility gene within the anther tissue of the flower. The gene is the barnase gene from Bacillus amyloliquefaciens to introduce male sterility by disrupting pollen development. The terminator is a NOS terminator to ensure proper transcription termination and mRNA stability of the barnase gene.

### Selectable Marker Cassette

The Selectable Marker Cassette described herein includes the hygromycin phosphotransferase (hpt) gene to confer hygromycin resistance is included within the T-DNA region of the expression cassette to enable selection of successfully transformed plant cells. This gene is regulated by CaMV 35S promoter and utilizes a CaMV 35S polyadenylation (polyA) signal to ensure proper transcript termination and stability. This selectable marker allows for efficient identification and regeneration of transgenic plants on media containing hygromycin.

### Example 2. Transformation and Regeneration of Tomato Plants

Transformation of the desired tomato varieties, (including Rutgers, Brandywine, and Marglobe), and/or eggplant varieties (including Black Beauty, Rosa Bianca, and Nadia), may be achieved through Agrobacterium-mediated gene transfer. Sterile explants such as cotyledons from young seedlings are co-cultivated with Agrobacterium tumefaciens containing the dual-gene construct described in the present disclosure under optimized culture conditions. Following co-cultivation, explants are transferred to selection media containing (i) cefotaxime to eliminate Agrobacterium and (ii) hygromycin to allow regeneration of successfully transformed plant tissue. Callus formation is induced used cytokinin-enriched medium, followed by shoot regeneration and root induction. Successfully regenerated transgenic plants are acclimatized under controlled environments before being transferred to the necessary soil or hydroponic systems.

### Methods

### Seed Germination (0-10 Days);

Tomato seeds were prepared for germination by sterilizing in 20% (v/v) bleach, <0.01% Tween-20, and agitating for 20 min at 250 rpm. Seeds were then rinsed 5 times in distilled water (dH₂O), lightly dried, and sown into Murashige and Skoog (MS) medium (2.15 g/L MS Salts, 0.1 g/L myoinositol, 29.2 mM sucrose, 5.93 uM Thiamine-HCl, 4.86 uM Pyridoxine-HCl, 8.12 uM Nicotinic Acid, 8 g/L Agar, pH=5.8). Seeds were incubated for 48-72 hr in dark, and then transferred to photoperiod incubation at 25° C., 16 hr light/8 hr dark.

### Cotyledon Explant Preparation (1-3 Days);

Cotyledons that formed 8-day-old seedlings were excised at the petiole, using a sterile blade to cut ˜5-10 mm explants. Cotyledons were placed adaxial side down on Preculture Medium, (4.3 g/L MS Salts, 0.1 g/L myoinositol, 1 mL/L Nitsch Vitamins, 20 g/L Sucrose, 5.2 g/L Gelzan-Agar, pH=5.8, 2 mg/L Trans-Zeatin), and incubated for 48 hr in the dark at 19° C.

### Agrobacterium-Mediated Transformation

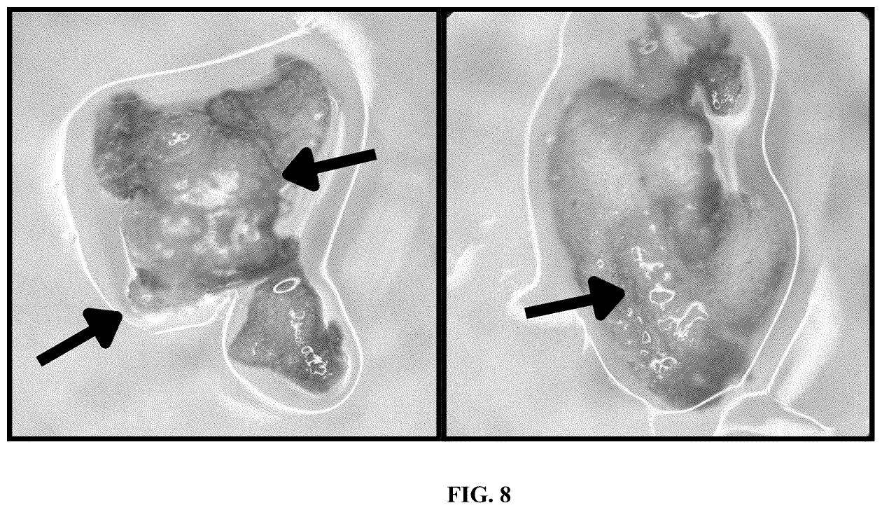

Agrobacterium colonies were inoculated in 50 mL of YEP medium with antibiotic selection according to the plasmid vector used. Cultures were grown to an OD600=0.6-0.8 in 50 mL media, and then pelleted and resuspended into liquid Preculture Medium. Prepared cotyledon explants were incubated with Agrobacterium co-culture for ˜20 min with gentle agitation. Explants were then blotted on sterile filter paper and returned to Preculture Medium agar plates, adaxial side down, and incubated for 72 hr in dark conditions at 19° C. Cotyledons were then transferred to fresh Regeneration Media agar plates (Preculture Medium, supplemented with Hygromycin), adaxial side up, and incubated in photoperiod incubator for 10 days. Plates were refreshed every 10-14 days until callus formation and shoot induction. FIG. 8 shows microscope images depicting optimized conditions for tomato regeneration in control cotyledons with callus formation in 4 week cotyledon explants. Arrows point at early callus, characterized by translucent tissue that accumulates on the adaxial side of leaf.

### Example 3. Molecular and Phenotype Validation

Transgenic plants are verified at both molecular and phenotypic levels. Genomic DNA is extracted and verified for successful incorporation of both the heme gene and male sterility gene by PCR and amplicon sequencing. Phenotypic outcome is validated by standard chromatography and spectrophotometry techniques to examine increased levels of heme and iron in transgenic plants versus wild-type plants. Male sterility is confirmed by microscopy of anther morphology and pollen viability assays.

### Example 4. F1 Hybrid Seed Production

The male-sterile, heme-producing plants (Parental Line A) are used as the female parent in controlled cross-breeding with selected male-fertile plants (Parental Line B). Due to male sterility, Parental Line A cannot self-pollinate, ensuring that all resulting seeds are true F1 hybrids. Seeds are harvested from the crossed plant and used to produce F1 hybrid offspring, and then further characterized by uniform growth, hybrid vigor, and enhanced flavor.

### Example 5. Lycopene Enhancement Strategies

This Example relates to approaches for improving lycopene biosynthesis in tomato fruits through genetic or conventional means.

### Method 1

Genetic modification can enhance lycopene production by upregulating the expression of major proteins involved in the biosynthesis pathway such as phytoene synthase (Psy), and/or coupled with modification of genes such as phytoene desaturase (Pds) and zeta-catorene desaturase (Zds) to enhance lycopene production. These genes are regulated by the E8 promoter to ensure lycopene overproduction is targeted to the fruit ripening stage. Additionally, competing carotenoid pathway genes may be downregulated to increase metabolic flux toward lycopene accumulation.

### Method 2

Crossbreeding high-lycopene public-domain varieties, such as Tangerine or Health Kick tomatoes, with the genetically modified line yields hybrids combining natural lycopene enhancement with high heme content and hybrid vigor.

### Example 6. Agronomic Evaluation and Commercial Application

Transgenic and hybrid plants are evaluated under greenhouse and field trials to assess yield, characterize fruit production and quality, and profile the flavor and nutrient content. Data collection and analysis includes wild-type controls to confirm elevated lycopene and iron content.

The genetically engineered male-sterile line simplifies commercial hybrid seed production by eliminating manual emasculation, reducing labor costs, and improving uniformity. Furthermore, the resulting fruit offers a unique flavor profile with nutritional and environmental advantages aligned with the global shift toward plant-based, functional foods.

### Additional Embodiments

1. A genetically modified vegetable plant or plant cell comprising:
   a heme gene expression cassette, including a promoter, a heme-producing gene, and a first terminator.
2. The plant or cell of Embodiment 1, wherein the vegetable is a tomato or an eggplant.
3. The plant or cell of Embodiment 1 or 2 further including a male sterility gene expression cassette, including an anther-specific promoter, a male sterility gene, and a second terminator.
4. A method of producing a genetically modified vegetable plant or plant cell comprising a step of:
   introducing a genetic construct comprising a heme gene and/or a male sterility gene into the plant or cell to produce a transformed plant or cell.
5. The method of Embodiment 4, wherein the introducing occurs via Agrobacterium tumefaciens.
6. The method of Embodiment 4 or 5 further comprising selecting and regenerating the transformed plant.
7. The method of any one of Embodiments 4-6 further comprising verifying an expression of both the heme gene and/or the male sterility gene.
8. The method of any one of Embodiments 4-7, wherein the vegetable includes tomato or an eggplant.
9. A method of use of a genetically modified vegetable plant of any one of Embodiments 1-3 as a female parent in the production of F1 hybrid seeds.
10. The method of Embodiment 9, wherein the vegetable includes a tomato or an eggplant.
11. A genetically modified plant (e.g., tomato) wherein a heme gene contained therein includes a leghemoglobin gene from Glycine max and a male sterility gene contained therein includes a barnase gene from Bacillus amyloliquefaciens.
12. The genetically modified plant of Embodiment 11, wherein an anther-specific promoter includes a TA29 promoter from tobacco.
13. A method for producing F1 hybrid seeds comprising steps of:
   (i) crossing a genetically modified plant (e.g., tomato) as claimed in claim 2 with a male-fertile plant to produce a crossed plant;
   (ii) harvesting seeds from the crossed plant; and
   (iii) planting the seeds to grow F1 hybrid plants.
14. An F1 hybrid plant (e.g., tomato) produced by the method of Embodiment 13, wherein the plant exhibits both heme production and male sterility.
15. A method for enhancing lycopene content in plant fruits (e.g., tomatoes), comprising: introducing a genetic construct that overexpresses the phytoene synthase (Psy) gene under the control of a fruit-specific promoter, thereby increasing lycopene biosynthesis during fruit ripening.
16. A method for increasing lycopene production in plants (e.g., tomatoes), comprising: using CRISPR/Cas9 to knock out genes that compete with the lycopene biosynthesis pathway, ensuring that metabolic precursors are directed towards lycopene production.
17. A method for producing hybrid plants (e.g., tomatoes) with enhanced lycopene content, comprising: crossbreeding public domain high-lycopene varieties to produce hybrids with increased lycopene content and desirable fruit qualities such as large size, flavor, and/or high yield.
18. The method of Embodiment 15, wherein the fruit-specific promoter is the E8 promoter, ensuring lycopene overproduction specifically during the ripening stage.
19. The method of Embodiment 17, wherein the public domain varieties (e.g., tomatoes) are selected from Tangerine, Double Rich, or Health Kick, known for their naturally elevated lycopene content.

### EQUIVALENTS

Those skilled in the art will recognize or be able to ascertain using no more than routine experimentation, many equivalents to the specific embodiments of the invention described in the present application. Such equivalents are intended to be encompassed by the following claims. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Such equivalents are intended to be encompassed by the following claims. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise. All references, including patent documents, are incorporated by reference in their entirety.

What is claimed is:

1. A genetic construct or set of genetic constructs comprising a first promoter operably linked to a first coding sequence and a second promoter operably linked to a second coding sequence, wherein the first coding sequence encodes a heme-producing gene, and wherein the second coding sequence encodes a male-sterility gene.

2. The genetic construct or set of genetic constructs of claim 1, wherein the genetic construct comprises the nucleotide sequence of SEQ ID NO: 3 or SEQ ID NO: 4.

3. The genetic construct or set of genetic constructs of claim 1, wherein the heme-producing gene is a leghemoglobin gene derived from Glycine max.

4. The genetic construct or set of genetic constructs of claim 3, wherein the leghemoglobin gene comprises the nucleotide sequence of SEQ ID NO: 5.

5. The genetic construct or set of genetic constructs of claim 1, wherein the male-sterility gene is a barnase gene derived from Bacillus amyloliquefaciens.

6. The genetic construct or set of genetic constructs of claim 5, wherein the barnase gene comprises the nucleotide sequence of SEQ ID NO: 6.

7. The genetic construct or set of genetic constructs of claim 1, wherein the first promoter and/or the second promoter is a constitutive promoter.

8. The genetic construct or set of genetic constructs of claim 7, wherein the constitutive promoter comprises the nucleotide sequence of SEQ ID NO: 9.

9. The genetic construct or set of genetic constructs of claim 1, wherein the first promoter and/or the second promoter is a plant promoter.

10. The genetic construct or set of genetic constructs of claim 9, wherein the plant promoter comprises the nucleotide sequence of any one of SEQ ID NOs: 10-12.

11. The genetic construct or set of genetic constructs of claim 1 further comprising a terminator sequence, wherein the terminator sequence comprises the nucleotide sequence of SEQ ID NO: 13 or SEQ ID NO: 14.

12. The genetic construct or set of genetic constructs of claim 1 further comprising an antibiotic resistance gene.

13. The genetic construct or set of genetic constructs of claim 12, wherein the antibiotic resistance gene is a hygromycin resistance gene.

14. The genetic construct or set of genetic constructs of claim 13, wherein the hygromycin resistance gene comprises the nucleotide sequence of SEQ ID NO: 7.

15. The genetic construct or set of genetic constructs of claim 1 further comprising a first flanking sequence and a second flanking sequence, wherein the first flanking sequence comprises the nucleotide sequence of SEQ ID NO: 15 and wherein the second flanking sequence comprises the nucleotide sequence of SEQ ID NO: 16.

16. A plant cell, comprising the genetic construct or set of genetic constructs of claim 1.

17. The plant cell of claim 16, wherein the plant cell is tomato plant cell or an eggplant plant cell.

18. A method, the method comprising transforming a plant cell with the genetic construct or set of genetic constructs of claim 1.

19. The method of claim 18, wherein transforming is via Agrobacterium-mediated transformation.

20. The method of claim 18, wherein the plant cell is tomato plant cell or an eggplant plant cell.

21. A method of producing a genetically engineered plant, the method comprising introducing into a plant the genetic construct or set of genetic constructs of claim 1.

22. The method of claim 21, wherein the introducing is via Agrobacterium-mediated transformation.

23. The method of claim 21, wherein the genetically engineered plant is a tomato plant or an eggplant plant.

24. A method of producing an F1 hybrid tomato plant, the method comprising:
(i) crossing the genetically engineered plant of claim 21 with a male-fertile tomato plant to produce a crossed plant;
(ii) harvesting seeds from the crossed plant; and
(iii) growing the F1 hybrid tomato plant from the seeds.
